(19) United States
(12) Patent Application Publication
(10) Pub. No.: US 2025/0262315 A1
(43) Pub. Date: Aug. 21, 2025

(54) TRIPEPTIDE LINKERS AND METHODS OF USE THEREOF

(71) Applicant: THE BOARD OF REGENTS OF THE UNIVERSITY OF TEXAS SYSTEM, Austin, TX (US)

(72) Inventors: Kyoji TSUCHIKAMA, Manvel, TX (US); Yasuaki ANAMI, Manvel, TX (US); Yin Yuen HA, Houston, TX (US); Chisato TSUCHIKAMA, Manvel, TX (US); Zhiqiang AN, Houston, TX (US); Ningyan ZHANG, Houston, TX (US)

(21) Appl. No.: 18/722,251

(22) PCT Filed: Dec. 20, 2022
(86) PCT No.: PCT/US2022/082020

§ 371 Date: Jun. 20, 2024

Related U.S. Application Data
(60) Provisional application No. 63/291,918, filed on Dec. 20, 2021.

(51) Int. Cl. A61K 47/68; A61P 35/00

(52) U.S. Cl. CPC A61K 47/68031; A61K 47/6855; A61K 47/6889; A61P 35/00

(57) ABSTRACT

Provided herein are peptide linkers which may be used to prepare drug conjugates, drug conjugates prepared using these linkers, and compositions and methods of treatment thereof.

This application claims the benefit of priority to U.S. Provisional Application No. 63/291,918, filed on Dec. 20, 2021, the entire contents of which are hereby incorporated by reference.

### BACKGROUND OF THE INVENTION

### I. Field of the Invention

This disclosure relates to the fields of medicine, pharmacology, chemistry, and oncology. In particular, compounds, compositions, methods of diagnosis, methods of treatment, and methods of synthesis relating to antibody-drug conjugates (ADCs) are disclosed.

### II. Description of Related Art

Targeted drug delivery has attracted increasing attention as a means to improve drug efficacy while reducing toxicity to healthy tissues. In particular, antibody-drug conjugates (ADCs), monoclonal antibodies (mAbs) linked with pharmacologically active molecules (payloads) via chemical linkers, are one of the most promising classes for the treatment of cancers and other diseases with remarkable and durable treatment effects. The clinical success of this drug class has been demonstrated with eleven U.S. Food and Drug Administration (FDA)-approved ADCs for a broad range of hematological malignancies and solid tumors and more than 100 candidates in clinical trials. Despite recent advances in ADC chemistry (e.g., novel linkers, payloads, conjugation methods), cancer biology, and clinical management, ADC-based treatment is often associated with various side effects, including myelosuppression and liver toxicity. Thus, ADC technologies capable of minimizing the risk of adverse effects are strongly desired to implement effective cancer therapy without impairing patients' quality of life.

The ADC linker is an important component influencing the overall efficacy and safety profiles. Cleavable linkers are used for nearly 80% of ADCs to efficiently liberate conjugated payloads inside the target cancer cells, leading to increased ADC potency. Among them, cathepsin-sensitive valine-citrulline (VCit) and similar dipeptides linkers connecting a payload with or without a p-aminobenzyloxycarbonyl (PABC) are most commonly used as an industry-standard technology for more than 40 ADCs, including ADCETRIS®, POLIVY®, PADCEV®, and ZYNLONTA® (FIG. 1A). However, their susceptibility to extracellular carboxylesterase 1c (Ces1c) in rodent plasma causes premature payload release in circulation, complicating preclinical evaluation using rodent models. More importantly, instability of this valine-based linker is likely associated with high frequency of dose-limiting antigen-independent toxicities in clinical applications. In Phases II and III studies for ADCETRIS®, an ADC equipped with monomethyl auristatin E (MMAE) through VCit linkers, neutropenia (16-22% of patients) or hepatic toxicity (7% of patients) were common side effects leading to dose delay or treatment discontinuation. Thus, there is an unmet need to develop improved chemical linkers, such as for ADCs.

### SUMMARY

In some aspects, the present disclosure provides peptide linkers which may be used to prepare drug conjugates, drug conjugates prepared using these linkers, and compositions and methods of treatment thereof.

In some aspects, the present disclosure provides compounds of the formula:

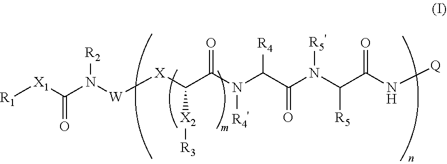

wherein:
   X₁ is a covalent bond, alkanediyl_{(C≤12)}, or substituted alkanediyl_{(C≤12)};
   R₁ is hydrogen, —ZR₆, —(OCH₂CH₂)ₙZR₆, or substituted —(OCH₂CH₂)ₙZR₆, wherein:
      n is 0-50; and
      R₆ is hydrogen, hydroxy, aminohydroxy, amino, mercapto, hydroxylamino, hydrazino, or azide; or
         alkyl_{(C≤12)}, alkenyl_{(C≤12)}, alkynyl_{(C≤12)}, alkylhydrazine_{(C≤12)}, or a substituted version of any of these groups;
         a polyglycine comprising from 1 to 6 glycine units; or
         a substructure of the formula:

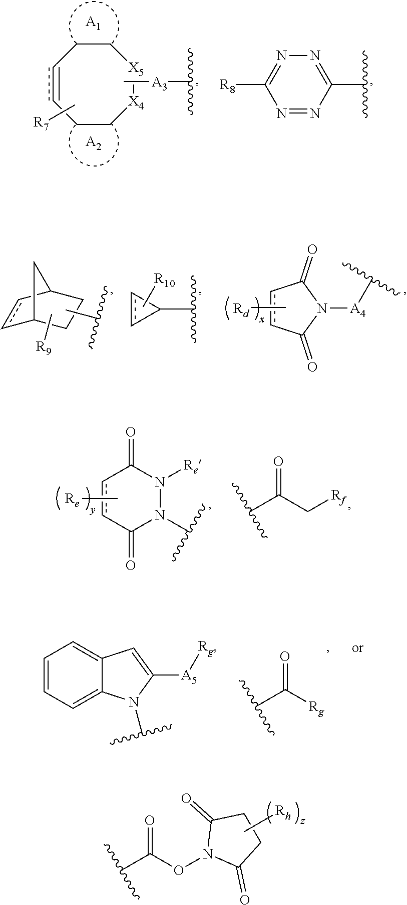

wherein:
A₁ and A₂ are each independently absent or arenediyl_{(C≤12)}, substituted arenediyl_{(C≤12)}, heteroarenediyl_{(C≤12)}, or substituted heteroarenediyl_{(C≤12)}, and form a fused arene_{(C≤12)}, substituted arene_{(C≤12)}, heteroarene_{(C≤12)}, or substituted heteroarene_{(C≤12)};
A₃ is a covalent bond, O, alkanediyl_{(C≤8)}, substituted alkanediyl_{(C≤8)}, alkoxydiyl_{(C≤8)}, substituted alkoxydiyl_{(C≤8)},
A₄ or A₅ are each independently selected from a covalent bond, alkanediyl_{(C≤8)}, substituted alkanediyl_{(C≤8)}, arenediyl_{(C≤8)}, or substituted arenediyl_{(C≤8)};
R_{d}, Rₑ, Rₑ′, and Rₕ are each independently selected from hydrogen, halo, thioether, selenoether, sulfate, tosylate, mesylate, aryl_{(C≤8)}, or substituted aryl_{(C≤8)};
R_{f} is halo;
R_{g} is amine, hydrazine, alkylamino_{(C≤8)}, substituted alkylamino_{(C≤8)}, dialkylamino_{(C≤8)}, substituted dialkylamino_{(C≤8)}, alkylhydrazine_{(C≤8)}, or substituted alkylhydrazine_{(C≤8)};
X₄ and X₅ are each independently O, N, C(O), CH₂, or X₄ and X₅ are alkanediyl_{(C≤8)} or substituted alkanediyl_{(C≤8)} and are taken together to form a fused cycloalkane group consisting of 3 to 8 ring atoms;
R₇ is hydrogen, hydroxy, amino, or oxo;
R₈ is carboxy; or
alkyl_{(C≤12)}, amido_{(C≤12)}, aryl_{(C≤12)}, heteroaryl_{(C≤12)}, —C(O)OR₁₁, —C(O)NR₁₁R₁₁′, or a substituted version of any of these groups wherein:
R₁₁ and R₁₁′ are each independently hydrogen; or
alkyl_{(C≤12)}, aryl_{(C≤12)}, or a substituted version of either of these groups;
R₉ and R₁₀ are each independently hydroxy, amino, halo; or
alkyl_{(C≤12)}, aryl_{(C≤12)}, or a substituted version of either of these groups;
Z is a covalent bond, alkanediyl_{(C≤12)}, —C(O)-alkanediyl_{(C≤12)}, —C(O)-alkanediyl_{(C≤12)}-C(O)NH—, or a substituted version of any of these groups;
R₂ is hydrogen, alkyl_{(C≤12)}, substituted alkyl_{(C≤12)}, aryl_{(C≤12)}, heteroaryl_{(C≤12)}, aralkyl_{(C≤12)}, heteroaralkyl_{(C≤12)}, acyl_{(C≤12)}, substituted acyl_{(C≤12)}, or a monovalent amino protecting group;
W is a covalent bond or a polyvalent polymer having 2-21 connection points;
n is 1 to 20 provided that when W is a covalent bond then n is 1 and when W is a polyvalent polymer then n is less than or equal to one less than the number of connection points;
Each X is independently a covalent bond, alkanediyl_{(C≤12)}, substituted alkanediyl_{(C≤12)}, one or more amino acid residues, or an oligomeric peptide;
Each X₂ is independently alkanediyl_{(C≤12)} or substituted alkanediyl_{(C≤12)};
Each R₃ is independently hydroxy, or amino; or
   alkoxy_{(C≤12)}, acyloxy_{(C≤12)}, alkylamino_{(C≤12)}, dialkylamino_{(C≤12)}, amido_{(C≤12)}, heteroaryl_{(C≤12)}, or a substituted version of any of these groups; or
   —X₆—C(O)R₁₂, wherein:
      X₆ is O, —NR_{b}—, or a covalent bond;
      R_{b} is hydrogen, alkyl_{(C≤6)}, substituted alkyl_{(C≤6)}, or a monovalent amino protecting group;
      R₁₂ is hydroxy or amino; or
      alkoxy_{(C≤12)}, alkylamino_{(C≤12)}, dialkylamino_{(C≤12)}, or a substituted version of any of these groups; or
   -A₃SO₂NR₁₃R₁₃′, -A₃P(O)(OH)OR₁₄, or -A₃SO₂OR₁₄′, wherein:
      A₃ is O, —NR_{c}—, or a covalent bond;
      R_{c} is hydrogen, alkyl_{(C≤6)}, substituted alkyl_{(C≤6)}, aryl_{(C≤12)}, heteroaryl_{(C≤12)}, aralkyl_{(C≤12)}, heteroaralkyl_{(C≤12)}, or a monovalent amino protecting group;
      R₁₃, R₁₃′, R₁₄, and R₁₄′ are each independently hydrogen, alkyl_{(C≤12)}, cycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups;
m is 0 or 1;
Each R₄ is independently the side chain moiety of glycine or valine; and
Each R₄′ is hydrogen, alkyl_{(C≤12)}, substituted alkyl_{(C≤12)}, aryl_{(C≤12)}, heteroaryl_{(C≤12)}, aralkyl_{(C≤12)}, heteroaralkyl_{(C≤12)}, acyl_{(C≤12)}, substituted acyl_{(C≤12)}, or a monovalent amino protecting group;
Each R₅ is independently the side chain moiety of glycine, alanine, ornithine, lysine, arginine, citrulline, asparagine, glutamine, or an amino-protected version thereof;
Each R₅′ is hydrogen, alkyl_{(C≤12)}, substituted alkyl_{(C≤12)}, aryl_{(C≤12)}, heteroaryl_{(C≤12)}, aralkyl_{(C≤12)}, heteroaralkyl_{(C≤12)}, acyl_{(C≤12)}, substituted acyl_{(C≤12)}, or a monovalent amino protecting group;
Each Q is independently a group of the formula:

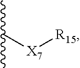

wherein:
   Each R₁₅ is independently hydrogen, —R₁₆, or —C(O)—R₁₆, wherein:
      R₁₆ is a therapeutic agent or an imaging agent;
   Each X₇ is independently a covalent bond, O, S, —NH—, alkanediyl_{(C≤12)}, substituted alkanediyl_{(C≤12)}, —(OCH₂CH₂)ₚ—, or substituted —(OCH₂CH₂)ₚ—, wherein:
      p is 0-50; or
      a group of the formula:

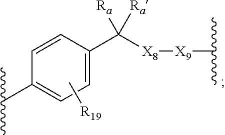

wherein:
Rₐ and Rₐ′ are each independently hydrogen, alkyl_{(C≤12)}, substituted alkyl_{(C≤12)}, aryl_{(C≤12)}, or heteroaryl_{(C≤12)};
X₈ is O or —NR₁₇R₁₇′—, wherein:
R₁₇ and R₁₇′ are each independently alkyl_{(C≤12)} or substituted alkyl_{(C≤12)};
R₁₉ hydrogen, sugar or a sugar derivative; and
X₉ is a covalent bond, O, S, —NH—, —(OCH₂CH₂)_{q}—, or substituted —(OCH₂CH₂)_{q}—, wherein:
q is 0-50; or
a group of the formula:

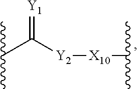

wherein:
Y₁ is O or S;
Y₂ is a covalent bond, O, S, —NH—, or —NR₁₈—, wherein:
R₁₈ is alkyl_{(C≤12)} or substituted alkyl_{(C≤12)}; and
X₁₀ is a covalent bond, alkyl_{(C≤12)}, substituted alkyl_{(C≤12)}, O, S, —NH—, —(CH₂CH₂O)ᵣ—, substituted —(CH₂CH₂O)ᵣ—, —(CH₂CH₂NR₂₀)ᵣ—, or substituted —(CH₂CH₂NR₂₀)ᵣ—;
r is 0-50;
R₂₀ is hydrogen, alkyl_{(C≤12)}, or substituted alkyl_{(C≤12)};
provided that when R₄ is valine, then R₄′ is not hydrogen;
or a pharmaceutically acceptable salt thereof.

In some embodiments, the compounds are further defined as:

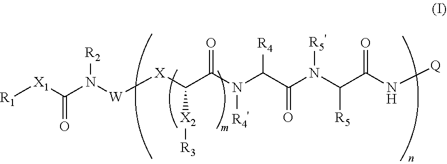

wherein:
   X₁ is a covalent bond, alkanediyl_{(C≤12)}, or substituted alkanediyl_{(C≤12)};
   R₁ is hydrogen, —ZR₆, —(OCH₂CH₂)ₙZR₆, or substituted —(OCH₂CH₂)ₙZR₆, wherein:
      n is 0-50; and
      R₆ is hydrogen, hydroxy, aminohydroxy, amino, mercapto, hydroxylamino, hydrazino, or azide; or
         alkyl_{(C≤12)}, alkenyl_{(C≤12)}, alkynyl_{(C≤12)}, alkylhydrazine_{(C≤12)}, or a substituted version of any of these groups;
         a polyglycine comprising from 1 to 6 glycine units; or
         a substructure of the formula:

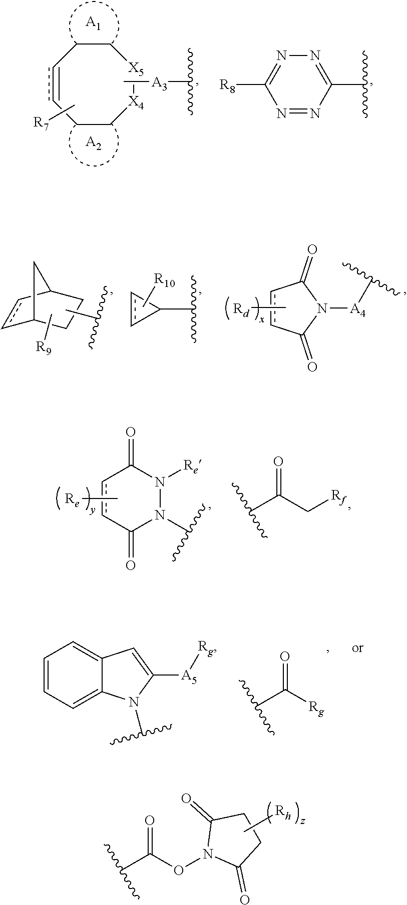

wherein:
A₁ and A₂ are each independently absent or arenediyl_{(C≤12)}, substituted arenediyl_{(C≤12)}, heteroarenediyl_{(C≤12)}, or substituted heteroarenediyl_{(C≤12)}, and form a fused arene_{(C≤12)}, substituted arene_{(C≤12)}, heteroarene_{(C≤12)}, or substituted heteroarene_{(C≤12)};
A₃ is a covalent bond, O, alkanediyl_{(C≤8)}, substituted alkanediyl_{(C≤8)}, alkoxydiyl_{(C≤8)}, substituted alkoxydiyl_{(C≤8)}, A₄ or A₅ are each independently selected from a covalent bond, alkanediyl_{(C≤8)}, substituted alkanediyl_{(C≤8)}, arenediyl_{(C≤8)}, or substituted arenediyl_{(C≤8)};
R_{d}, Rₑ, Rₑ′, and Rₕ are each independently selected from hydrogen, halo, thioether, selenoether, sulfate, tosylate, mesylate, aryl_{(C≤8)}, or substituted aryl_{(C≤8)};
R_{f} is halo;
R_{g} is amine, hydrazine, alkylamino_{(C≤8)}, substituted alkylamino_{(C≤8)}, dialkylamino_{(C≤8)}, substituted dialkylamino_{(C≤8)}, alkylhydrazine_{(C≤8)}, or substituted alkylhydrazine_{(C≤8)};
X₄ and X₅ are each independently O, N, C(O), CH₂, or X₄ and X₅ are alkanediyl_{(C≤8)} or substituted alkanediyl_{(C≤8)} and are taken together to form a fused cycloalkane group consisting of 3 to 8 ring atoms;
R₇ is hydrogen, hydroxy, amino, or oxo;
R₈ is carboxy; or
alkyl_{(C≤12)}, amido_{(C≤12)}, aryl_{(C≤12)}, heteroaryl_{(C≤12)}, —C(O)OR₁₁, —C(O)NR₁₁R₁₁′, or a substituted version of any of these groups wherein:
R₁₁ and R₁₁′ are each independently hydrogen; or
alkyl_{(C≤12)}, aryl_{(C≤12)}, or a substituted version of either of these groups;
R₉ and R₁₀ are each independently hydroxy, amino, halo; or
alkyl_{(C≤12)}, aryl_{(C≤12)}, or a substituted version of either of these groups;
Z is a covalent bond, alkanediyl_{(C≤12)}, —C(O)-alkanediyl_{(C≤12)}, —C(O)-alkanediyl_{(C≤12)}-C(O)NH—, or a substituted version of any of these groups;
R₂ is hydrogen, alkyl_{(C≤12)}, substituted alkyl_{(C≤12)}, acyl_{(C≤12)}, substituted acyl_{(C≤12)}, or a monovalent amino protecting group;
W is a covalent bond or a polyvalent polymer having 2-21 connection points;
n is 1 to 20 provided that when W is a covalent bond then n is 1 and when W is a polyvalent polymer then n is less than or equal to one less than the number of connection points;
Each X is independently a covalent bond, alkanediyl_{(C≤12)}, substituted alkanediyl_{(C≤12)}, one or more amino acid residues, or an oligomeric peptide;
Each X₂ is independently alkanediyl_{(C≤12)} or substituted alkanediyl_{(C≤12)};
Each R₃ is independently hydroxy, or amino; or
   alkoxy_{(C≤12)}, acyloxy_{(C≤12)}, alkylamino_{(C≤12)}, dialkylamino_{(C≤12)}, amido_{(C≤12)}, heteroaryl_{(C≤12)}, or a substituted version of any of these groups; or
   —X₆—C(O)R₁₂, wherein:
      X₆ is O, —NR_{b}—, or a covalent bond;
      R_{b} is hydrogen, alkyl_{(C≤6)}, substituted alkyl_{(C≤6)}, or a monovalent amino protecting group;
      R₁₂ is hydroxy or amino; or
      alkoxy_{(C≤12)}, alkylamino_{(C≤12)}, dialkylamino_{(C≤12)}, or a substituted version of any of these groups; or
   -A₃SO₂NR₁₃R₁₃′, -A₃P(O)(OH)OR₁₄, or -A₃SO₂OR₁₄′, wherein:
      A₃ is O, —NR_{c}—, or a covalent bond;
      R_{c} is hydrogen, alkyl_{(C≤6)}, substituted alkyl_{(C≤6)}, or a monovalent amino protecting group;
      R₁₃, R₁₃′, R₁₄, and R₁₄′ are each independently hydrogen, alkyl_{(C≤12)}, cycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups;
m is 0 or 1;
Each R₄ is independently the side chain moiety of glycine or valine; and
Each R₄′ is hydrogen, alkyl_{(C≤12)}, substituted alkyl_{(C≤12)}, acyl_{(C≤12)}, substituted acyl_{(C≤12)}, or a monovalent amino protecting group;
Each R₅ is independently the side chain moiety of glycine, alanine, ornithine, lysine, arginine, citrulline, asparagine, glutamine, or an amino-protected version thereof;
Each R₅′ is hydrogen, alkyl_{(C≤12)}, substituted alkyl_{(C≤12)}, acyl_{(C≤12)}, substituted acyl_{(C≤12)}, or a monovalent amino protecting group;
Each Q is independently a group of the formula:

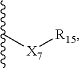

wherein:
   Each R₁₅ is independently hydrogen, —R₁₆, or —C(O)—R₁₆, wherein:
      R₁₆ is a therapeutic agent or an imaging agent;
   Each X₇ is independently a covalent bond, O, S, —NH—, alkanediyl_{(C≤12)}, substituted alkanediyl_{(C≤12)}, —(OCH₂CH₂)ₚ—, or substituted —(OCH₂CH₂)ₚ—, wherein:
      p is 0-50; or
      a group of the formula:

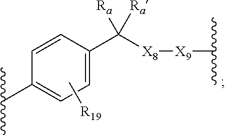

wherein:
Rₐ and Rₐ′ are each independently hydrogen, alkyl_{(C≤12)}, or substituted alkyl_{(C≤12)};
X₈ is O or —NR₁₇R₁₇′—, wherein:
R₁₇ and R₁₇′ are each independently alkyl_{(C≤12)} or substituted alkyl_{(C≤12)};
R₁₉ hydrogen, sugar or a sugar derivative; and
X₉ is a covalent bond, O, S, —NH—, —(OCH₂CH₂)_{q}—, or substituted —(OCH₂CH₂)_{q}—, wherein:
q is 0-50; or
a group of the formula:

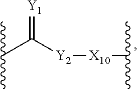

wherein:
Y₁ is O or S;
Y₂ is O, S, —NH—, or —NR₁₈—, wherein:
R₁₈ is alkyl_{(C≤12)} or substituted alkyl_{(C≤12)}; and
X₁₀ is a covalent bond, alkyl_{(C≤12)}, substituted alkyl_{(C≤12)}, O, S, —NH—, —(CH₂CH₂O)ᵣ—, substituted —(CH₂CH₂O)ᵣ—, —(CH₂CH₂NR₂₀)ᵣ—, or substituted —(CH₂CH₂NR₂₀)ᵣ—;
r is 0-50;
R₂₀ is hydrogen, alkyl_{(C≤12)}, or substituted alkyl_{(C≤12)};
provided that when R₄ is valine, then R₄′ is not hydrogen;
or a pharmaceutically acceptable salt thereof.

In some embodiments, the compounds are further defined as:

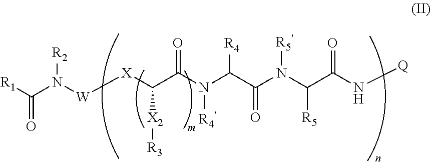

wherein:
   R₁ is hydrogen, alkyl_{(C≤12)}, substituted alkyl_{(C≤12)}, —ZR₆, —(OCH₂CH₂)ₙZR₆, or substituted —(OCH₂CH₂)ₙZR₆, wherein:
      n is 0-50; and
      R₆ is hydrogen, hydroxy, aminohydroxy, amino, mercapto, hydroxylamino, hydrazino, or azide; or
         alkyl_{(C≤12)}, alkenyl_{(C≤12)}, alkynyl_{(C≤12)}, alkylhydrazine_{(C≤12)}, or a substituted version of any of these groups;
         a polyglycine comprising from 1 to 6 glycine units; or
         a substructure of the formula:

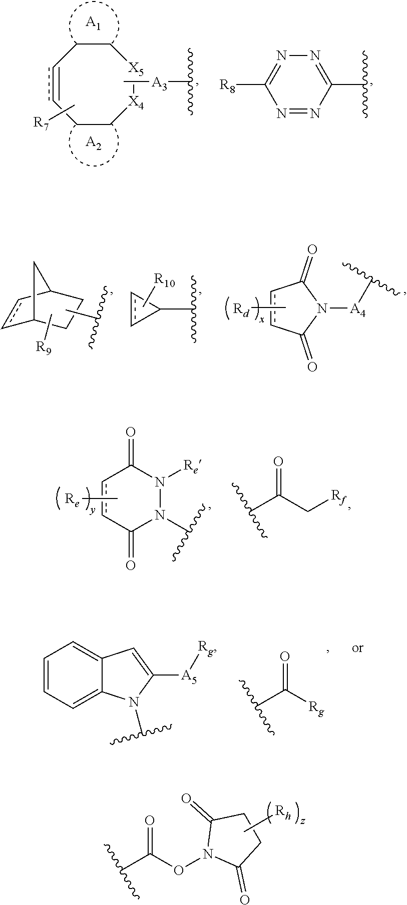

wherein:
A₁ and A₂ are each independently absent or arenediyl_{(C≤12)}, substituted arenediyl_{(C≤12)}, heteroarenediyl_{(C≤12)}, or substituted heteroarenediyl_{(C≤12)}, and form a fused arene_{(C≤12)}, substituted arene_{(C≤12)}, heteroarene_{(C≤12)}, or substituted heteroarene_{(C≤12)};
A₃ is a covalent bond, O, alkanediyl_{(C≤8)}, substituted alkanediyl_{(C≤8)}, alkoxydiyl_{(C≤8)}, substituted alkoxydiyl_{(C≤8)}, A₄ or A₅ are each independently selected from a covalent bond, alkanediyl_{(C≤8)}, substituted alkanediyl_{(C≤8)}, arenediyl_{(C≤8)}, or substituted arenediyl_{(C≤8)};
R_{d}, Rₑ, Rₑ′, and Rₕ are each independently selected from hydrogen, halo, thioether, selenoether, sulfate, tosylate, mesylate, aryl_{(C≤8)}, or substituted aryl_{(C≤8)};
R_{f} is halo;
R_{g} is amine, hydrazine, alkylamino_{(C≤8)}, substituted alkylamino_{(C≤8)}, dialkylamino_{(C≤8)}, substituted dialkylamino_{(C≤8)}, alkylhydrazine_{(C≤8)}, or substituted alkylhydrazine_{(C≤8)};
X₄ and X₅ are each independently O, N, C(O), CH₂, or X₄ and X₅ are alkanediyl_{(C≤8)} or substituted alkanediyl_{(C≤8)} and are taken together to form a fused cycloalkane group consisting of 3 to 8 ring atoms;
R₇ is hydrogen, hydroxy, amino, or oxo;
R₈ is carboxy; or
alkyl_{(C≤12)}, amido_{(C≤12)}, aryl_{(C≤12)}, heteroaryl_{(C≤12)}, —C(O)OR₁₁, —C(O)NR₁₁R₁₁′, or a substituted version of any of these groups wherein:
R₁₁ and R₁₁′ are each independently hydrogen; or
alkyl_{(C≤12)}, aryl_{(C≤12)}, or a substituted version of either of these groups;
R₉ and R₁₀ are each independently hydroxy, amino, halo; or
alkyl_{(C≤12)}, aryl_{(C≤12)}, or a substituted version of either of these groups;
Z is a covalent bond, alkanediyl_{(C≤12)}, —C(O)-alkanediyl_{(C≤12)}, —C(O)-alkanediyl_{(C≤12)}-C(O)NH—, or a substituted version of any of these groups;
R₂ is hydrogen, alkyl_{(C≤12)}, substituted alkyl_{(C≤12)}, acyl_{(C≤12)}, substituted acyl_{(C≤12)}, or a monovalent amino protecting group;
W is a covalent bond or a polyvalent polymer having 2-21 connection points;
n is 1 to 20 provided that when W is a covalent bond then n is 1 and when W is a polyvalent polymer then n is less than or equal to one less than the number of connection points;
Each X is independently a covalent bond, alkanediyl_{(C≤12)}, substituted alkanediyl_{(C≤12)}, one or more amino acid residues, or oligomeric peptide;
Each X₂ is independently alkanediyl_{(C≤12)} or substituted alkanediyl_{(C≤12)};
Each R₃ is independently hydroxy, or amino; or
   alkoxy_{(C≤12)}, acyloxy_{(C≤12)}, alkylamino_{(C≤12)}, dialkylamino_{(C≤12)}, amido_{(C≤12)} heteroaryl_{(C≤12)}, or a substituted version of any of these groups; or
   —X₆—C(O)R₁₂, wherein:
      X₆ is O, —NR_{b}—, or a covalent bond;
      R_{b} is hydrogen, alkyl_{(C≤6)}, substituted alkyl_{(C≤6)}, or a monovalent amino protecting group;
      R₁₂ is hydroxy or amino; or
      alkoxy_{(C≤12)}, alkylamino_{(C≤12)}, dialkylamino_{(C≤12)}, or a substituted version of any of these groups; or
   A₃SO₂NR₁₃R₁₃′, -A₃P(O)(OH)OR₁₄, or -A₃SO₂OR₁₄′, wherein:
      A₃ is O, —NR_{c}—, or a covalent bond;
      R_{c} is hydrogen, alkyl_{(C≤6)}, substituted alkyl_{(C≤6)}, or a monovalent amino protecting group;
      R₁₃, R₁₃′, R₁₄, and R₁₄′ are each independently hydrogen, alkyl_{(C≤12)}, cycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups;
m is 0 or 1;
Each R₄ is independently the side chain moiety of glycine or valine; and
Each R₄′ is hydrogen, alkyl_{(C≤12)}, substituted alkyl_{(C≤12)}, acyl_{(C≤12)}, substituted acyl_{(C≤12)}, or a monovalent amino protecting group;
Each R₅ is independently the side chain moiety of glycine, alanine, ornithine, lysine, arginine, citrulline, asparagine, glutamine, or an amino-protected version thereof;
Each R₅′ is hydrogen, alkyl_{(C≤12)}, substituted alkyl_{(C≤12)}, acyl_{(C≤12)}, substituted acyl_{(C≤12)}, or a monovalent amino protecting group;
Each Q is independently a group of the formula:

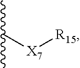

wherein:
   Each R₁₅ is independently hydrogen, —R₁₆, or —C(O)—R₁₆, wherein:
      R₁₆ is a therapeutic agent or an imaging agent;
   Each X₇ is independently a covalent bond, O, S, —NH—, alkanediyl_{(C≤12)}, substituted alkanediyl_{(C≤12)}, —(OCH₂CH₂)ₚ—, or substituted —(OCH₂CH₂)ₚ—, wherein:
      p is 0-50; or
      a group of the formula:

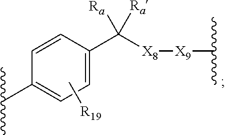

wherein:
Rₐ and Rₐ′ are each independently hydrogen, alkyl_{(C≤12)}, or substituted alkyl_{(C≤12)};
X₈ is O or —NR₁₇R₁₇′—, wherein:
R₁₇ and R₁₇′ are each independently alkyl_{(C≤12)} or substituted alkyl_{(C≤12)};
R₁₉ hydrogen, sugar or a sugar derivative; and
X₉ is a covalent bond, O, S, —NH—, —(OCH₂CH₂)_{q}, or substituted —(OCH₂CH₂)_{q}, wherein:
q is 0-50; or
a group of the formula:

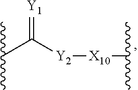

wherein:
Y₁ is O or S;
Y₂ is O, S, —NH—, or —NR₁₈—, wherein:
R₁₈ is alkyl_{(C≤12)} or substituted alkyl_{(C≤12)}; and
X₁₀ is a covalent bond, alkyl_{(C≤12)}, substituted alkyl_{(C≤12)}, O, S, —NH—, —(CH₂CH₂O)ᵣ—, substituted —(CH₂CH₂O)ᵣ—, —(CH₂CH₂NR₂₀)ᵣ—, or substituted —(CH₂CH₂NR₂₀)ᵣ—;
r is 0-50;
R₂₀ is hydrogen, alkyl_{(C≤12)}, or substituted alkyl_{(C≤12)};
or a pharmaceutically acceptable salt thereof.

In some embodiments, the compounds are further defined as:

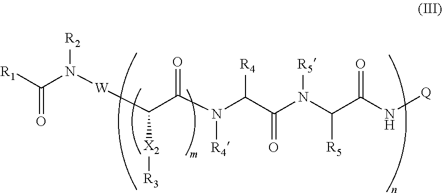

wherein:
   R₁ is hydrogen, alkyl_{(C≤12)}, substituted alkyl_{(C≤12)}, —ZR₆, —(OCH₂CH₂)ₙZR₆, or substituted —(OCH₂CH₂)ₙZR₆, wherein:
      n is 0-50; and
      R₆ is hydrogen, hydroxy, aminohydroxy, amino, mercapto, hydroxylamino, hydrazino, or azide; or
         alkyl_{(C≤12)}, alkenyl_{(C≤12)}, alkynyl_{(C≤12)}, alkylhydrazine_{(C≤12)}, or a substituted version of any of these groups;
         a polyglycine comprising from 1 to 6 glycine units; or
         a substructure of the formula:

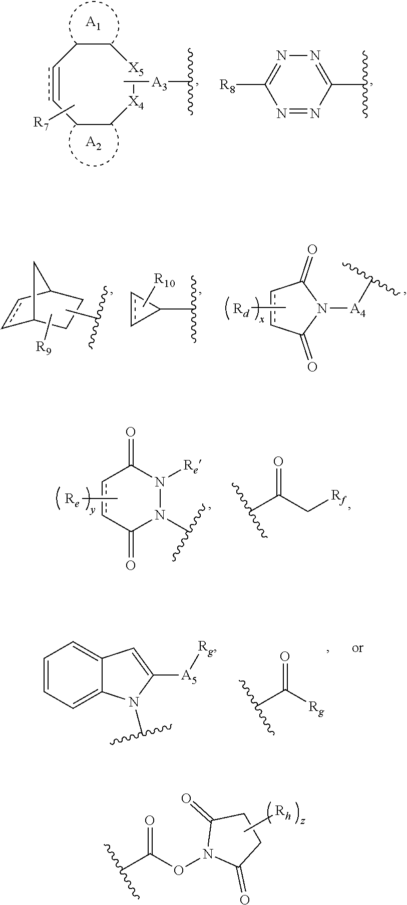

wherein:
A₁ and A₂ are each independently absent or arenediyl_{(C≤12)}, substituted arenediyl_{(C≤12)}, heteroarenediyl_{(C≤12)}, or substituted heteroarenediyl_{(C≤12)}, and form a fused arene_{(C≤12)}, substituted arene_{(C≤12)}, heteroarene_{(C≤12)}, or substituted heteroarene_{(C≤12)};
A₃ is a covalent bond, O, alkanediyl_{(C≤8)}, substituted alkanediyl_{(C≤8)}, alkoxydiyl_{(C≤8)}, substituted alkoxydiyl_{(C≤8)}, A₄ or A₅ are each independently selected from a covalent bond, alkanediyl_{(C≤8)}, substituted alkanediyl_{(C≤8)}, arenediyl_{(C≤8)}, or substituted arenediyl_{(C≤8)};
R_{d}, Rₑ, Rₑ′, and Rₕ are each independently selected from hydrogen, halo, thioether, selenoether, sulfate, tosylate, mesylate, aryl_{(C≤8)}, or substituted aryl_{(C≤8)};
R_{f} is halo;
R_{g} is amine, hydrazine, alkylamino_{(C≤8)}, substituted alkylamino_{(C≤8)}, dialkylamino_{(C≤8)}, substituted dialkylamino_{(C≤8)}, alkylhydrazine_{(C≤8)}, or substituted alkylhydrazine_{(C≤8)};
X₄ and X₅ are each independently O, N, C(O), CH₂, or X₄ and X₅ are alkanediyl_{(C≤8)} or substituted alkanediyl_{(C≤8)} and are taken together to form a fused cycloalkane group consisting of 3 to 8 ring atoms;
R₇ is hydrogen, hydroxy, amino, or oxo;
R₈ is carboxy; or
alkyl_{(C≤12)}, amido_{(C≤12)}, aryl_{(C≤12)}, heteroaryl_{(C≤12)}; —C(O)OR₁₁, —C(O)NR₁₁R₁₁′, or a substituted version of any of these groups wherein:
R₁₁ and R₁₁′ are each independently hydrogen; or
alkyl_{(C≤12)}, aryl_{(C≤12)}, or a substituted version of either of these groups;
R₉ and R₁₀ are each independently hydroxy, amino, halo; or
alkyl_{(C≤12)}, aryl_{(C≤12)}, or a substituted version of either of these groups;
Z is a covalent bond, alkanediyl_{(C≤12)}, —C(O)-alkanediyl_{(C≤12)}, —C(O)-alkanediyl_{(C≤12)}-C(O)NH—, or a substituted version of any of these groups;
R₂ is hydrogen, alkyl_{(C≤12)}, substituted alkyl_{(C≤12)}, acyl_{(C≤12)}, substituted acyl_{(C≤12)}, or a monovalent amino protecting group;
W is a covalent bond or a polyvalent polymer having 2-21 connection points;
n is 1 to 20 provided that when W is a covalent bond then n is 1 and when W is a polyvalent polymer then n is less than or equal to one less than the number of connection points;
Each X₂ is independently alkanediyl_{(C≤12)} or substituted alkanediyl_{(C≤12)};
Each R₃ is independently hydroxy, or amino; or
   alkoxy_{(C≤12)}, acyloxy_{(C≤12)}, alkylamino_{(C≤12)}, dialkylamino_{(C≤12)}, amido_{(C≤12)} heteroaryl_{(C≤12)}, or a substituted version of any of these groups; or
   —X₆—C(O)R₁₂, wherein:
      X₆ is O, —NR_{b}—, or a covalent bond;
      R_{b} is hydrogen, alkyl_{(C≤6)}, substituted alkyl_{(C≤6)}, or a monovalent amino protecting group;
      R₁₂ is hydroxy or amino; or
      alkoxy_{(C≤12)}, alkylamino_{(C≤12)}, dialkylamino_{(C≤12)}, or a substituted version of any of these groups; or
   -A₃SO₂NR₁₃R₁₃′, -A₃P(O)(OH)OR₁₄, or -A₃SO₂OR₁₄′, wherein:
      A₃ is O, —NR_{c}—, or a covalent bond;
      R_{c} is hydrogen, alkyl_{(C≤6)}, substituted alkyl_{(C≤6)}, or a monovalent amino protecting group;
      R₁₃, R₁₃′, R₁₄, and R₁₄′ are each independently hydrogen, alkyl_{(C≤12)}, cycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups;
m is 0 or 1;
Each R₄ is independently the side chain moiety of glycine or valine; and
Each R₄′ is hydrogen, alkyl_{(C≤12)}, substituted alkyl_{(C≤12)}, acyl_{(C≤12)}, substituted acyl_{(C≤12)}, or a monovalent amino protecting group;
Each R₅ is independently the side chain moiety of glycine, alanine, ornithine, lysine, arginine, citrulline, asparagine, glutamine, or an amino-protected version thereof;
Each R₅′ is hydrogen, alkyl_{(C≤12)}, substituted alkyl_{(C≤12)}, acyl_{(C≤12)}, substituted acyl_{(C≤12)}, or a monovalent amino protecting group;
Each Q is independently a group of the formula:

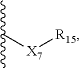

wherein:
   Each R₁₅ is independently hydrogen, —R₁₆, or —C(O)—R₁₆, wherein:
      R₁₆ is a therapeutic agent or an imaging agent;
   Each X₇ is independently a covalent bond, O, S, —NH—, alkanediyl_{(C≤12)}, substituted alkanediyl_{(C≤12)}, —(OCH₂CH₂)ₚ—, or substituted —(OCH₂CH₂)ₚ—, wherein:
      p is 0-50; or
      a group of the formula:

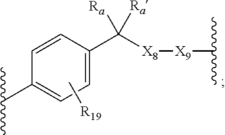

wherein:
Rₐ and Rₐ′ are each independently hydrogen, alkyl_{(C≤12)}, or substituted alkyl_{(C≤12)};
X₈ is O or —NR₁₇R₁₇′—, wherein:
R₁₇ and R₁₇′ are each independently alkyl_{(C≤12)} or substituted alkyl_{(C≤12)};
R₁₉ hydrogen, sugar or a sugar derivative; and
X₉ is a covalent bond, O, S, —NH—, —(OCH₂CH₂)_{q}, or substituted —(OCH₂CH₂)_{q}, wherein:
q is 0-50; or
a group of the formula:

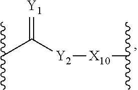

wherein:
Y₁ is O or S;
Y₂ is O, S, —NH—, or —NR₁₈—, wherein:
R₁₈ is alkyl_{(C≤12)} or substituted alkyl_{(C≤12)}; and
X₁₀ is a covalent bond, alkyl_{(C≤12)}, substituted alkyl_{(C≤12)}, O, S, —NH—, —(CH₂CH₂O)ᵣ—, substituted —(CH₂CH₂O)ᵣ—, —(CH₂CH₂NR₂₀)ᵣ—, or substituted —(CH₂CH₂NR₂₀)ᵣ—;
r is 0-50;
R₂₀ is hydrogen, alkyl_{(C≤12)}, or substituted alkyl_{(C≤12)};
or a pharmaceutically acceptable salt thereof.

In some embodiments, the compounds are further defined as:

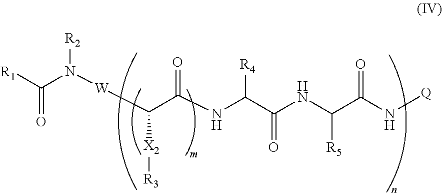

wherein:
   R₁ is hydrogen, alkyl_{(C≤12)}, substituted alkyl_{(C≤12)}, —ZR₆, —(OCH₂CH₂)ₙZR₆, or substituted —(OCH₂CH₂)ₙZR₆, wherein:
      n is 0-50; and
      R₆ is hydrogen, hydroxy, aminohydroxy, amino, mercapto, hydroxylamino, hydrazino, or azide; or
         alkyl_{(C≤12)}, alkenyl_{(C≤12)}, alkynyl_{(C≤12)}, alkylhydrazine_{(C≤12)}, or a substituted version of any of these groups;
         a polyglycine comprising from 1 to 6 glycine units; or
         a substructure of the formula:

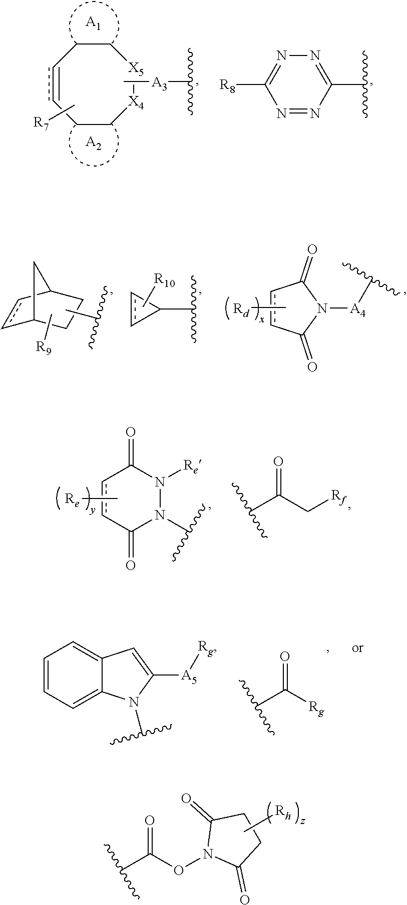

wherein:
A₁ and A₂ are each independently absent or arenediyl_{(C≤12)}, substituted arenediyl_{(C≤12)}, heteroarenediyl_{(C≤12)}, or substituted heteroarenediyl_{(C≤12)}, and form a fused arene_{(C≤12)}, substituted arene_{(C≤12)}, heteroarene_{(C≤12)}, or substituted heteroarene_{(C≤12)};
A₃ is a covalent bond, O, alkanediyl_{(C≤8)}, substituted alkanediyl_{(C≤8)}, alkoxydiyl_{(C≤8)}, substituted alkoxydiyl_{(C≤8)},
A₄ or A₅ are each independently selected from a covalent bond, alkanediyl_{(C≤8)}, substituted alkanediyl_{(C≤8)}, arenediyl_{(C≤8)}, or substituted arenediyl_{(C≤8)};
R_{d}, Rₑ, Rₑ′, and Rₕ are each independently selected from hydrogen, halo, thioether, selenoether, sulfate, tosylate, mesylate, aryl_{(C≤8)}, or substituted aryl_{(C≤8)};
R_{f} is halo;
R_{g} is amine, hydrazine, alkylamino_{(C≤8)}, substituted alkylamino_{(C≤8)}, dialkylamino_{(C≤8)}, substituted dialkylamino_{(C≤8)}, alkylhydrazine_{(C≤8)}, or substituted alkylhydrazine_{(C≤8)};
X₄ and X₅ are each independently O, N, C(O), CH₂, or X₄ and X₅ are alkanediyl_{(C≤8)} or substituted alkanediyl_{(C≤8)} and are taken together to form a fused cycloalkane group consisting of 3 to 8 ring atoms;
R₇ is hydrogen, hydroxy, amino, or oxo;
R₈ is carboxy; or
alkyl_{(C≤12)}, amido_{(C≤12)}, aryl_{(C≤12)}, heteroaryl_{(C≤12)}; —C(O)OR₁₁, —C(O)NR₁₁R₁₁′, or a substituted version of any of these groups wherein:
R₁₁ and R₁₁′ are each independently hydrogen; or
alkyl_{(C≤12)}, aryl_{(C≤12)}, or a substituted version of either of these groups;
R₉ and R₁₀ are each independently hydroxy, amino, halo; or
alkyl_{(C≤12)}, aryl_{(C≤12)}, or a substituted version of either of these groups;
Z is a covalent bond, alkanediyl_{(C≤12)}, —C(O)-alkanediyl_{(C≤12)}, —C(O)-alkanediyl_{(C≤12)}—C(O)NH—, or a substituted version of any of these groups;
R₂ is hydrogen, alkyl_{(C≤12)}, substituted alkyl_{(C≤12)}, acyl_{(C≤12)}, substituted acyl_{(C≤12)}, or a monovalent amino protecting group;
W is a covalent bond or a polyvalent polymer having 2-21 connection points;
n is 1 to 20 provided that when W is a covalent bond then n is 1 and when W is a polyvalent polymer then n is less than or equal to one less than the number of connection points;
Each X₂ is independently alkanediyl_{(C≤12)} or substituted alkanediyl_{(C≤12)};
Each R₃ is independently hydroxy, or amino; or
   alkoxy_{(C≤12)}, acyloxy_{(C≤12)}, alkylamino_{(C≤12)}, dialkylamino_{(C≤12)}, amido_{(C≤12)} heteroaryl_{(C≤12)}, or a substituted version of any of these groups; or
   —X₆—C(O)R₁₂, wherein:
      X₆ is O, —NR_{b}—, or a covalent bond;
      R_{b} is hydrogen, alkyl_{(C≤6)}, substituted alkyl_{(C≤6)}, or a monovalent amino protecting group;
      R₁₂ is hydroxy or amino; or
      alkoxy_{(C≤12)}, alkylamino_{(C≤12)}, dialkylamino_{(C≤12)}, or a substituted version of any of these groups; or
   -A₃SO₂NR₁₃R₁₃′, -A₃P(O)(OH)OR₁₄, or -A₃SO₂OR₁₄′, wherein:
      A₃ is O, —NR_{c}—, or a covalent bond;
      R_{c} is hydrogen, alkyl_{(C≤6)}, substituted alkyl_{(C≤6)}, or a monovalent amino protecting group;
      R₁₃, R₁₃′, R₁₄, and R₁₄′ are each independently hydrogen, alkyl_{(C≤12)}, cycloalkyl_{(C≤12)}, aryl_{(C≤12)}, heteroaryl_{(C≤12)}, aralkyl_{(C≤12)}, heteroaralkyl_{(C≤12)}, or a substituted version of any of these groups;
m is 0 or 1;
Each R₄ is independently the side chain moiety of glycine or valine;
Each R₅ is independently the side chain moiety of glycine, alanine, ornithine, lysine, arginine, citrulline, asparagine, glutamine, or an amino-protected version thereof;
Each Q is independently a group of the formula:

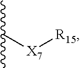

wherein:
   Each R₁₅ is independently hydrogen, —R₁₆, or —C(O)—R₁₆, wherein:
      R₁₆ is a therapeutic agent or an imaging agent;
   Each X₇ is independently a covalent bond, O, S, —NH—, alkanediyl_{(C≤12)}, substituted alkanediyl_{(C≤12)}, —(OCH₂CH₂)ₚ—, or substituted —(OCH₂CH₂)ₚ—, wherein:
      p is 0-50; or
      a group of the formula:

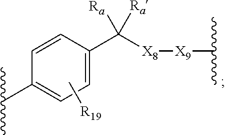

wherein:
Rₐ and Rₐ′ are each independently hydrogen, alkyl_{(C≤12)}, or substituted alkyl_{(C≤12)};
X₈ is O or —NR₁₇R₁₇′—, wherein:
R₁₇ and R₁₇′ are each independently alkyl_{(C≤12)} or substituted alkyl_{(C≤12)};
R₁₉ hydrogen, sugar or a sugar derivative; and
X₉ is a covalent bond, O, S, —NH—, —(OCH₂CH₂)_{q}—, or substituted —(OCH₂CH₂)_{q}, wherein:
q is 0-50; or
a group of the formula:

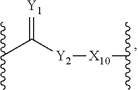

wherein:
Y₁ is O or S;
Y₂ is O, S, —NH—, or —NR₁₈—, wherein:
R₁₈ is alkyl_{(C≤12)} or substituted alkyl_{(C≤12)}; and
X₁₀ is a covalent bond, alkyl_{(C≤12)}, substituted alkyl_{(C≤12)}, O, S, —NH—, —(CH₂CH₂O)ᵣ—, substituted —(CH₂CH₂O)ᵣ—, —(CH₂CH₂NR₂₀)ᵣ—, or substituted —(CH₂CH₂NR₂₀)ᵣ—;
r is 0-50;
R₂₀ is hydrogen, alkyl_{(C≤12)}, or substituted alkyl_{(C≤12)};
or a pharmaceutically acceptable salt thereof.

In some embodiments, X₁ is a covalent bond. In some embodiments, X is a covalent bond. In some embodiments, R₄′ is hydrogen. In some embodiments, R₅′ is hydrogen.

In some embodiments, W is a covalent bond. In other embodiments, W is a polyvalent polymer with 1-5 connection points. In some embodiments, n is 1. In other embodiments, n is 2, 3, 4, or 5.

In some embodiments, X₂ is alkanediyl_{(C≤12)} such as —CH₂CH₂—. In some embodiments, R₃ is —X₆—C(O)R₁₂, wherein: X₆ is O, —NR_{b}—, or a covalent bond; R_{b} is hydrogen, alkyl_{(C≤6)}, substituted alkyl_{(C≤6)}, or a monovalent amino protecting group; R₁₂ is hydroxy or amino; or alkoxy_{(C≤12)}, alkylamino_{(C≤12)}, dialkylamino_{(C≤12)}, or a substituted version of any of these groups. In some embodiments, X₆ is a covalent bond. In some embodiments, R₁₂ is hydroxy.

In some embodiments, m is 0. In other embodiments, m is 1. In some embodiments, each R₄ is the side chain or glycine. In some embodiments, each R₄ is the side chain of valine.

In some embodiments, each R₅ is the side chain of citrulline.

In some embodiments, Q is:

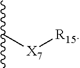

In some embodiments, X₇ is a group of the formula:

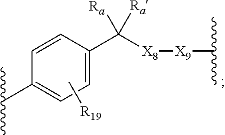

wherein:
   Rₐ and Rₐ′ are each independently hydrogen, alkyl_{(C≤12)}, or substituted alkyl_{(C≤12)};
   X₈ is O or —NR₁₇R₁₇′—, wherein:
      R₁₇ and R₁₇′ are each independently alkyl_{(C≤12)} or substituted alkyl_{(C≤12)};
   R₁₉ hydrogen, sugar or a sugar derivative; and
   X₉ is a covalent bond, O, S, —NH—, —(OCH₂CH₂)_{q}, or substituted —(OCH₂CH₂)_{q}—, wherein:
      q is 0-50; or
      a group of the formula:

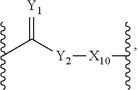

wherein:
   Y₁ is O or S;
   Y₂ is O, S, —NH—, or —NR₁₈—, wherein:
   R₁₈ is alkyl_{(C≤12)} or substituted alkyl_{(C≤12)}; and
   X₁₀ is a covalent bond, alkyl_{(C≤12)}, substituted alkyl_{(C≤12)}, O, S, —NH—, —(CH₂CH₂O)ᵣ—, substituted —(CH₂CH₂O)ᵣ—, —(CH₂CH₂NR₂₀)ᵣ—, or substituted —(CH₂CH₂NR₂₀)ᵣ—;
   r is 0-50;
   R₂₀ is hydrogen, alkyl_{(C≤12)}, or substituted alkyl_{(C≤12)}.

In some embodiments, Rₐ is hydrogen. In some embodiments, Rₐ′ is hydrogen. In some embodiments, R₁₉ is hydrogen. In some embodiments, X₈ is O. In some embodiments, X₉ is a group of the formula:

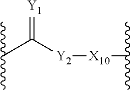

wherein:
   Y₁ is O or S;
   Y₂ is O, S, —NH—, or —NR₁₈—, wherein:
      R₁₈ is alkyl_{(C≤12)} or substituted alkyl_{(C≤12)}; and
   X₁₀ is a covalent bond, alkyl_{(C≤12)}, substituted alkyl_{(C≤12)}, O, S, —NH—, —(CH₂CH₂O)ᵣ—, substituted —(CH₂CH₂O)ᵣ—, —(CH₂CH₂NR₂₀)ᵣ—, or substituted —(CH₂CH₂NR₂₀)ᵣ—;
      r is 0-50;
      R₂₀ is hydrogen, alkyl_{(C≤12)}, or substituted alkyl_{(C≤12)}.

In some embodiments, Y₁ is O. In some embodiments, Y₂ is —NR₁₈—. In some embodiments, R₁₈ is alkyl_{(C≤12)} such as methyl. In some embodiments, X₁₀ is a covalent bond. In some embodiments, R₁₅ is —R₁₆ or —C(O)—R₁₆. In some embodiments, R₁₆ is a therapeutic drug. In some embodiments, the therapeutic drug is a chemotherapeutic drug. In other embodiments, R₁₆ is an imaging agent. In some embodiments, the imaging agent is a radioactive tracer. In other embodiments, the imaging agent is a fluorescent molecule. In some embodiments, R₁₅ is hydrogen.

In another aspect, the present disclosure provides drug conjugates comprising:
(A) a compound described herein;
(B) a linker; and
(C) a cell targeting group.

In some embodiments, the cell targeting group is an antibody. In other embodiments, the cell targeting group is an antibody fragment. In other embodiments, the cell targeting group is a protein. In other embodiments, the cell targeting group is a small molecule. In some embodiments, the small molecule is a receptor-specific ligand molecule. In some embodiments, the linker is a non-covalent bond formed by hydrogen bonding, nucleobase pairing, electrostatic interactions, pi stacking, van der Waals interactions, or dipole-dipole interactions. In some embodiments, the linker is a covalent bond. In other embodiments, the linker is a monovalent spacer comprising 1 connection point. In some embodiments, the linker is a polyvalent spacer comprising 2-21 connection points.

In some embodiments, the drug conjugate is formed by condensation, 1,3-dipolar cycloaddition reaction, Diels-Alder reaction, hetero-Diels-Alder reaction, Michael reaction, nucleophilic substitution reaction, non-aldol type carbonyl reaction, addition to carbon-carbon multiple bond, oxidation reaction, enzymatic amino acid or peptide modification, transpeptidation, and/or click reaction.

In some embodiments, the linker is a group, W, in the compound. In some embodiments, the linker is a group of the formula:

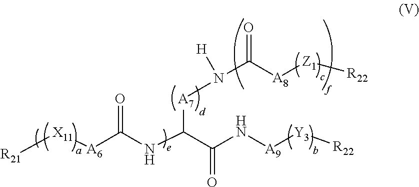

wherein:
   A₆, A₇, A₈, and A₉ are each independently alkanediyl_{C1-12}, arenediyl_{C1-12}, heteroarenediyl_{C1-12}, cycloalkanediyl_{C1-12}, heterocycloalkanediyl_{C1-12}, or a substituted version thereof, or a side chain group of a canonical amino acid;
   X₁₁, Y₃, and Z₁ are each independently a covalent bond, —[O(CH₂)_{g}]—, —[O(CHW₁′)_{q}]—, or —[O(CW₁′W₁″)_{q}]—;
   wherein:
      W₁′ and W₁″ are each independently amino, hydroxy, halo, mercapto, alkyl_{C1-12}, cycloalkyl_{C1-12}, alkenyl_{C1-12}, alkynyl_{C1-12}, aryl_{C1-12}, aralkyl_{C1-12}, heteroaryl_{C1-12}, heteroaralkyl_{C1-12}, heterocycloalkyl_{C1-12}, acyl_{C1-12}, acyloxy_{C1-12}, alkylamino_{C1-12}, or a substituted version of thereof;
      q is 1-3;
   a, b, c, and d are each independently 0-12;
   e and f are each independently 0, 1, 2, or 3;
   R₂₁, R₂₂, and R₂₃ are each independently hydrogen, —NH₂, —NHR₂₄, —NR₂₄R₂₅, —N₃, heteroaryl_{(C≤12)}, substituted heteroaryl_{(C≤12)}, -arenediyl_{(C1-12)}-heteroaryl_{(C≤12)}, substituted -arenediyl_{(C≤12)}-heteroaryl_{(C≤12)}, or a conjugating group;
   wherein:
      R₂₄ and R₂₅ are each independently alkyl_{C1-12}, cycloalkyl_{C1-12}, alkenyl_{C1-12}, alkynyl_{C1-12}, aryl_{C1-12}, aralkyl_{C1-12}, heteroaryl_{C1-12}, heteroaralkyl_{C1-12}, heterocycloalkyl_{C1-12}, acyl_{C1-12}, acyloxy_{C1-12}, alkylamino_{C1-12}, or a substituted version thereof, or a monovalent amino protecting group; or
      R₂₄ and R₂₅ are taken together and is a divalent amino protecting group;
   provided that at least one of R₂₁, R₂₂, and R₂₃ is —NH₂ or a group containing —NH₂ and at least one of R₂₁, R₂₂, and R₂₃ is —N₃, heteroaryl_{(C≤12)}, or -arenediyl_{(C≤12)}-heteroaryl_{(C≤12)};
or a pharmaceutically acceptable salt thereof.

In some embodiments, A₆ is alkanediyl_{C1-12} or substituted alkanediyl_{C1-12}. In some embodiments, A₆ is alkanediyl_{C1-12} such as CH₂. In some embodiments, A₈ is alkanediyl_{C1-12} or substituted alkanediyl_{C1-12}. In some embodiments, A₈ is alkanediyl_{C1-12} such as CH₂. In some embodiments, A₉ is alkanediyl_{C1-12} or substituted alkanediyl_{C1-12}. In some embodiments, A₉ is alkanediyl_{C1-12} such as CH₂CH₂. In some embodiments, A₇ is alkanediyl_{C1-12} or substituted alkanediyl_{C1-12}. In some embodiments, A₇ is alkanediyl_{C1-12} such as CH₂CH₂CH₂CH₂.

In some embodiments, X₁n is —[O(CH₂)_{q}]—. In some embodiments, Y₃ is —[O(CH₂)_{q}]—. In some embodiments, Z₁ is —[O(CH₂)_{q}]—. In some embodiments, q is 1, 2, or 3. In some embodiments, q is 2. In some embodiments, a is 2, 3, or 4. In some embodiments, a is 3. In some embodiments, b is 2, 3, or 4. In some embodiments, b is 3. In some embodiments, c is 2, 3, or 4. In some embodiments, c is 3. In some embodiments, d is 1, 2, or 3. In some embodiments, d is 1. In some embodiments, e is 1 or 2. In some embodiments, e is 1. In other embodiments, e is 2. In some embodiments, f is 0 or 1. In some embodiments, f is 0. In other embodiments, f is 1.

In some embodiments, R₂₁ is NH₂. In other embodiments, R₂₁ is N₃. In some embodiments, R₂₂ is N₃. In some embodiments, R₂₃ is N₃. In other embodiments, R₂₃ is hydrogen. In other embodiments, R₂₃ is heteroaryl_{(C≤12)} or substituted heteroaryl_{(C≤12)}. In some embodiments, R₂₃ is heteroaryl_{(C≤12)} such as tetrazine or 3-methyltetrazine. In other embodiments, R₂₃ is -arenediyl_{(C≤12)}-heteroaryl_{(C≤12)} or substituted -arenediyl_{(C≤12)}-heteroaryl_{(C≤12)}. In some embodiments, R₂₃ is -arenediyl_{(C≤12)}-heteroaryl_{(C≤12)}. In some embodiments, R₂₃ is 4-tetrazyl-phenyl or 4-(3-methyltetrazyl)-phenyl.

In some embodiments, the polyvalent linker is further defined as:

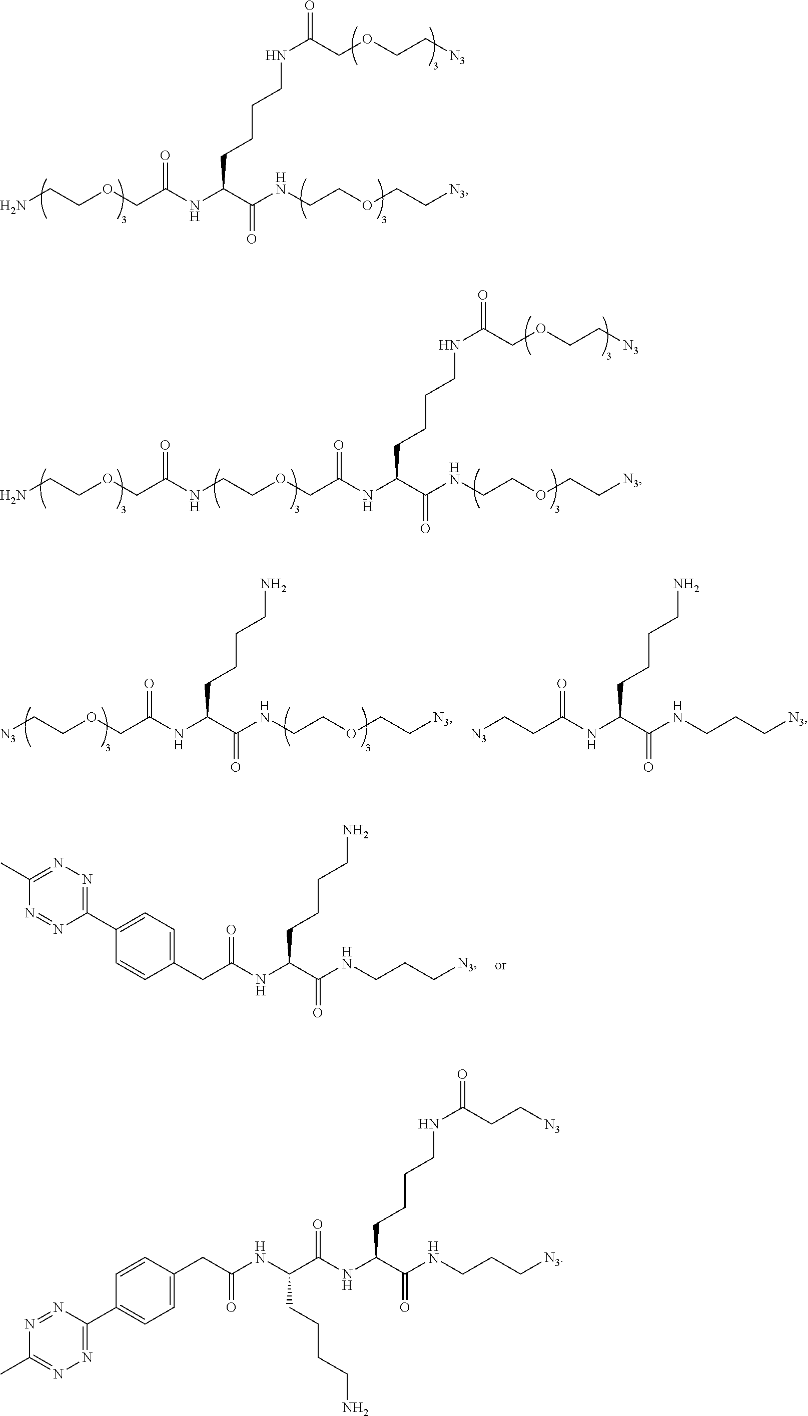

In some embodiments, the compound comprises R₁₆ as a chemotherapeutic drug. In some embodiments, the chemotherapeutic drug is auristatin E (AE), auristatin F (AF), monomethyl auristatin E (MMAE), monomethyl auristatin F (MMAF), dolastatine, maytansine, duocarmycin, tubulysin, chalicheamicin, pyrrobenzodiazepine dimer, anthracycline, paclitaxel, vinblastine, amanitin, eribulin, or a derivative of either of the molecules. In some embodiments, the chemotherapeutic drug is MMAE or MMAF. In some embodiments, the antibody whose antigen is a tumor associated antigen. In some embodiments, the linker is linked to a second antibody or a second compound. In some embodiments, the second antibody is different from the antibody. In some embodiments, the second compound is different from the compound.

In still yet another aspect, the present disclosure provides pharmaceutical compositions comprising a compound or drug conjugate described and an excipient. In some embodiments, the compound is formulated for oral, intraadiposal, intraarterial, intraarticular, intracranial, intradermal, intralesional, intramuscular, intranas al, intraocular, intrapericarial, intraperitoneal, intrapleural, intraprostatical, intrarectal, intrathecal, intratracheal, intratumoral, intraumbilical, intravaginal, intravenous, intraventricular, intravesicularal, intravitreal, liposomal, local, mucosal, parenteral, rectal, subconjunctival, subcutaneous, sublingual, topical, transbuccal, transdermal, vaginal, via a catheter, via a lavage, via continuous infusion, via infusion, via inhalation, via injection, via local delivery, or via localized perfusion administration.

In still another aspect, the present disclosure provides methods of treating a disease or disorder in a patient comprising administering to the patient a therapeutically effective amount of a compound, drug conjugate, or pharmaceutical composition described herein. In some embodiments, the disease or disorder is cancer. In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In other embodiments, the disease or disorder is a microbial infection. In other embodiments, the disease or disorder is an autoimmune disease. In other embodiments, the disease or disorder is associated with inflammation. In other embodiments, the disease or disorder is diabetes. In other embodiments, the disease or disorder is a neurological disorder.

In some embodiments, the methods further comprise administering a second therapy. In some embodiments, the patient is a mammal. In some embodiments, the patient is a human. In other embodiments, the patient is a mouse, rat, dog, or monkey. In some embodiments, the compound, drug conjugate, or composition is administered once. In other embodiments, the compound, drug conjugate, or composition is administered two or more times.

In still yet another aspect, the present disclosure provides uses of the present compounds, drug conjugates, or pharmaceutical compositions in the preparation of a medicament for the treatment of a disease or disorder. In some aspects, the present disclosure may also provide compositions, compounds, or drug conjugates for use in the preparation of a medicament.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a method, composition, kit, or system that “comprises,” “has,” “contains,” or “includes” one or more recited steps or elements possesses those recited steps or elements, but is not limited to possessing only those steps or elements; it may possess (i.e., cover) elements or steps that are not recited. Likewise, an element of a method, composition, kit, or system that “comprises,” “has,” “contains,” or “includes” one or more recited features possesses those features, but is not limited to possessing only those features; it may possess features that are not recited.

Any embodiment of any of the present methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description.

FIGS. 1A-1C: Structures and stability profiles of cleavable peptide linkers. (FIG. 1A) VCit-based ADC linker. VCit linkers are unstable in mouse circulation due to susceptibility to the extracellular carboxylesterase Ces1c. VCit linkers are also labile to human neutrophil elastase-mediated degradation. This instability often triggers premature payload release, leading to poor efficacy in preclinical rodent models and safety concerns including neutropenia and liver toxicity in humans. (FIG. 1B) EVCit-based ADC linker. EVCit linkers developed previously are stable in human and mouse plasma. However, this linker is not capable of withstanding neutrophil elastase-mediated degradation, raising the same safety concerns as is the case with VCit linkers. (FIG. 1C) EGCit-based ADC linker. This study demonstrates that EGCit linkers resist degradation in circulation and cleavage mediated by human neutrophil proteases, while capable of releasing payloads in a traceless manner upon intracellular cleavage.

FIGS. 2A-2E: Incorporation of glycine at P2 and glutamic acid at P3 affords high resistance to undesired degradation. (FIG. 2A) ESI-MS-based peptide mapping of the cleavage site in the presence of human neutrophil elastase. Cleavage of the amide bond between valine and citrulline was observed within VCit (1) and EVCit (2) linkers. (FIG. 2B) Structures of small-molecule P2 probes containing EGCit (3a), EACit (3b), ELCit (3c), EICit (3d), EV(Me)Cit (3e), or GCit (3f). A pyrene group was used as a surrogate of hydrophobic ADC payloads. (FIGS. 2C-2E) Stability of probes 1, 2, and 3a-f in the presence of human neutrophil elastase (FIG. 2C), in undiluted BALB/c mouse plasma (FIG. 2F), or human plasma (FIG. 2E) at 37° C. (1) light purple diamond; (2) green triangle; (3a) magenta square; (3b) black open circle; (3c) cyan asterisk; (3d) orange open triangle; (3e) purple inversed triangle; (3f) light green open diamond. All assays were performed at least three times in technical duplicate, and representative data from the replicates are shown (n=2). Data are presented as mean values±SEM. PABC, p-aminobenzyloxycarbonyl.

FIGS. 3A-3J: EGCit linker increases ADC hydrophilicity and cell killing potency with efficient intracellular payload release. (FIG. 3A) Construction of ADCs (4a-e) by MTGase-mediated branched linker conjugation and following strain-promoted azide-alkyne cycloaddition (yellow spark: MMAE). (FIG. 3B) Deconvoluted ESI-mass spectrum of EGCit ADC 4c. Asterisk (*) indicates a fragment ion detected in ESI-MS analysis. (FIG. 3C) Overlay of five HIC traces (VCit ADC 4a: light purple; EVCit ADC 4b: green; EGCit ADC 4c: magenta; EV(N-Me)Cit ADC 4d: purple; GCit ADC 4e: light green) under physiological conditions (phosphate buffer, pH 7.4). (FIGS. 3D-3I) Cell killing potency in the breast cancer cell lines KPL-4 (FIG. 3D), SK-BR-3 (FIG. 3E), BT-474 (FIG. 3F), JIMT-1 (FIG. 3G), MDA-MB-453 (FIG. 3H), and MDA-MB-231 (FIG. 3I). Unconjugated N297A anti-HER2 mAb (black circle), VCit ADC 4a (light purple diamond), EVCit ADC 4b (green triangle), EGCit ADC 4c (magenta square), EV(N-Me)Cit 4d (purple inversed triangle), GCit ADC 4e (light green open diamond), non-cleavable ADC 4f (cyan open circle), and nontargeting EGCit ADC 5 (isotype control, black open rectangle with dotted curve) were tested. (FIG. 3J) ESI-MS-based quantification of free MMAE released from ADCs 4a-c in KPL-4 cells after incubation at 37° C. for 24 h. All assays were performed in quadruplicate (FIGS. 3D-3I) or triplicate (FIG. 3J). Data are presented as mean values±SEM. For statistical analysis, a one-way ANOVA with a Dunnett's post hoc test was used (comparison control: EGCit ADC 4c). BCN, bicyclo[6.1.0]nonyne; DAR, drug-to-antibody ratio; MMAE, monomethyl auristatin E; MTGase, microbial transglutaminase; PEG, polyethylene glycol.

FIGS. 4A-4I: EGCit ADC is stable in plasma and spares human differentiating neutrophils derived from the bone marrow. (FIGS. 4A-4C) Stability in undiluted mouse plasma (FIG. 4A), cynomolgus monkey plasma (FIG. 4B), and human plasma at 37° C. VCit ADC 4a (light purple diamond), EVCit ADC 4b (green triangle), and EGCit ADC 4c (magenta square) were tested. (FIG. 4D) ESI-MS traces of ADCs 4b, c after incubation with human neutrophil elastase at 37° C. for 24 h. EGCit ADC 4c underwent no cleavage, whereas VCit ADC 4b underwent linker degradation and lost a part of payloads. (FIG. 4E) Study schedule for differentiation of human bone marrow HSPCs into neutrophils and following treatment with ADCs 4b, c. After 3-day expansion (Day 0), HSPCs were treated with growth factors for 7 days and differentiated into CD15+ and CD66b+ granulocytes/neutrophils. (FIGS. 4F-4G) Flow cytometry before (Day 0, FIG. 4F) and after (Day 7, FIG. 4G) differentiation. (FIGS. 4H-4I) Effect of ADCs (vehicle, dark gray; VCit ADC 4a, light purple; EGCit ADC 4c, magenta) on the population of human neutrophils (FIG. 4H) and the viability of all hematopoietic cells (FIG. 4I) relative to those of vehicle control (n=3). All assays were performed in triplicate. Data are presented as mean values±SEM. For statistical analysis, a one-way ANOVA with a Dunnett's post hoc test was used (comparison control: EGCit ADC 4c). HSPCs, hematopoietic stem and progenitor cells.

FIGS. 5A-5D: EGCit linker has the potential to minimize antigen-independent liver toxicity of ADCs. (FIGS. 5D-5D) Blood chemistry parameters (ALT (FIG. 5A), AST (FIG. 5B), ALKP (FIG. 5C), and BUN (FIG. 5D)) measured 5 days post ADC injection to female CD-1® mice. Mice were injected with a single dose of vehicle control (n=4), EGCit ADC 4c (magenta square, n=4), ENHERTU® (purple inversed triangle, n=3), or KADCYLA® (light purple square, n=3) at 80 mg kg⁻¹. Data are presented as mean values (bars)±SEM. For statistical analysis, a Welch's t-test (two-tailed, unpaired, uneven variance) was used. To control the family-wise error rate in multiple comparisons, crude P values were adjusted by the Holm-Bonferroni method. ALKP, alkaline phosphatase; ALT, alanine transaminase; AST, aspartate transaminase; BUN, blood urea nitrogen.

FIGS. 6A-6F: EGCit ADCs exert improved antitumor effects in various xenograft models compared to conventional ADCs. (FIGS. 6A-6D) Anti-tumor activity (FIGS. 6A, 6C) and survival benefit (FIGS. 6B, 6D) in orthotopic xenograft mouse models of human breast cancer. KPL-4 model (FIGS. 6A-6B): a single dose of vehicle control (black circle), KADCYLA® (light purple square), ENHERTU® (purple inversed triangle), EVCit-MMAE ADC 4b (green triangle), EGCit-MMAE ADC 4c (magenta square) or EGCit-DuoDM ADC 6 (cyan circle) was intravenously administered at 1 mg kg⁻¹ to tumor-bearing female NSG mice at a mean tumor volume of ˜100 mm³ (n=5). JIMT-1/MDA-MB-231 4:1 admixed model (FIGS. 6C-6D): eight days post implantation (indicated with a black arrow), female NU/J mice were intravenously administered with a single dose of ENHERTU® (3 mg kg⁻¹, purple inversed triangle, n=5) or EGCit-MMAE/F DAR 4+2 dual-drug ADC 7a, (1 mg kg⁻¹, magenta open square, n=6). Note: The tumor volume and survival curve data of vehicle control (black circle with dotted curve, n=4) and EVCit dual-drug ADC 7b (1 mg kg⁻¹, green open triangle with dotted curve, n=5) presented here have been previously generated. Data are presented as mean values (bars)±SEM. (FIGS. 6E-6F) Study schedule in the U87ΔEGFR-luc orthotopic xenograft model (FIG. 6E) and survival curves after treatment (FIG. 6F). U87ΔEGFR-luc cells were intracranially implanted to male and female NSG mice. Five days post implantation, mice were intravenously administered with a single dose of vehicle control, anti-EGFRvIII VCit-MMAE ADC (8a, 5 mg kg⁻¹, light purple), or anti-EGFRvIII EGCit-MMAE ADC (8b, 5 mg kg⁻¹, magenta). n=6 for vehicle and 8a; n=7 for 8b. All animals other than the ones that were found dead or achieved complete remission were killed at the pre-defined humane endpoint, which were counted as deaths. For statistical analysis of the tumor volume data, a Welch's t-test (two-tailed, unpaired, uneven variance) was used. Kaplan-Meier survival curve statistics were analyzed with a logrank (Mantel-Cox) test. To control the family-wise error rate in multiple comparisons, crude P values were adjusted by the Holm-Bonferroni method. DuoDM, duocarmycin DM.

FIGS. 7A-7B: Stability of probes S1a-d in the presence of human neutrophil elastase (FIG. 7A) or in undiluted BALB/c mouse plasma (FIG. 7B) at 37° C. (S1a) green open triangle; (S1b) cyan open square; (S1c) purple asterisk; (S1d) black cross. All assays were performed in duplicate. Data are presented as mean values±SEM.

FIGS. 8A-8C: SEC analysis of ADCs 4a, c, d before and after 28-day incubation at 37° C. in PBS (pH 7.4). VCit ADC 4a (FIG. 8A), EGCit ADC 4c (FIG. 8B), and EV(N-Me)Cit ADC 4d (FIG. 8C).

FIGS. 9A-9D: Gating strategy and CD15/CD66b expression in differentiated hematopoietic cells. (FIG. 9A) Gating strategy. Scatter, single cell selection, and CD15/CD66b-negative areas were set up using unstained cells from the vehicle control group. ULTRACOMP EBEADS™ Compensation Beads (Invitrogen) labeled with CD15-APC and/or CD66b-FITC were also used to set voltages and gating parameters for obtaining accurate fluorescence signal. Isotype control IgG-APC and -FITC treated cells were used to confirm the specificity of the CD15-APC and CD66b-FITC antibodies. The same gating strategy was used for flow cytometry presented in (FIGS. 9B-9D) in this figure. (FIGS. 9B-9D) Representative 2D-histograms of differentiated hematopoietic cells after a 7-day treatment with vehicle control (FIG. 9B), 200 nM EVCit-MMAE ADC 4b (FIG. 9C), and 200 nM EGCit-MMAE ADC 4c (FIG. 9D). All experiments were performed in triplicate. Data were acquired using a LSR II flow cytometer (BD Biosciences) and Diva acquisition software (version 8.0.1, BD Biosciences) and analyzed using FlowJo analysis software (v10.8.1, FlowJo, LLC).

FIGS. 10A-10D: Tolerability and hematology analysis. (FIG. 10A) Body weight change after female CD-1® mice were administered with a single dose (80 mg kg⁻¹) of vehicle control (n=4), KADCYLA® (n=6), ENHERTU® (n=7), or EGCit ADC 4c (n=7). No mice showed acute symptoms or reached the pre-defined humane endpoint during 5-day monitoring. (FIGS. 10B-10D) Counts of red blood cells (RBC, FIG. 10B), platelets (PLT, FIG. 10C), and neutrophils (NEUT, FIG. 10D) 5 days post injection of a single dose (80 mg kg⁻¹) of vehicle control (n=4), EGCit ADC 4c (n=4), ENHERTU® (n=3), or KADCYLA® (n=3). Dotted lines (High and Low) represent 95% confidential interval of each parameter in healthy CD-1® mice (data from Charles River Laboratories). Data are presented as mean values (bars)±SEM.

FIGS. 11A-11D: Additional in vitro cytotoxicity data. (FIGS. 11A-11B) Cytotoxicity of anti-HER2 EGCit-DuoDM ADC (DAR 4, 6, cyan circle) and anti-HER2 EGCit-MMAE/F dual-drug ADC (DAR 4+2, 7a, magenta open square) in KPL-4 (FIG. 11A) and JIMT-1 (FIG. 11B). (FIG. 11C) Cytotoxicity of anti-EGFRvIII VCit-MMAE ADC (DAR 4, 8a, light purple triangle) and anti-EGFRvIII EGCit-MMAE ADC (DAR 4, 8b, magenta square) in U87ΔEGFR-luc. (FIG. 11D) Cytotoxicity of anti-EGFRvIII EGCit-PABC-DuoDM ADC (DAR 4, S21a, orange square) and anti-EGFRvIII EGCit-PABQ-DuoDM ADC (DAR 4, S21b, purple diamond) in U87ΔEGFR-luc. Concentrations (nM) are based on the antibody dose without normalizing to each DAR. All assays were performed in quadruplicate. Data are presented as mean values+/−SEM (n=4 for 6 and 7a; n=3 for 8a, b and S21a, b).

FIGS. 12A-12E: Body weight change and antitumor activity in various xenograft models. (FIGS. 12A-12B) Body weight change (FIG. 12A) and tumor volume change (FIG. 12B) during the treatment of KPL-4 tumor-bearing mice with each ADC at 1 mg kg⁻¹. Mice were injected intravenously with a single dose of vehicle control (black circle), KADCYLA® (light purple square), ENHERTU® (purple inversed triangle), EVCit-MMAE ADC 4b (green triangle), EGCit-MMAE ADC 4c (magenta square) or EGCit-DuoDM ADC 6 (cyan circle) at a tumor volume of ˜100 mm³. (FIGS. 12C-12D) Body weight change (FIG. 12C) and tumor volume change (FIG. 12D) during the treatment of JIMT-1/MDA-MB-231 admixed tumor-bearing mice. Mice were injected intravenously with a single dose of ENHERTU® (3 mg kg⁻¹, purple inversed triangle, n=5) or EGCit-MMAE/F DAR 4+2 dual-drug ADC 7a, (1 mg kg⁻¹, magenta open square, n=6). Note: The tumor volume and survival curve data of vehicle control (black circle with dotted curve, n=4) and EVCit dual-drug ADC 7b (1 mg kg⁻¹, green open triangle with dotted curve, n=5) presented here have been previously generated. e Body weight change during the treatment of orthotopic U87ΔEGFR-luc tumor-bearing mice with each ADC at 5 mg kg⁻¹. Mice were intravenously administered with a single dose of vehicle control, anti-EGFRvIII VCit-MMAE ADC 8a (light purple), or anti-EGFRvIII EGCit-MMAE ADC 8b (magenta). n=6 for vehicle and 8a; n=7 for 8b. Data are presented as mean values+/−SEM.

FIG. 13: Synthesis of pyrene probes. Reagents and conditions: (a) Fmoc-citrulline, DIPEA, DMF, room temp, 2 h; (b) p-aminobenzyl alcohol, EEDQ, DCM/MeOH=4:1, room temp, overnight; (c) bis(4-nitrophenyl) carbonate, DMAP, DMF, room temp, 2 h; (d) 20% TFA/DCM, room temp, 40 min for S3a or 1N-HCl/ACN, room temp, 1-3 h for S3b-e and S3g, h; (e) sarcosine-pyrene, DMAP, DIPEA, DMF, 37° C., 4 h.

FIG. 14: Synthesis of BCN-MMAE modules (a-c). Reagents and conditions: (a) p-aminobenzyl alcohol, EEDQ, DCM/MeOH=4:1, room temp, overnight; (b) bis(4-nitrophenyl) carbonate, DMAP, DMF, room temp, 2 h; (c) 20% TFA/DCM, room temp, 40 min for S5a and S5b; (d) MMAE, HOAt, DIPEA, DMF, 37° C., overnight; (e) 50% diethylamine/DMF, room temp, 1 h; (f) BCN-NHS, DIPEA, DMF, room temp, 3 h.

FIG. 15: Synthesis of BCN-EVCit-MMAE module S8. Reagents and conditions: (a) 20% TFA/DCM, room temp, 1 h; (b) 50% diethylamine/DMF, room temp, 1 h; (c) BCN-NHS, DIPEA, DMF, room temp, overnight.

FIG. 16: Synthesis of non-cleavable-MMAE module S10. Reagents and conditions: (a) Boc-peg4-acid, HATU, DIPEA, DMF, room temp, 1 h; (b) 50% TFA/DCM, room temp, 30 min; (c) BCN-NHS, DIPEA, DMF, room temp, 30 min.

FIG. 17: Synthesis of TCO-EGCit-MMAF module S12. Reagents and conditions: (a) 20% TFA/DCM, room temp, 50 min; (b) MMAF, HOAt, DIPEA, DMF, 37° C., overnight; (c) 50% diethylamine/DMF, room temp, 30 min; (d) TCO-NHS, DIPEA, DMF, room temp, 3.5 h.

FIG. 18: Synthesis of BCN-DuoDM module (S15). Reagents and conditions: (a) 4-nitrophenyl chloroformate, DIPEA, ACN, room temp, 30 min; (b) t-butyl methyl(2-(methylamino)ethyl)carbamate, room temp, 15 min; (c) 20% TFA/DCM, room temp, 50 min for S5a, 50% TFA/DCM, 0° C., 30 min for S13; (d) Boc-deprotected S11, DIPEA, DMF, room temp, 2 h; (e) 50% diethylamine/DMF, room temp, 30 min; (f) BCN-NHS, DIPEA, DMF, room temp, 1 h.

FIG. 19: Synthesis of BCN-DuoDM-glucuronide module (S20). Reagents and conditions: (a) BF₃·Et₂O, methyl-(2,3,4-tri-O-acetyl-α-D-glucopyranosyl trichloroacetimidate, DCM, MS (4 Å), −20° C., 2 h then BF₃·Et₂O, room temp, 2 h; (b) 5-(2-dimethylaminoethoxy)indole-2-carboxylic acid, EDC·HCl, DMF, room temp, 1.5 h; (c) thionyl chloride, DCM, DMF, 0° C., 2.5 h; (d) S17, DIPEA, NaI, DMF, room temp, overnight; (e) 20% TFA/DCM, room temp, 1 h; (f) LiOH·H₂O, MeOH, room temp, 1 h; (g) BCN-NHS, DIPEA, DMF, room temp, 1.5 h.

FIG. 20: HPLC and ESI-MS data for S3a.

FIG. 21: HPLC and ESI-MS data for S3b.

FIG. 22: HPLC and ESI-MS data for S3c.

FIG. 23: HPLC and ESI-MS data for S3d.

FIG. 24: HPLC and ESI-MS data for S3e.

FIG. 25: HPLC and ESI-MS data for S3f.

FIG. 26: HPLC and ESI-MS data for S3g.

FIG. 27: HPLC and ESI-MS data for S3h.

FIG. 28: HPLC and ESI-MS data for S3i.

FIG. 29: HPLC and ESI-MS data for S3j.

FIG. 30: HPLC and ESI-MS data for 3a.

FIG. 31: HPLC and ESI-MS data for 3b.

FIG. 32: HPLC and ESI-MS data for 3c.

FIG. 33: HPLC and ESI-MS data for 3d.

FIG. 34: HPLC and ESI-MS data for 3e.

FIG. 35: HPLC and ESI-MS data for 3f.

FIG. 36: HPLC and ESI-MS data for S1a.

FIG. 37: HPLC and ESI-MS data for S1b.

FIG. 38: HPLC and ESI-MS data for S1c.

FIG. 39: HPLC and ESI-MS data for S1d.

FIG. 40: HPLC and ESI-MS data for S5a.

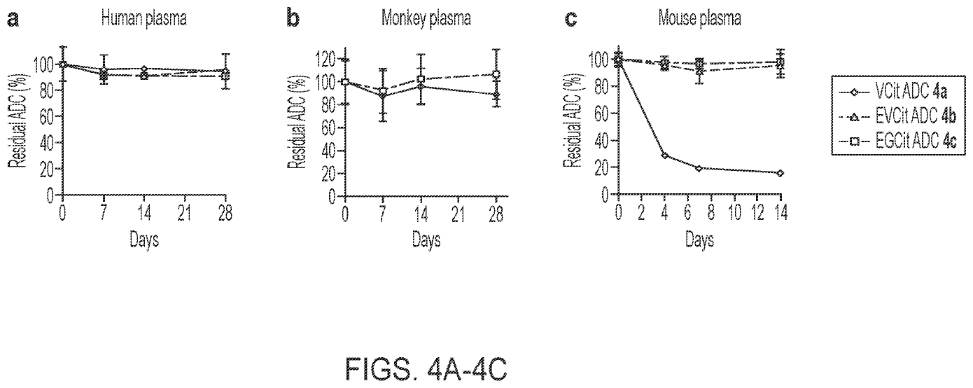

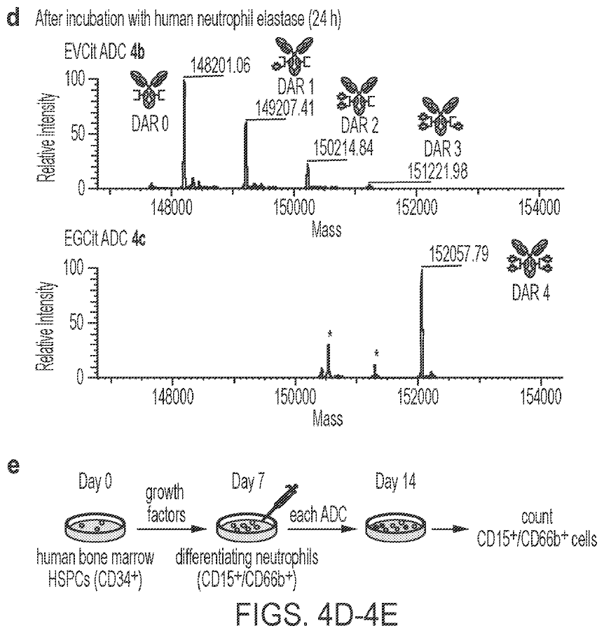

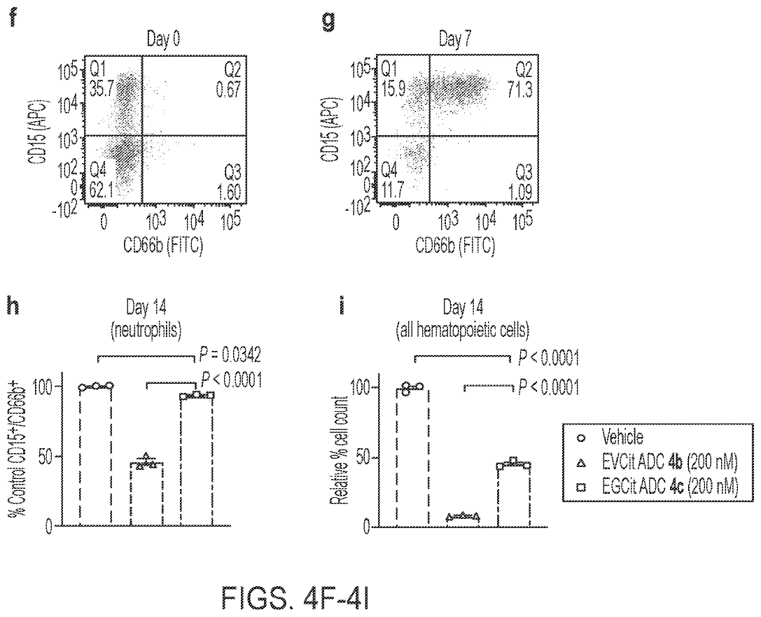

FIG. 4I: HPLC and ESI-MS data for S5b.

FIG. 42: HPLC and ESI-MS data for S5c.

FIG. 43: HPLC and ESI-MS data for S6a.

FIG. 44: HPLC and ESI-MS data for S6b.

FIG. 45: HPLC and ESI-MS data for S6c.

FIG. 46: HPLC and ESI-MS data for S7a.

FIG. 47: HPLC and ESI-MS data for S7b.

FIG. 48: HPLC and ESI-MS data for S7c.

FIG. 49: HPLC and ESI-MS data for S8.

FIG. 50: HPLC and ESI-MS data for S9.

FIG. 51: HPLC and ESI-MS data for S10.

FIG. 52: HPLC and ESI-MS data for S11.

FIG. 53: HPLC and ESI-MS data for S12.

FIG. 54: HPLC and ESI-MS data for S13.

FIG. 55: HPLC and ESI-MS data for S14.

FIG. 56: HPLC and ESI-MS data for S15.

FIG. 57: HPLC and ESI-MS data for S16.

FIG. 58: HPLC and ESI-MS data for S17.

FIG. 59: HPLC and ESI-MS data for S18.

FIG. 60: HPLC and ESI-MS data for S19.

FIG. 61: HPLC and ESI-MS data for S20.

FIGS. 62A-62F: ESI-mass spectra of VCit ADC 4a (FIG. 62A), EVCit ADC 4b (FIG. 62B), EV(N-Me)Cit 4d (FIG. 62C), GCit ADC 4e (FIG. 52D), non-cleavable ADC 4f (FIG. 62E), Isotype control EGCit ADC 5 (FIG. 62F). Asterisk (*) indicates fragment ion(s) detected in ESI-MS analysis.

FIGS. 63A-63B: ESI-mass spectra of anti-HER2 EGCit DuoDM ADC 6 (FIG. 63A) and anti-HER2 EGCit MMAE/F dual-drug ADC 7a (FIG. 63B). Asterisk (*) indicates fragment ion(s) detected in ESI-MS analysis.

FIGS. 64A-64F: ESI-mass spectra of aglycosylated N297A anti-EGFRvIII mAb (FIG. 64A), mAb-linker conjugate (FIG. 64B), anti-EGFRvIII VCit MMAE ADC 8a (FIG. 64C), anti-EGFRvIII EGCit MMAE ADC 8b (FIG. 64D), anti-EGFRvIII EGCit PABC-DuoDM ADC S21a (FIG. 64E), and anti-EGFRvIII EGCit PABQ-DuoDM ADC S21b (FIG. 64F). Asterisk (*) indicates fragment ion(s) detected in ESI-MS analysis.

### DESCRIPTION OF ILLUSTRATIVE EMBODIMENTS

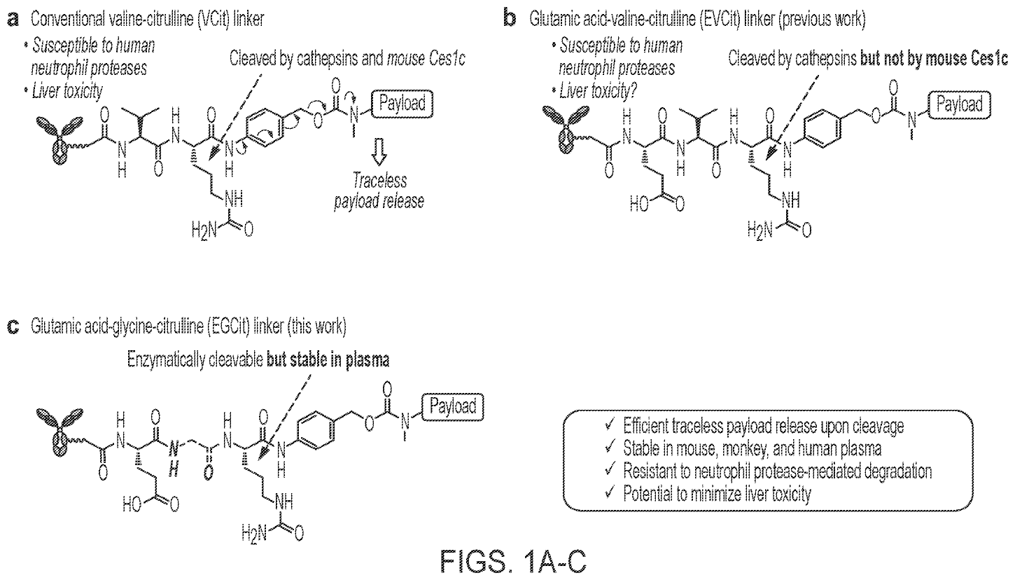

Previous studies demonstrated that a glutamic acid-valine-citrulline (EVCit) tripeptide linker is stable in human and mouse plasma. However, as is the case with VCit linkers and other similar valine-based linkers, the EVCit linker was shown to be susceptible to human neutrophil elastase-mediated degradation, suppressing the population of differentiating neutrophils (FIG. 1B) (WO2018218004; incorporated by reference herein in its entirety). Thus, in certain embodiments, the present disclosure provides glutamic acid-glycine-citrulline (EGCit) tripeptide linkers. The replacement of the valine with a small glycine residue is a unique chemical modification that is contrary to the common belief in the field that a bulky amino acid such as valine is needed to ensure effective payload release upon intracellular cleavage. These linkers can solve or mitigate the clinical issues caused by linker instability (for example, hepatotoxicity, neutropenia, thrombocytopenia, leukopenia, and pancytopenia) without compromising ADC therapeutic efficacy.

Further provided herein are drug conjugates, such as for targeted therapy, comprising the present chemical linkers. The drug conjugates may be antibody drug conjugates. The present chemical linkers contain a glutamic acid-glycine-citrulline (EGCit) tripeptide sequence that provides significantly improved stability in circulation, long-term stability in both rodent and primate plasma, resistance to degradation mediated by neutrophil elastase, and enables traceless drug release upon internalization into the target cell. It was also demonstrated that EGCit-based ADCs targeting HER2 or EGFR showed significantly improved therapeutic efficacy and safety profiles in mouse models of human breast cancer and glioblastoma compared to conventional ADCs, including VCit-based ADCs, KADCYLA® (T-DM1) and ENHERTU® (DS-8201). Notably, the EGCit-based ADC showed no discernable liver toxicity in healthy mice at 80 mg kg⁻¹. These findings showed that the EGCit linker technology not only ensures smooth transition from preclinical studies to in-human evaluation, but also provides a broadly applicable solution for substantially widening therapeutic windows of targeted drug delivery systems including ADCs. The EGCit linker and other linkers with similar chemical compositions can provide a general approach to generating truly effective and safe ADCs and other drug delivery systems for cancer therapy as well as therapies for other diseases. The EGCit linkers do not rely on specific antibody structure or drugs, thus this technology can be used for constructing any type of ADCs. In addition, EGCit linkers are more hydrophilic than VCit linkers, which may reduce the risk of protein aggregation. Thus, the present linkers could lead to novel, efficacious, and safe ADCs for treating cancers and other diseases.

Also provided herein are methods of preparing drug conjugates, such as ADCs, with the present linkers which allow for the incorporation of one or more therapeutic compounds. Using MTGase, antibodies could be coupled with amines comprised of pendant diazide functionality or azide-tetrazine heterobifunctionality. The azide and tetrazine moieties could then be further functionalized through cycloaddition to incorporate chemical payload(s).

### I. DELIVERY OF THERAPEUTIC AGENTS VIA DRUG CONJUGATES

### A. Therapeutic Agents

Any of a number of drugs are suitable for use or can be modified to be rendered suitable for use, as a reactive partner to conjugate to a linker of the present disclosure. Examples of drugs include small molecules, peptide drugs, oligonucleotides, antibodies, and fragments thereof. Thus, the present disclosure provide drug-antibody conjugates.

“Small molecule drug” as used herein refers to a compound, e.g., an organic compound, which exhibits a pharmaceutical activity of interest and which is generally of a molecular weight of 800 Da or less, or 2000 Da or less, but can encompass molecules of up to 5 kDa and can be as large as 10 kDa. A small inorganic molecule refers to a molecule containing no carbon atoms, while a small organic molecule refers to a compound containing at least one carbon atom.

“Peptide drug” as used herein refers to amino-acid containing polymeric compounds, and is meant to encompass naturally-occurring and non-naturally-occurring peptides, oligopeptides, cyclic peptides, polypeptides, and proteins, as well as peptide mimetics. The peptide drugs may be obtained by chemical synthesis or be produced from a genetically encoded source (e.g., recombinant source). Peptide drugs can range in molecular weight, and can be from 200 Da to 10 kDa or greater in molecular weight.

“Oligonucleotides” as used herein refers to therapeutic agents comprising one or more nucleobases such as mRNAs, siRNAs, or other polynucleotide agents that may be used to treat a condition such as a disease or disorder. These oligonucleotides may be naturally occurring or prepared synthetically. These oligonucleotide may be siRNA, a miRNA, a tRNA, a pri-miRNA, a messenger RNA (mRNA), a cluster regularly interspaced short palindromic repeats (CRISPR) related nucleic acid, a single guide RNA (sgRNA), a CRISPR-RNA (crRNA), a trans-activating crRNA (tracrRNA), a plasmid DNA (pDNA), a transfer RNA (tRNA), an antisense oligonucleotide (ASO), a guide RNA, a double stranded DNA (dsDNA), a single stranded DNA (ssDNA), a single stranded RNA (ssRNA), and a double stranded RNA (dsRNA). These nucleic acids may be chemically modified to reduce its ability to be degraded in vivo.

It is contemplated that drug conjugates, such as ADCs, constructed from the compounds of the present disclosure can be used to deliver one or more drugs. In some embodiments, the drug is a cancer chemotherapeutic agent. For example, where die polypeptide is an antibody (or fragment thereof) that has specificity for a tumor cell, the antibody can be modified as described herein to include a modified amino acid, which can be subsequently conjugated to a cancer chemotherapeutic agent. Cancer chemotherapeutic agents include non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Suitable cancer chemotherapeutic agents include dolastatin and active analogs and derivatives thereof; and auristatin and active analogs and derivatives thereof. See, e.g., WO 96/33212, WO 96/14856, and U.S. Pat. No. 6,323,315. For example, dolastatin 10 or auristatin PE can be included in an antibody-drug conjugate of the present disclosure. Suitable cancer chemotherapeutic agents also include maytansinoids and active analogs and derivatives thereof (see, e.g., EP 1,391.213; and Liu et al. 1996); and duocarmycins and active analogs and derivatives thereof (e.g., including the synthetic analogues, KW-2189 and CBI-TMI).

Agents that act to reduce cellular proliferation are known in the art and widely used. Such agents include alkylating agents, such as nitrogen mustards, nitosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine, (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, lO-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, 1-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafme, cyclophosphamide, ifosamide, and droloxafme.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17a-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore compounds that bind to the estrogen receptor are used to block this activity. Corticosteroids may inhibit T cell proliferation.

Other suitable chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (Z) 1839, 4-(3-chloro-4-fluorophenylamino)-7-methoxy-6-(3-(4-morpholinyl)propoxy)quinazoline); etc. Topoisomerase I inhibitor such as irinotecan, topotecan, SN-38, exatecan (part of Deruxtecan) and the other analogs RNA polymerase inhibitors (II and III) such as α-amanitin.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the an (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in international Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ; (8) colony-stimulating factors; and (9) inhibitors of angiogenesis.

### B. Methods for Modification of Drugs to Contain a Reactive Partner

Drugs to be conjugated to a polypeptide may be modified to incorporate a reactive partner for reaction with the polypeptide. Where the drug is a peptide drug, the reactive moiety (e.g., aminooxy or hydrazide can be positioned at an N-terminal region, the N-terminus, a C-terminal region, the C-terminus, or at a position internal to the peptide. For example, an example of a method involves synthesizing a peptide drug having an aminooxy group. In this example, the peptide is synthesized from a Boc-protected precursor. An amino group of a peptide can react with a compound comprising a carboxylic acid group and oxy-N-Boc group. As an example, the amino group of the peptide reacts with 3-(2,5-dioxopyrrolidin-1-yloxy)propanoic acid. Other variations on the compound comprising a carboxylic acid group and oxy-N-protecting group can include different number of carbons in the alkylene linker and substituents on the alkylene linker. The reaction between the amino group of the peptide and the compound comprising a carboxylic acid group and oxy-N-protecting group occurs through standard peptide coupling chemistry. Examples of peptide coupling reagents that can be used include, but not limited to, DCC (dicyclohexylcarbodiimide), DIC (diisopropylcarbodiimide), di-p-toluoylcarbodiimide, BDP (1-benzotriazole diethylphosphate-1-cyclohexyl-3-(2-morpholinylethyl)carbodiimide), EDC (1-(3-dimethylaminopropyl-3-ethyl-carbodiimide hydrochloride), cyanuric fluoride, cyanuric chloride, TFFH (tetramethyl fluoroformamidinium hexafluorophosphosphate), DPPA (diphenylphosphorazidate), BOP (benzotriazol-1-yloxytris(dimethylamino)phosphonium hexatluorophosphate), HBTU (O-benzotriazol-1-yl-N,N,N′,N′-tetramethyluronium hexafluorophosphate), TBTU (O-benzotriazol-1-yl-N,N,N′,N-tetramethyluronium tetrafluorohorate), TSTr (0-(N-succinimidyl)-N,N,N′,N′-tetramethyluronium tetrafluoroborate), HATU (N-(dimethylamino)-1-H-1-1,2,3-triazolo[4,5,6]-pyridin-1-ylmethylene]-N-methylmethanaminium hexafluorophosphate N-oxide), BOP-C1 (bis(2-oxo-3-oxazolidinyl)phosphinic chloride), PyBOP ((1-H-1,2,3-benzotriazol-1-yloxy)-tris(pyrrolidino)phosphonium tetrafluorophosphine), BrOP (bromotris(dimethylamino)phosphonium hexafluorophosphate), DEPBT (3-(diethoxyphosphoryloxy)-1,2,3-benzotriazin-4(3H)-one) PyBrOP (bromotris(pyrrolidino)phosphonium hexafluorophosphate). As a non-limiting example, HOBt and DIC can be used as peptide coupling reagents.

Deprotection to expose the amino-oxy functionality is performed on the peptide comprising an N-protecting group. Deprotection of the N-oxysuccinimide group, for example, occurs according to standard deprotection conditions for a cyclic amide group. Deprotecting conditions can be found in Greene and Wuts, Protective Groups in Organic Chemistry, 3rd Ed., 1999, John Wiley & Sons, NY and Harrison et al. Certain deprotection conditions include a hydrazine reagent, amino reagent, or sodium borohydride. Deprotection of a Boc protecting group can occur with TFA. Other reagents for deprotection include, but are not limited to, hydrazine, methylhydrazine, phenylhydrazine, sodium borohydride, and methylamine. The product and intermediates can be purified by conventional means, such as HPLC purification.

The ordinarily skilled artisan will appreciate that factors such as pH and steric hindrance (i.e., the accessibility of the amino acid residue to reaction with a reactive partner of interest) are of importance. Modifying reaction conditions to provide for optimal conjugation conditions is well within the skill of the ordinary artisan, and is routine in the art. Where conjugation is conducted with a polypeptide present in or on a living cell, the conditions are selected so as to be physiologically compatible. For example, the pH can be dropped temporarily for a time sufficient to allow for the reaction to occur but within a period tolerated by the cell (e.g., from about 30 min to 1 hour). Physiological conditions for conducting modification of polypeptides on a cell surface can be similar to those used in a ketone-azide reaction in modification of cells bearing cell-surface azides (see, e.g., U.S. Pat. No. 6,570,040).

Small molecule compounds containing, or modified to contain, a nucleophilic group that serves as a reactive partner with a compound or conjugate disclosed herein are also contemplated for use as drugs in the polypeptide-drug conjugates of the present disclosure. General methods are known in the art for chemical synthetic schemes and conditions useful for synthesizing a compound of interest (see, e.g., Smith and March, March's Advanced Organic Chemistry: Reactions, Mechanisms, and Structure, Fifth Edition, Wiley-Interscience, 2001; or Vogel. A Textbook of Practical Organic Chemistry, Including Qualitative Organic Analysis, Fourth Edition, New York: Longman, 1978).

### C. Peptide Drugs

In some cases, a conjugate comprises a covalently linked peptide. Suitable peptides include, but are not limited to, cytotoxic peptides; angiogenic peptides; anti-angiogenic peptides; peptides that activate B cells; peptides that activate T cells; anti-viral peptides; peptides that inhibit viral fusion; peptides that increase production of one or more lymphocyte populations; anti-microbial peptides; growth factors; growth hormone-releasing factors; vasoactive peptides; anti-inflammatory peptides; peptides that regulate glucose metabolism; an anti-thrombotic peptide; an anti-nociceptive peptide; a vasodilator peptide; a platelet aggregation inhibitor; an analgesic; and the like.

In some embodiments, the peptide can be chemically synthesized to include a group reactive with an amino acid residue or a modified amino acid residue of the polypeptide. A suitable synthetic peptide has a length of from 5 amino acids to 100 amino acids, or longer than 100 amino acids: e.g., a suitable peptide has a length of from 5 amino acids (aa) to 10 aa, from 10 aa to 15 aa, from 15 aa to 20 aa, from 20 aa to 25 aa, from 25 aa to 30 aa, from 30 aa to 40 aa, from 40 aa to 50 aa, from 50 aa to 60 aa, from 60 aa to 70 aa, from 70 an to 80 aa, from 80 aa to 90 aa, or from 90 aa to 100 aa.

In certain embodiments, a peptide can be modified to contain a nucleophile-containing moiety (e.g., an aminooxy or hydrazide moiety), e.g., can be reacted with an fGly-containing polypeptide to yield a conjugate in which the polypeptide and peptide are linked by a hydrazone or oxime bond, respectively. Examples of methods of synthesizing a peptide, such that the synthetic peptide comprising a reactive group reactive with an amino acid residue or a modified amino acid residue of the polypeptide, are described above.

Suitable peptides include, but are not limited to, hLF-11 (an 11-amino acid N-terminal fragment of lactoferrin), an anti-microbial peptide; granulysin, an anti-microbial peptide; Plectasin (NZ21 14; SAR 215500), an anti-microbial peptide; viral fusion inhibitors such as Fuzeon (enfuvirtide), TRI-1249 (T-1249; see, e.g., Matos et al., 2010), TRI-2635 (T-2635; see, e.g., Eggink et al., 2009), T651, and TRI-1144; C5a receptor inhibitors such as PMX-53, JPE-1375, and JSM-7717; POT-4, a human complement factor C3 inhibitor, Pancreate (an LNGAP derivative sequence, a HIP-human proislet protein); somatostatin; a somatostatin analog such as DEBIO 8609 (Sanvar), octreotide, octreotide (C2L), octreotide QLT, octreotide LAR, Sandostatin LAR, SomaLAR, Somatuline (lanreotide), see, e.g., Deghenghi et al., 2001; T-19507 (Tesamorelin, a growth hormone-releasing factor); POL7080 (a protegrin analog, an anti-microbial peptide); relaxin; a corticotropin releasing factor agonist such as urotensin, sauvagine, and the like; a heat shock protein derivative such as DiaPep277; a human immunodeficiency virus entry inhibitor, a heat shock protein-20 mimic such as AZX100; a thrombin receptor activating peptide such as TP508 (Chrysalin); a urocortin 2 mimic (e.g., a CRF2 agonist) such as urocortin-2; an immune activator such as Zadaxin (thymalfasin; thymosin-al), see, e.g., Sjogren (2004) J. Gastroenterol. Hepatol. 19:569; a hepatitis C virus (HCV) entry inhibitor E2 peptide such as HCV3; an atrial natriuretic peptide such as HANP (Sun 4936; carperitide); an annexin peptide; a defensin (anti-microbial peptide) such as hBD2-4; a defensin (anti-microbial peptide) such as hBD-3; a defensin (antimicrobial peptide) such as PMX-30063; a histatin (anti-microbial peptide) such as histatin-3, histatin-5, histatin-6, and histatin-9; a histatin (anti-microbial peptide) such as PAC-113; an indolicidin (anti-microbial peptide) such as MX-594AN (Omniganin; CLSOOl); an indolicidin (anti-microbial peptide) such as Omnigard (MBI-226; CPI-226); an anti-microbial peptide such as an insect cecropin; an anti-microbial peptide such as a lactoferrin (talactoferrin); an LL-37/cathelicidin derivative (an anti-microbial peptide) such as P60.4 (OP-145); a magainin (an anti-microbial peptide) such as Pexiganan (MSI-78; Suponex); a protegrin (an anti-microbial peptide) such as IB-367 (Iseganan); an agan peptide; a beta-natriuretic peptide such as Natrecor, or Noratak (Nesiritide), or ularitide; bivalarudin (Angiomax), a thrombin inhibitor; a C peptide derivative; a calcitonin such as Miacalcin (Fortical); an enkephalin derivative; an erythropoiesis-stimulating peptide such as Hematide; a gap junction modulator such as Danegaptide (ZP1609); a gastrin-releasing peptide; a ghrelin; a glucagon-like peptide; a glucagon-like peptide-2 analog such as ZP1846 or ZP1848; a glucosaminyl muramyl dipeptide such as GMDP; a glycopeptide antibiotic such as Oritavancin; a teicoplanin derivative such as Dalbavancin; a gonadotropin releasing hormone (GnRH) such as Zoladex (Lupon) or Triptorelin; a histone deacetylase (HDAC) inhibitor depsipeptide such as PM02734 (Irvalec); an integrin such as eptifibatide; an insulin analog such as Humulog; a kahalalide depsipeptide such as PM02734; a kallikrein inhibitor such as Kalbitor (ecallantide); an antibiotic such as Telavancin; a lipopeptide such as Cubicin or MX-2401; a lutenizing hormone releasing hormone (LHR-H) such as goserelin; an LHRH synthetic decapeptide agonist analog such as Treistar (triptorelin pamoate); an LHRH such as Eligard; an M2 protein channel peptide inhibitor; metreleptin; a melanocortin receptor agonist peptide such as bremalanotide/PT-141; a melanocortin; a muramyl tripeptide such as Mepact (mifamunide); a myelin basic protein peptide such as MBP 8298 (dinucotide); an N-type voltage-gated calcium channel blocker such as Ziconotide (Prialt); a parathyroid hormone peptide; a parathyroid analog such as 768974; a peptide hormone analog such as UGP281; a prostaglandin F2-a receptor inhibitor such as PDC31; a protease inhibitor such as PPL-100; surfaxin; a thromobspondin-1 (TSP-1) mimetic such as CVX-045 or ABT 510; a vasoactive intestinal peptide; vasopressin; a Y2R agonist peptide such as RG7089; obinepeptide; and TM30339.

### II. LINKERS

In some aspects, the present disclosure provides linkers which may be used to connect one or more different drugs and/or cell targeting moieties to a drug molecule.

In other aspects, the linker may further comprise one or more spacer groups. The spacer groups may comprise an amino acid sequence which is recognized by a peptidase in vivo and result in the cleavage of the cell targeting moiety from the drug. In some embodiments, the peptidase is an endosomal or lysosomal peptidase. In other embodiments, the peptidase is an extracellular peptidase such as a matrix metalloproteases, thimet oligopeptidase, or CD10. One example is the amino acid sequence valine-citrulline which is cleavable by cathepsin B. Other non-limiting examples of peptide sequences that are cleavable include valine-alanine, valine-lysine, valine-omithine, phenylalanine-alanine, phenylalanine-lysine, and phenylalanine-ornithine. Additionally, it is contemplated that other functional moieties may be added into the spacer group which can be used to achieve decoupling of the drug and the cell targeting moieties including but not limited to hydrazones, disulfide bonds, or esters. It is also contemplated that the space group may further comprise one or more self-immolating groups. As used herein, a self-immolating group is a group which undergoes decomposition once cleaved at one functional group. Self-immolating groups are well known with the context of ADC's and are taught by Carl et al., 1981; WO 81/01145; Dubowchik et al., 1999; U.S. Pat. No. 6,214,345; Told et al., 2002; Doronina et al., 2003 (erratum, p. 941); U.S. Pat. No. 7,691,962; US 2008/0279868; WO 2008/083312; U.S. Pat. No. 7,375,078 B2; US 2003/0096743; Burke et al., 2017; Staben et al., 2016, the entirety of which are incorporated by reference. One particular example of a self-immolating group is para-aminobenzyl alcohol, para-aminobenzyloxycarbonyl, or para-aminobenzyldialykylammonium.

### III. COMPOUNDS AND FORMULATIONS THEREOF

### A. Compounds

The compounds provided by the present disclosure are shown, for example, above in the summary section and in the examples and claims below. They may be made using the methods outlined in the Examples section. The ADCs described herein can be synthesized according to the methods described, for example, in the Examples section below. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

The ADCs described herein may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration.

Chemical formulas used to represent the ADCs described herein will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

The ADCs described herein may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the ADCs described herein are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The ADCs herein may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the ADCs described herein may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

Those skilled in the art of organic chemistry will appreciate that many organic compounds can form complexes with solvents in which they are reacted or from which they are precipitated or crystallized. These complexes are known as “solvates.” For example, a complex with water is known as a “hydrate.” Solvates of the ADCs described herein are within the scope of the disclosure. It will also be appreciated by those skilled in organic chemistry that many organic compounds can exist in more than one crystalline form. For example, crystalline form may vary from solvate to solvate. Thus, all crystalline forms of the ADCs or precursors described herein are within the scope of the present disclosure.

### B. Formulations

In some embodiments of the present disclosure, the drug conjugates, such as ADCs, are included in a pharmaceutical formulation. Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., the ADCs herein) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinyl-pyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

### IV. INDICATIONS

### A. Hyperproliferative Diseases

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, the ADCs described herein may be used to lead to decreased cell counts and as such can potentially be used to treat a variety of types of cancer lines. In some aspects, it is anticipated that the ADCs described herein may be used to treat virtually any malignancy.

Cancer cells that may be treated with the compounds of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### B. Microbial Infection

The compositions of the present disclosure may provide antimicrobial effect to a target microbial organism and can be used to treat a disease or infection associated with the target microbial organism. An antimicrobial effect includes inhibiting the growth or killing of the target microbial organisms, or interfering with any biological functions of the target microbial organisms. In general, the compositions of the present disclosure can be used to treat a disease or infection at any place in a host, e.g., at any tissue including surfaces of any tissue or implant. In one embodiment, the compositions are used to specifically kill or inhibit bacterial target microbial organisms in body fluid (e.g., blood, sputum).

Compositions of the present disclosure may be effective against bacteria including Gram-positive and Gram-negative cocci, Gram-positive and Gram-negative straight, curved and helical/vibroid and branched rods, sheathed bacteria, sulfur-oxidizing bacteria, sulfur or sulfate-reducing bacteria, spirochetes, actinomycetes and related genera, myxobacteria, mycoplasmas, rickettsias and chlamydias, cyanobacteria, archea, fungi, parasites, viruses and algae. For example, the target microbial organisms of the present disclosure include, without limitation, Escherichia coli, Candida, Salmonella, Staphylococcus, and Pseudomonas, Campylobacter jejuni, Candida albicans, Candida krusei, Chlamydia trachomatis, Clostridium difficile, Cryptococcus neoformans, Haempohilus influenzae, Helicobacter pylori, Moraxella catarrhalis, Neisseria gonorrhoeae, Pseudomonas aeroginosa, Salmonella typhimurium, Shigella disenteriae, Staphylococcus aureus, and Streptococcus pneumoniae. In addition, the composition may be used to treat chronic skin ulcers, infected acute wounds or burn wounds, infected skin eczema, impetigo, atopic dermatitis, acne, external otitis, vaginal infections, seborrhoic dermatitis, oral infections, paradontitis, conjunctivitis or pneumonia.

Compositions of the present disclosure may be effective against gram-negative bacteria. Gram-positive and Gram-negative cocci include, but are not limited to, Aerococcus, Enterococcus, Halococcus, Leuconostoc, Micrococcus, Mobiluncus, Moraxella catarrhalis, Neisseria (including N. gonorrheae and N. meningitidis), Pediococcus, Peptostreptococcus, Staphylococcus species (including S. aureus, methicillin-resistant S. aureus, coagulase-negative S. aureus, and S. saprophyticus), Streptococcus species (including S. pyogenes, S. agalactiae, S. bovis, S. pneumoniae, S. mutans, S. sanguis, S. equi, S. equinus, S. thermophlus, S. morbillorum, S. hansenii, S. pleomorphus, and S. parvulus), and Veillonella.

The Gram-positive and Gram-negative straight, curved, helical/vibrioid and branched rods include, but are not limited to, Acetobacter, Acinetobacter, Actinobacillus equuli, Aeromonas, Agrobacterium, Alcaligenes, Aquaspirillum, Arcanobacterium haemolyticum, Bacillus species (including B. cereus and B. anthracis), Bacteroides species (including B. fragilis), Bartonella, Bordetella species (including B. pertussis), Brochothrix, Brucella, Burkholderia cepacia, Calymmatobacterium granulomatis, Campylobacter species (including C. jejuni), Capnocytophaga, Caulobacter, Chromobacterium violaceum, Citrobacter, Clostridium species (including C. perfringens, C. tetani and C. difficile), Comamonas, Curtobacterium, Edwardsiella, Eikenella, Enterobacter, Erwinia, Erysipelothrix, Escherichia species (including E. coli), Flavobacterium species (including E. meninosepticum), Francisella species (including E. tularensis), Fusobacterium (including E. nucleatum), Gardnerella species (including G. vaginalis), Gluconobacter, Haemophilus species (including H. influenzae and H. ducreyi), Hafnia, Helicobacter (including H. pylori), Herpetosiphon, Klebsiella species (including K. pneumoniae), Kluyvera, Lactobacillus, Legionella species (including E. pneumophila), Leptotrichia, Listeria species (including E. monocytogenes), Microbacterium, Morganella, Nitrobacter, Nitrosomonas, Pasteurella species (including P. multocida), Pectinatus, Porphyromonas gingivalis, Proteus species (including E. mirabilis), Providencia, Pseudomonas species (including E. aeruginosa, P. mallei, P. pseudomallei and E. solanacearum), Rahnella, Renibacterium salmoninarum, Salmonella, Serratia, Shigella, Spirillum, Streptobacillus species (including S. monilformis), Vibrio species (including V. cholerae and V. vulnificus), Wolinella, Xanthobacter, Xenorhabdus, Yersinia species (including Y. pestis and Y. enter ocoliticd), Zanthomonas and Zymomonas.

The clinical diseases or infections caused by Gram-positive and/or Gram-negative bacteria, treatable with the present disclosure include abscesses, bacteremia, contamination of peritoneal dialysis fluid, endocarditis, pneumonia, meningitis, osteomyelitis, cellulitis, pharyngitis, otitis media, sinusitis, scarlet fever, arthritis, urinary tract infection, laryngotracheitis, erysipeloid, gas gangrene, tetanus, typhoid fever, acute gastroenteritis, bronchitis, epiglottitis, plague, sepsis, chancroid, wound and burn infection, cholera, glanders, periodontitis, genital infections, empyema, granuloma inguinale, Legionnaire's disease, paratyphoid, bacillary dysentary, brucellosis, diphtheria, pertussis, botulism, toxic shock syndrome, mastitis, rheumatic fever, cystic fibrosis, eye infections, plaque, and dental caries. Other uses include swine erysipelas, peritonitis, abortion, encephalitis, anthrax, nocardiosis, pericarditis, mycetoma, peptic ulcer, melioidosis, HaverhiU fever, tularemia, Moko disease, galls (e.g., crown, cane and leaf), hairy root, bacterial rot, bacterial blight, bacterial brown spot, bacterial wilt, bacterial fin rot, dropsy, columnaris disease, pasteurellosis, furunculosis, enteric redmouth disease, vibriosis offish, and fouling of medical devices.

Compounds and compositions of the present disclosure may be effective against influenza virus, cytomegalovirus, avian leukosis-sarcoma virus, Rous Sarcoma virus, Mammalian C-type Murine leukemia virus, Feline leukemia virus, simian sarcoma virus, B-type Mouse mammary tumor virus, D-type virus Mason-Pfizer monkey virus, simian AIDS virus, Human T-cell leukemia virus, Simian T-cell leukemia virus, bovine leukemia virus, Human immunodeficiency virus, Simian immunodeficiency virus, Feline immunodeficiency virus, Visna/maedi virus, Equine infectious anemia virus, Caprine arthritis-encephalitis virus, spumavirus, foamy virus, endogenous retrovirus, papilloma virus, respiratory syncytial virus, poliomyelitis virus, pox virus, measles virus, arbor virus, Coxsackie virus, herpes virus, hantavirus, hepatitis virus, baculovirus, mumps virus, circovirus, arenavirus, rotavirus, Colorado Tick Fever CTF virus, Eyach virus, Langat virus, Powassan virus, Omsk hemorrhagic fever virus, Crimean-Congo hemorrhagic fever virus, Yellow fever virus, Encephalitis virus, St. Louis Encephalitis virus, Venezuelan equine encephalitis virus, Western equine encephalitis virus, Chikungunya virus, Japanese encephalitis virus, West Nile virus, Kyasanur forest disease virus, Dengue fever virus, California encephalitis virus, adenovirus, Korean haemorrhagic fever virus, hantavirus, Argentine haemorrhagic fever virus, Junin virus, Aujeszky disease virus, Pseudorabies virus, Herpesvirus, Chikungunya virus, cowpox virus, ebolavirus, Ganjam virus, herpesvirus simiae, Lassa fever virus, Louping ill virus, Lymphocytic choriomeningitis virus, Marburg virus, Milkers nodule virus, Newcastle disease virus, Omsk haemorrhagic fever virus, Orf virus, Parvovirus, Poliovirus, Pseudorabies, Rabies virus, Rift Valley fever virus, Russian Spring-Summer encephalitis virus, Sabia virus, vaccinia virus, vesicular stomatitis virus, Western equine encephalitis virus, or Yellow fever virus.

### C. Autoimmune Diseases

Compounds and compositions of the present disclosure may be effective in the treatment or prevention of autoimmune disease. Non-limiting examples of auto-immune diseases include Addison's disease, Agammaglobulinemia, Alopecia areata, Amyloidosis, Ankylosing spondylitis, Anti-GBM/Anti-TBM nephritis, Antiphospholipid syndrome (APS), Autoimmune hepatitis, Autoimmune inner ear disease (AIED), Axonal & neuronal neuropathy (AMAN), Behcet's disease, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss, Cicatricial pemphigoid/benign mucosal pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hypogammalglobulinemia, IgA Nephropathy, IgG4-related sclerosing disease, Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multiple sclerosis (MS), Myasthenia gravis, Myositis, Narcolepsy, Neuromyelitis optica, Neutropenia, Ocular cicatricial pemphigoid, Optic neuritis, Palindromic rheumatism (PR), PANDAS (Pediatric Autoimmune Neuropsychiatric Disorders Associated with Streptococcus), Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome (polyneuropathy, organomegaly, endocrinopathy, monoclonal gammopathy, skin changes), Polyarteritis nodosa, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Reiter's syndrome, Relapsing polychondritis, Restless legs syndrome (RLS), Retroperitoneal fibrosis, Rheumatic fever, Rheumatoid arthritis (RA), Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Wegener's granulomatosis (now termed Granulomatosis with Polyangiitis (GPA).

### D. Inflammation

Inflammation is a biological process that provides resistance to infectious or parasitic organisms and the repair of damaged tissue. Inflammation is commonly characterized by localized vasodilation, redness, swelling, and pain, the recruitment of leukocytes to the site of infection or injury, production of inflammatory cytokines such as TNF-α and IL-1, and production of reactive oxygen or nitrogen species such as hydrogen peroxide, superoxide and peroxynitrite. In later stages of inflammation, tissue remodeling, angiogenesis, and scar formation (fibrosis) may occur as part of the wound healing process. Under normal circumstances, the inflammatory response is regulated and temporary and is resolved in an orchestrated fashion once the infection or injury has been dealt with adequately. However, acute inflammation can become excessive and life-threatening if regulatory mechanisms fail. Alternatively, inflammation can become chronic and cause cumulative tissue damage or systemic complications.

Many serious and intractable human diseases involve dysregulation of inflammatory processes, including diseases such as cancer, atherosclerosis, and diabetes, which were not traditionally viewed as inflammatory conditions. In the case of cancer, the inflammatory processes are associated with tumor formation, progression, metastasis, and resistance to therapy. Atherosclerosis, long viewed as a disorder of lipid metabolism, is now understood to be primarily an inflammatory condition, with activated macrophages playing an important role in the formation and eventual rupture of atherosclerotic plaques. Activation of inflammatory signaling pathways has also been shown to play a role in the development of insulin resistance, as well as in the peripheral tissue damage associated with diabetic hyperglycemia

Chronic organ failure such as renal failure, heart failure, liver failure, and chronic obstructive pulmonary disease is closely associated with the presence of chronic oxidative stress and inflammation, leading to the development of fibrosis and eventual loss of organ function. Oxidative stress in vascular endothelial cells, which line major and minor blood vessels, can lead to endothelial dysfunction and is believed to be an important contributing factor in the development of systemic cardiovascular disease, complications of diabetes, chronic kidney disease and other forms of organ failure, and a number of other aging-related diseases including degenerative diseases of the central nervous system and the retina.

Many other disorders involve oxidative stress and inflammation in affected tissues, including inflammatory bowel disease; inflammatory skin diseases; mucositis related to radiation therapy and chemotherapy; eye diseases such as uveitis, glaucoma, macular degeneration, and various forms of retinopathy; transplant failure and rejection; ischemia-reperfusion injury; chronic pain; degenerative conditions of the bones and joints including osteoarthritis and osteoporosis; asthma and cystic fibrosis; seizure disorders; and neuropsychiatric conditions including schizophrenia, depression, bipolar disorder, post-traumatic stress disorder, attention deficit disorders, autism-spectrum disorders, and eating disorders such as anorexia nervosa. Dysregulation of inflammatory signaling pathways is believed to be a major factor in the pathology of muscle wasting diseases including muscular dystrophy and various forms of cachexia.

A variety of life-threatening acute disorders also involve dysregulated inflammatory signaling, including acute organ failure involving the pancreas, kidneys, liver, or lungs, myocardial infarction or acute coronary syndrome, stroke, septic shock, trauma, severe burns, and anaphylaxis.

Many complications of infectious diseases also involve dysregulation of inflammatory responses. Although an inflammatory response can kill invading pathogens, an excessive inflammatory response can also be quite destructive and in some cases can be a primary source of damage in infected tissues. Furthermore, an excessive inflammatory response can also lead to systemic complications due to overproduction of inflammatory cytokines such as TNF-α and IL-1. This is believed to be a factor in mortality arising from severe influenza, severe acute respiratory syndrome, and sepsis.

### V. CELL TARGETING MOIETIES

In some aspects, the present disclosure provides compounds conjugated directly or through linkers to a cell targeting moiety. In some embodiments, the conjugation of the compound to a cell targeting moiety increases the efficacy of the compound in treating a disease or disorder. Cell targeting moieties according to the embodiments may be, for example, an antibody, a growth factor, a hormone, a peptide, an aptamer, a small molecule such as a hormone, an imaging agent, or cofactor, or a cytokine. It has been demonstrated that the gp240 antigen is expressed in a variety of melanomas but not in normal tissues. Thus, in some embodiments, the compounds of the present disclosure may be used in conjugates with an antibody for a specific antigen that is expressed by a cancer cell but not in normal tissues.

In certain additional embodiments, it is envisioned that cancer cell targeting moieties bind to multiple types of cancer cells. For example, the 8H9 monoclonal antibody and the single chain antibodies derived therefrom bind to a glycoprotein that is expressed on breast cancers, sarcomas and neuroblastomas (Onda et al., 2004). Another example is the cell targeting agents described in U.S. Patent Publication No. 2004/0005647 and in Winthrop et al. (2003) that bind to MUC-1, an antigen that is expressed on a variety cancer types. Thus, it will be understood that in certain embodiments, cell targeting constructs according the embodiments may be targeted against a plurality of cancer or tumor types.

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the cell-specific targeting moieties in cancer therapy. Additionally, the cell targeting moiety that may be used include a cofactor, a sugar, a drug molecule, an imaging agent, or a fluorescent dye. Many cancerous cells are known to over express folate receptors and thus folic acid or other folate derivatives may be used as conjugates to trigger cell-specific interaction between the conjugates of the present disclosure and a cell (Campbell et al., 1991; Weitman et al., 1992).

Since a large number of cell surface receptors have been identified in hematopoietic cells of various lineages, ligands or antibodies specific for these receptors may be used as cell-specific targeting moieties. IL-2 may also be used as a cell-specific targeting moiety in a chimeric protein to target IL-2R+ cells. Alternatively, other molecules such as B7-1, B7-2 and CD40 may be used to specifically target activated T cells (The Leucocyte Antigen Facts Book, 1993, Barclay et al. (eds.), Academic Press). Furthermore, B cells express CD19, CD40 and IL-4 receptor and may be targeted by moieties that bind these receptors, such as CD40 ligand, IL-4, IL-5, IL-6 and CD28. The elimination of immune cells such as T cells and B cells is particularly useful in the treatment of lymphoid tumors.

Other cytokines that may be used to target specific cell subsets include the interleukins (IL-1 through IL-15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego). In some aspects, the targeting polypeptide is a cytokine that binds to the Fn14 receptor, such as TWEAK (see, e.g., Winkles 2008; Zhou et al., 2011 and Burkly et al., 2007, incorporated herein by reference).

A skilled artisan recognizes that there are a variety of known cytokines, including hematopoietins (four-helix bundles) (such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)); interferons (such as IFN-γ, IFN-α, and IFN-β); immunoglobin superfamily (such as B7.1 (CD80), and B7.2 (B70, CD86)); TNF family (such as TNF-α (cachectin), TNF-β (lymphotoxin, LT, LT-α), LT-β, CD40 ligand (CD40L), Fas ligand (FasL), CD27 ligand (CD27L), CD30 ligand (CD30L), and 4-1BBL)); and those unassigned to a particular family (such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)). Furthermore, the Fc portion of the heavy chain of an antibody may be used to target Fc receptor-expressing cells such as the use of the Fc portion of an IgE antibody to target mast cells and basophils.

Furthermore, in some aspects, the cell-targeting moiety may be a peptide sequence or a cyclic peptide. Examples, cell- and tissue-targeting peptides that may be used according to the embodiments are provided, for instance, in U.S. Pat. Nos. 6,232,287; 6,528,481; 7,452,964; 7,671,010; 7,781,565; 8,507,445; and 8,450,278, each of which is incorporated herein by reference.

Thus, in some embodiments, cell targeting moieties are antibodies or avimers. Antibodies and avimers can be generated against virtually any cell surface marker thus, providing a method for targeted to delivery of GrB to virtually any cell population of interest. These antibodies could also be used as fragments. Furthermore, the antibodies could have been developed in one type of animal and then humanized or developed using a human model. Methods for generating antibodies that may be used as cell targeting moieties are detailed below. Methods for generating avimers that bind to a given cell surface marker are detailed in U.S. Patent Publications Nos. 2006/0234299 and 2006/0223114, each incorporated herein by reference.

Additionally, it is contemplated that the compounds described herein may be conjugated to a nanoparticle or other nanomaterial. Some non-limiting examples of nanoparticles include metal nanoparticles such as gold or silver nanoparticles or polymeric nanoparticles such as poly-L-lactic acid or poly(ethylene) glycol polymers. Nanoparticles and nanomaterials which may be conjugated to the instant compounds include those described in U.S. Patent Publications Nos. 2006/0034925, 2006/0115537, 2007/0148095, 2012/0141550, 2013/0138032, and 2014/0024610 and PCT Publication No. 2008/121949, 2011/0053435, and 2014/0087413, each incorporated herein by reference.

### VI. THERAPIES

### A. Pharmaceutical Formulations and Routes of Administration

Where clinical applications are contemplated, it will be necessary to prepare pharmaceutical compositions in a form appropriate for the intended application. In some embodiments, such formulation with the ADCs of the present disclosure is contemplated. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the ADCs described herein may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA's Division of Biological Standards and Quality Control of the Office of Compliance and Biologics Quality.

### B. Methods of Treatment

In particular, the compositions that may be used in treating a disease or disorder, such as cancer, in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells or killing microbes). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that induces death of cancerous cells (e.g., induces apoptosis of a cancer cell), as assayed by identifying a reduction in hematological parameters (complete blood count—CBC), or cancer cell growth or proliferation. In some embodiments, amounts of the ADCs used to induce apoptosis of the cancer cells is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the ADCs may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In some embodiments, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer (e.g., leukemia) in which the subject has been administered a therapeutic amount of a composition as described herein. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

### C. Combination Therapies

It is envisioned that the drug conjugates described herein may be used in combination therapies with one or more therapies or a compound which mitigates one or more of the side effects experienced by the patient. It is common in the field of medical therapy to combine therapeutic modalities. The following is a general discussion of therapies that may be used in conjunction with the therapies of the present disclosure.

To treat certain diseases or disorders using the methods and compositions of the present disclosure, one would generally contact the subject with a compound and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the drug conjugates described herein may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated.

### VII. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, within a standard deviation of the stated value as determined using a standard analytical technique for measuring the stated value. The terms can also be used by referring to plus or minus 5% of the stated value.

The phrase “effective amount” or “therapeutically effective” means a dosage of a drug or agent sufficient to produce a desired result. The desired result can be subjective or objective improvement in the recipient of the dosage, increased lung growth, increased lung repair, reduced tissue edema, increased DNA repair, decreased apoptosis, a decrease in tumor size, a decrease in the rate of growth of cancer cells, a decrease in metastasis, or any combination of the above.

As used herein, the term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The antibody may be a bi-specific antibody. In exemplary embodiments, antibodies used with the methods and compositions described herein are derivatives of the IgG class. The term antibody also refers to antigen-binding antibody fragments. Examples of such antibody fragments include, but are not limited to, Fab, Fabÿ, F(aby)₂, scFv, Fv, dsFv diabody, and Fd fragments. Antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 10 amino acids and more typically will comprise at least about 200 amino acids.

“Subject” and “patient” refer to either a human or non-human, such as primates, mammals, and vertebrates. In particular embodiments, the subject is a human.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof, “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

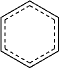

covers, for example,

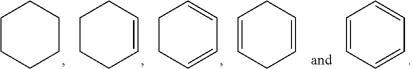

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

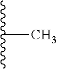

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

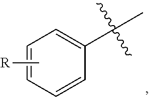

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

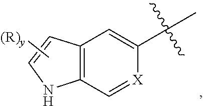

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom(s) in the moiety replacing a hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group refers to a planar unsaturated ring of atoms with 4n+2 electrons in a fully conjugated cyclic R system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ⁱBu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

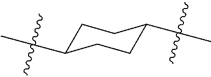 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structure, wherein the ring atoms are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

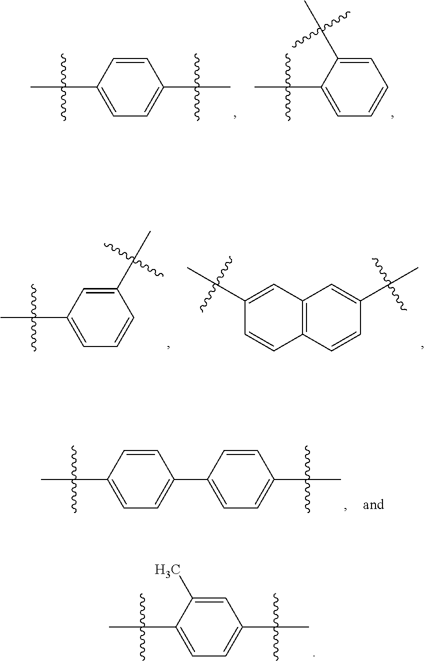

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroaryl does not preclude the presence of one or more alkyl or aryl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroarenediyl does not preclude the presence of one or more alkyl or aryl groups (carbon number limitation permitting) attached to the aromatic ring or aromatic ring system. Non-limiting examples of heteroarenediyl groups include:

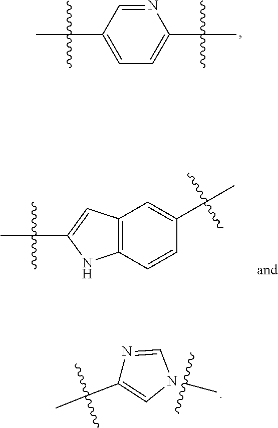

The term “heterocycloalkanediyl” when used without the “substituted” modifier refers to an divalent cyclic group, with two carbon atoms, two nitrogen atoms, or one carbon atom and one nitrogen atom as the two points of attachment, said atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings are connected with a covalent bond. As used herein, the term heterocycloalkanediyl does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

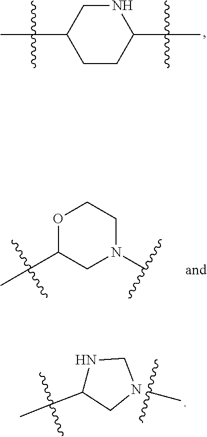

The terms “alkylsulfonyl” and “alkylsulfinyl” when used without the “substituted” modifier refers to the groups —S(O)₂R and —S(O)R, respectively, in which R is an alkyl, as that term is defined above. The terms “cycloalkylsulfonyl”, “alkenylsulfonyl”, “alkynylsulfonyl”, “arylsulfonyl”, “aralkylsulfonyl”, “heteroarylsulfonyl”, and “heterocycloalkylsulfonyl” are defined in an analogous manner. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “dialkylphosphate” when used without the “substituted” modifier refers to the group —OP(O)(OR)(OR′), in which R and R′ can be the same or different alkyl groups, or R and R′ can be taken together to represent an alkanediyl. Non-limiting examples of dialkylphosphate groups include: —OP(O)(OMe)₂, —OP(O)(OEt)(OMe) and —OP(O)(OEt)₂. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical agent, pharmaceutical preparation, pharmaceutical composition, pharmaceutical formulation, pharmaceutical product, medicinal product, medicine, medication, medicament, or simply a drug) is a drug used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

The “polyvalent polymer” describes a linking group which contains two or more open valent points that can be used to connect different components together. Some non-limiting examples of polyvalent polymers include polymers with side chains that are capable of reacting with the component, dendrimers, dendrons, or fragments thereof. In one embodiment, the polyvalent polymer is a dendrimer or dendron.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

An “amine protecting group” is well understood in the art. An amine protecting group is a group which prevents the reactivity of the amine group during a reaction which modifies some other portion of the molecule and can be easily removed to generate the desired amine. Amine protecting groups can be found at least in Greene and Wuts, 1999, which is incorporated herein by reference. Some non-limiting examples of monovalent amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH— or PG_{DA}N— wherein PG_{MA} is a monovalent amine protecting group, which may also be described as a “monvalently protected amino group” and PG_{DA} is a divalent amine protecting group as described above, which may also be described as a “divalently protected amino group”.

A “hydroxyl protecting group” is well understood in the art. A hydroxyl protecting group is a group which prevents the reactivity of the hydroxyl group during a reaction which modifies some other portion of the molecule and can be easily removed to generate the desired hydroxyl. Hydroxyl protecting groups can be found at least in Greene and Wuts, 1999, which is incorporated herein by reference. Some non-limiting examples of hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. When used herein, a protected hydroxy group is a group of the formula PG_{H}O— wherein PG_{H} is a hydroxyl protecting group as described above.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein, average molecular weight refers to the weight average molecular weight (Mw) determined by static light scattering.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc.

The term “conjugating group” refers to a chemical group capable of coupling with the functional group of another compound to form a covalent bond under mild conditions and which is stable under physiological conditions. In some embodiments, the conjugating group can form the covalent bond via an S_{N}2 reaction, a Diels-Alder reaction, or a conjugate addition reaction. Examples of conjugating groups include, but are not limited to, diene groups (such as tetrazinyl), alkene groups (such as trans-cyclooctenyl and norbornyl), and —SH.

The term “canonical amino acid” refers to one of the 20 standard amino acids using in nature. Other amino acids include citrillune or ornithine. The term that “the side chain” of an amino acid refers to R group on the particular amino acid. For example, the R group of glycine is hydrogen atom, alanine is a methyl group, and lysine is a 4-aminobutyl.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VIII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Development and Characterization of Tripeptide Linkers

The present studies showed that glutamic acid-glycine-citrulline (EGCit) tripeptide linkers have the potential to solve the clinical issues caused by linker instability without compromising ADC therapeutic efficacy (FIG. 1C). The EGCit sequence provides long-term stability in both rodent and primate plasma and completely spares differentiating human neutrophils, while retaining the capacity to quickly liberate free payloads upon intracellular cleavage. It was also demonstrated that MMAE ADCs constructed with EGCit linkers exhibit improved antitumor activity in a panel of cancer cell lines and three different xenograft mouse models compared to conventional conjugates as well as FDA-approved anti-HER2 ADCs KADCYLA® (T-DM1) and ENHERTU® (DS-8201). Notably, the EGCit-based ADC showed no discernable liver toxicity in healthy mice at 80 mg kg⁻¹. These findings indicate that the EGCit linker technology not only ensures smooth transition from preclinical studies to in-human evaluation, but also provides a broadly applicable solution for substantially widening therapeutic windows of targeted drug delivery systems including ADCs.

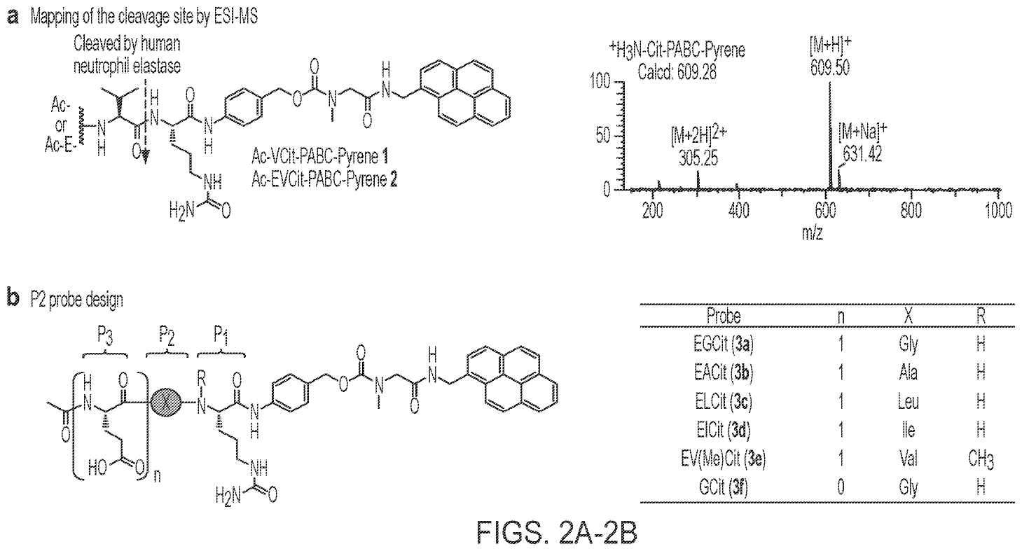

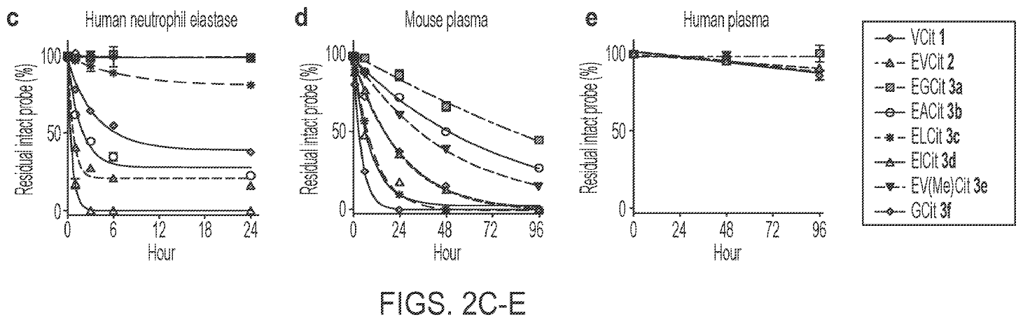

Incorporation of glycine at P2 and glutamic acid at P3 afforded high resistance to undesired degradation. To seek alternatives to the conventional VCit linker with improved in vivo stability and tolerability, it was first investigated how the linker instability leading to neutropenia could be circumvented. Serine proteases secreted extracellularly from differentiating human neutrophils have been shown to promote the release of MMAE from VCit-based ADCs, reducing the population of bone marrow neutrophils. (Zhao et al., 2017). With this report in mind, the neutrophil elastase cleavage site was identified using small-molecule probes. VCit-PABC-pyrene probe 1 was incubated with human neutrophil elastase at 37° C. for 24 h. A pyrene fragment containing citrulline-PABC was detected as the major product in liquid chromatography (LC)-electrospray ionization-mass spectrometry (ESI-MS) (FIG. 2A). This result indicated that neutrophil elastase cleaved the amide bond between P1 valine and P2 citrulline. EVCit probe 2 also provided the same citrulline-containing fragment, suggesting that glutamic acid at P3 does not affect neutrophil elastase-mediated linker degradation.

Based on this structural analysis, various amino acids were screened at the P2 position. A panel of pyrene probes were prepared containing an EXCit-PABC unit where X is glycine (3a), alanine (3b), leucine (3c), and isoleucine (3d) (FIG. 2B). EV(N-Me)Cit-PABC-pyrene probe 3e and GCit dipeptide probe 3f were also prepared. Subsequently, all probes were tested for stability against human neutrophil elastase-mediated degradation (FIG. 2C). Surprisingly, EVCit probe 2 degraded more quickly than VCit probe 1, indicating that the P3 glutamic acid can increase the linker susceptibility to elastase-mediated degradation. EACit and EICit probes 3b and 3d also showed complete degradation. These results were consistent with a previous study demonstrating that human neutrophil elastase preferentially cleaved the N-terminus amide bonds of valine, alanine, and isoleucine. (Fu et al., 2018). In contrast, marginal or almost no degradation was observed for probes containing EGCit (3a), ELCit (3c), EV(N-Me)Cit (3e), or GCit probe (3f). This observation suggested that the P2 amino acid impacted the reactivity with neutrophil elastase much more significantly than the P3 amino acid. EGCit and EV(N-Me)Cit probes 3a and 3e were further tested for resistance to cleavage by other abundant proteases secreted by human neutrophils: human proteinase 3 and cathepsin G. The EGCit probe 3a was completely intact in the presence of either protease, while the EV(N-Me)Cit probe 3e was partially degraded by proteinase 3 (Table 1).

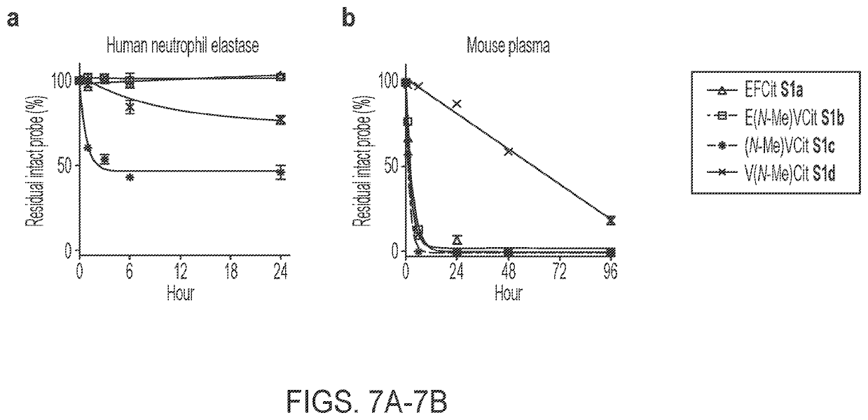

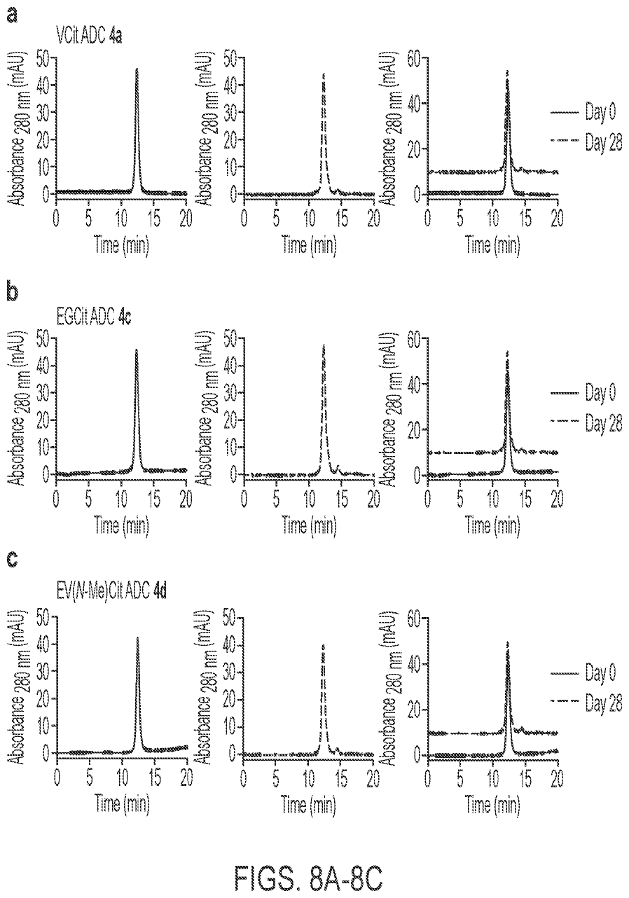

Next, probes 3a-f were tested for stability in undiluted BALB/c plasma (FIG. 2D). EGCit, EACit, and EV(N-Me)Cit probes 3a, 3b, and 3e showed improved stability compared to EVCit probe 2. In particular, the EGCit probe 3a probe retained 40% intact after a 4-day incubation. The GCit probe 3f was less stable than the EGCit probe 3a, which is consistent with the previous finding that glutamic acid at the P3 position enhances linker stability in mouse plasma. Furthermore, EGCit probe 3a probe completely withstood degradation in monkey and human plasma (FIG. 2E and Table 2). Finally, additional stability assays were performed for EFCit, E(N-Me)VCit, and V(N-Me)Cit probes. However, these probes showed instability against degradation mediated by neutrophil elastase or mouse plasma (FIG. 7) Collectively, these findings show that the EGCit sequence offers complete stability against undesired proteolytic degradation leading to premature payload release.

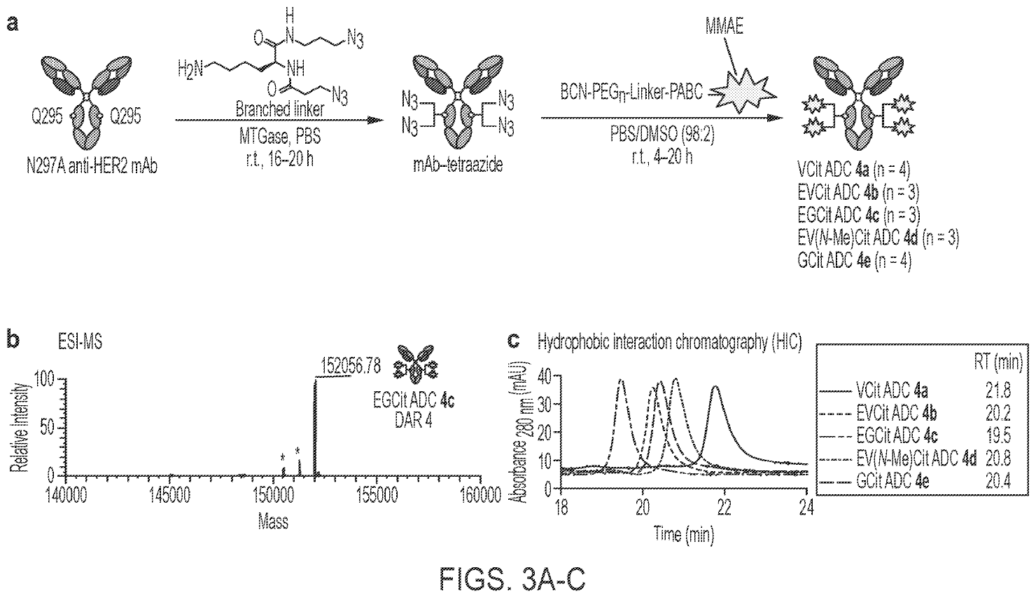

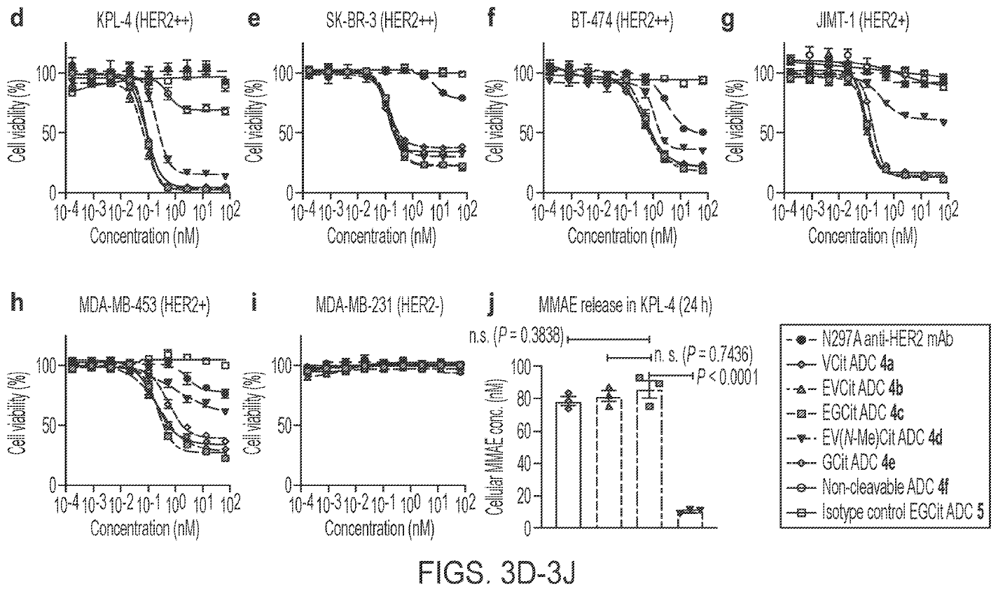

EGCit linker increases ADC hydrophilicity and cell killing potency with efficient intracellular payload release. Next, it was set out to investigate how the P2 amino acids evaluated above affected ADC physicochemical properties, intracellular payload release upon cleavage, and antigen-specific cell killing potency. To this end, anti-HER2 ADCs were constructed using selected P2-modified cleavable linkers and the conjugation technology previously developed (Amani et al., 2017; Anami et al., 2020; Anami et al., 2017) (FIG. 3A). First, diazide branched linkers were site-specifically installed onto the side chain of glutamine 295 (Q295) within N297A anti-HER2 mAb (derived from trastuzumab) by microbial transglutaminase (MTGase)-mediated transpeptidation. In parallel, VCit, EVCit, EGCit, EV(N-Me)Cit, and GCit linker-based modules containing bicyclo[6.1.0]nonyne (BCN) were prepared as a handle for strain-promoted azide-alkyne cycloaddition, polyethylene glycol (PEG), PABC as a self-immolative spacer, and MMAE. Finally, these payload modules underwent the click reaction with the mAb-tetraazide to afford homogeneous anti-HER2 ADC 4a-e with a drug-to-antibody ratio (DAR) of 4. The homogeneity was confirmed by ESI-MS analysis (FIG. 3B). SEC analysis confirmed that no significant degradation or aggregation occurred after incubating each ADC in phosphate buffered saline (PBS, pH 7.4) at 37° C. for 28 days (FIG. 2). Subsequently, hydrophobic interaction chromatography (HIC) analysis was performed under physiological conditions (phosphate buffer, pH 7.4) to assess the relative hydrophobicity of each ADC (FIG. 2C). EGCit ADC 4c was the least hydrophobic among the ADCs tested. EVCit ADC 4b, EV(N-Me)Cit ADC 4d, and GCit ADC 4e had intermediate hydrophobicity. VCit ADC 4a was the most hydrophobic. This result suggested that incorporating the smallest amino acid glycine at the P2 position and negatively charged glutamic acid at the P3 position can synergistically reduce ADC hydrophobicity at physiological pH. This feature is an advantage in the construction of ADCs because hydrophobic ADCs often show high aggregation rates leading to fast clearance (Lyon et al., 2015).

These ADCs were then evaluated for in vitro cytotoxicity in HER2-positive (KPL-4, SK-BR-3, BT-474, JIMT-1, MDA-MB-453) and -negative (MDA-MB-231) human breast cancer cell lines (FIGS. 3D-I). As controls, non-cleavable anti-HER2 MMAE ADC 4f (DAR 4) and non-targeting ADC constructed using BCN-EGCit-PABC-MMAE module (5, DAR 4) were also prepared and tested. VCit ADC 4a, EVCit ADC 4b, and EGCit ADC 4c exhibited comparable cell killing potency in the HER2-positive lines, but not in HER2-negative MDA-MB-231 cells; under the assay conditions, the ranges of the EC₅₀ values were 0.071-0.087 nM in KPL-4, 0.119-0.175 nM in SK-BR-3, 0.459-0.578 nM in BT-474, 0.088-0.114 nM in JIMT-1, and 0.183-0.254 nM in MDA-MB-453 (Table 3). These ADCs also showed similar maximum cell killing potency at high concentrations (Table 4). EV(N-Me)Cit ADC 4d exhibited a similar EC₅₀ value in SK-BR-3. However, its EC₅₀ values and cell viability at high concentrations were slightly or significantly greater than those of EGCit ADC 4c in the other HER2-positive cell lines. GCit ADC 4e were as potent as EGCit 4c in KPL-4 and JIMT-1. However, an increased EC₅₀ value and/or percentage of viable cells at the maximum ADC concentration were observed in SK-BR-3 and MDA-MB-453. Non-cleavable anti-HER2 ADC 4f, which lacked a peptide cleavable sequence within the linker scaffold, showed far greater EC₅₀ values in KPL-4 and JIMT-1 cells compared to cleavable ADCs 4a-c. Furthermore, both non-cleavable ADC 4f and non-targeting EGCit ADC 5 showed very poor or almost no cell killing effect in either cell line. These findings highlight the importance of internalization and following intracellular release of free MMAE for effective cell killing. The comparable potency of ADCs 4a-c also suggested that the EGCit linker has an enzymatic cleavage rate similar with those of VCit and EVCit linkers. To further verify this point, free MMAE released from ADC 4a-d in KPL-4 cells was quantified (FIG. 3J). After treating KPL-4 cells with each ADC for 24 h, the MMAE concentrations in cell lysates were determined by high-resolution MS. As anticipated, about 80% of conjugated MMAE was detected as a free payload in the VCit, EVCit, and EGCit ADCs 4a-c. In contrast, only 10.5% MMAE release was observed for EV(N-Me)Cit ADC 4d, indicating that the N-methylation of the citrulline retarded enzymatic linker cleavage necessary for payload release. Taken together, these results demonstrate that the hydrophilic EGCit sequence enables efficient traceless payload release upon ADC internalization in a wide range of cell types with varying catabolic profiles, ensuring maximal ADC potency.

EGCit ADC is stable in plasma and spares human neutrophils derived from the bone marrow. To assess ADC stability and safety profiles, ADCs 4a-c were first tested for plasma stability. No significant degradation was observed in either ADC after a 28-day incubation in undiluted human and monkey plasma at 37° C. (FIGS. 4A-4B and Tables 5-6). EVCit and EGCit ADCs 4b and 4c showed almost no linker cleavage after a 14-day incubation in undiluted BALB/c mouse plasma (FIG. 4C and Table 7). In contrast, VCit ADC 4a lost approximately 74% of the conjugated MMAE after the same period of time. Next, the stability of these ADCs was evaluated in the presence of human neutrophil elastase (FIG. 4D). EVCit ADCs 4b underwent partial degradation and DAR 0-3 fragments were generated, while EGCit ADC 4c completely resisted the degradation. These results are consistent with the earlier studies using pyrene probes (FIGS. 2C-E).

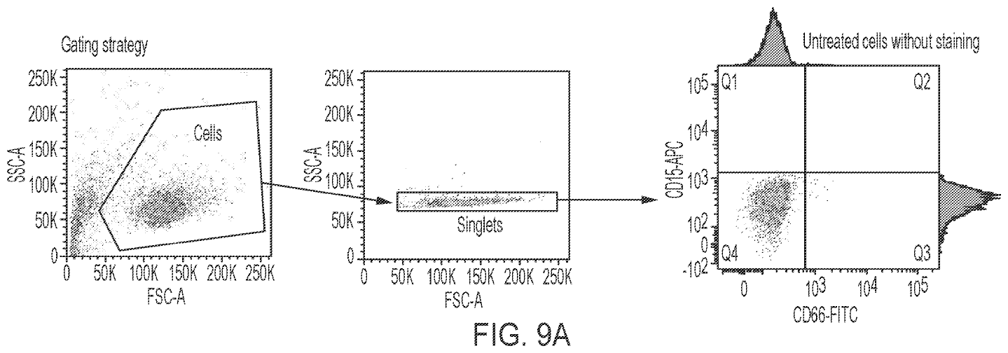

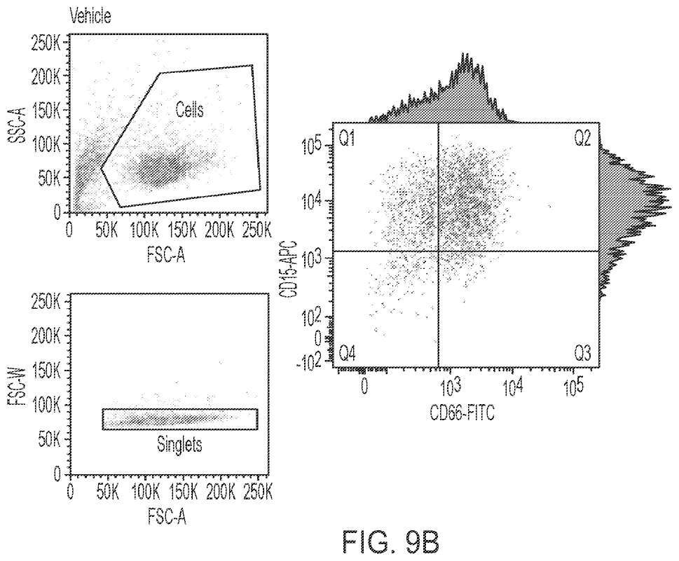

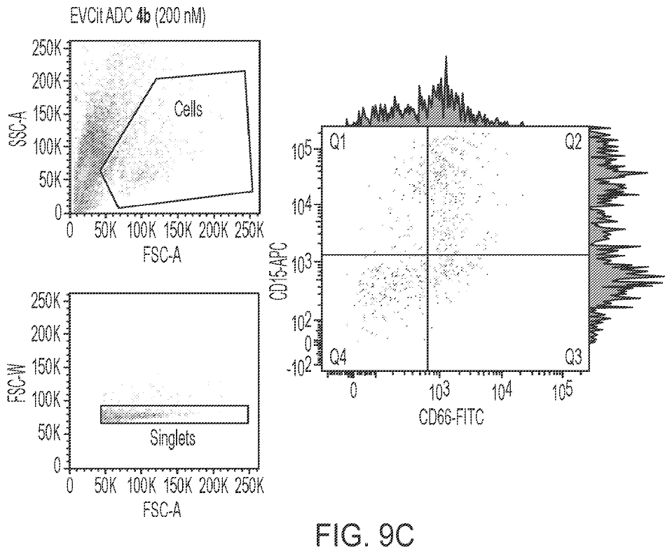

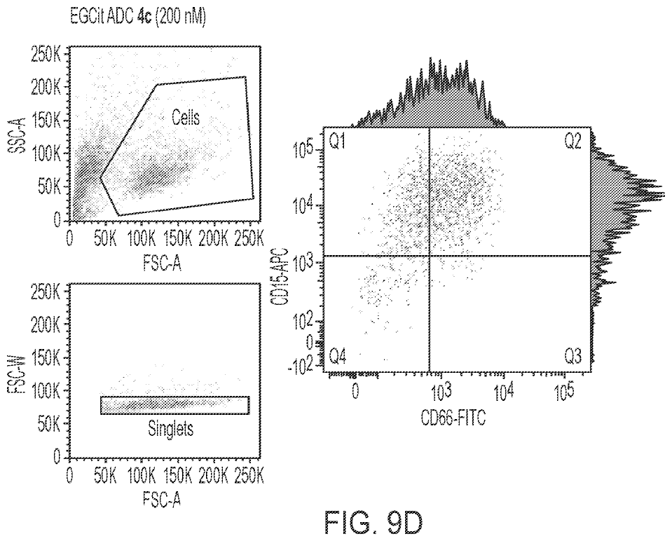

To investigate the potential effect of the ADCs on neutrophil production in the human bone marrow, ex vivo differentiation of hematopoietic stem and progenitor cells (HSPCs) into neutrophils was performed and following treatment with the ADCs (FIG. 4E). Zhao et al. reported that the population of differentiating human neutrophils markedly decreased by ADCs equipped with MMAE via a VCit linker while mature neutrophils could tolerate the toxic effect (Zhao et al., 2018). Following their protocol with some modifications, HSPCs collected from a single donor were differentiated into granulocytes with growth factors over a period of 7 days. At this point, the population of viable cells expressing both granulocyte markers CD15 and CD66b increased from 0.7% to 71.3% (FIGS. 4F, 4G, and 9). Because neutrophils are the most abundant granulocyte type in humans (50-75% of all leukocytes), these CD15/CD66b double-positive cells were considered as a population representing differentiating neutrophils. These cells were then treated with 200 nM of EVCit and EGCit ADCs 4b and 4c. Gratifyingly, EGCit ADC 4c caused only slight reduction in the percentage of CD15+/CD66+ cells relative to that in the vehicle control (FIGS. 4G and 9). In contrast, the neutrophil population significantly decreased to 46% by treatment with EVCit ADC 4b. A marked difference was also observed in non-specific toxicity to all hematopoietic cells between these two ADCs (FIG. 4I). The percentage of total viable cell counts (including both neutrophils and other cells) relative to that in the vehicle group sharply decreased to 8% by EVCit ADC 4b, while EGCit ADC 4c caused only moderate toxicity (relative viability: 45%). In the report by Zhao et al., such non-specific toxicity was observed for any ADCs at high concentrations (about 50% cell death at 10 nM) regardless of linker cleavability or cell permeability of payloads (Zhao et al., 2018). Collectively, these results highlight the potential of the EGCit linker to reduce the risk of myelosuppression, in particular neutropenia caused by prematurely released ADC payloads.

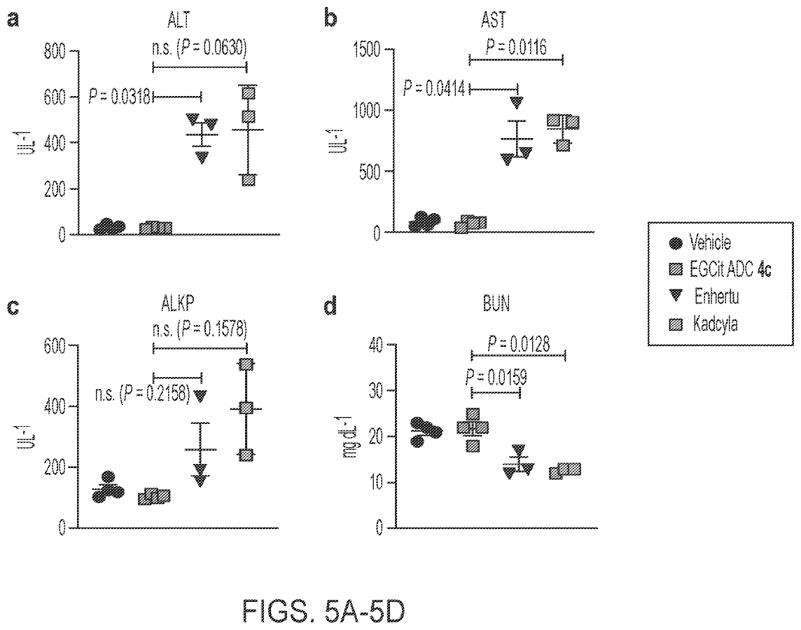

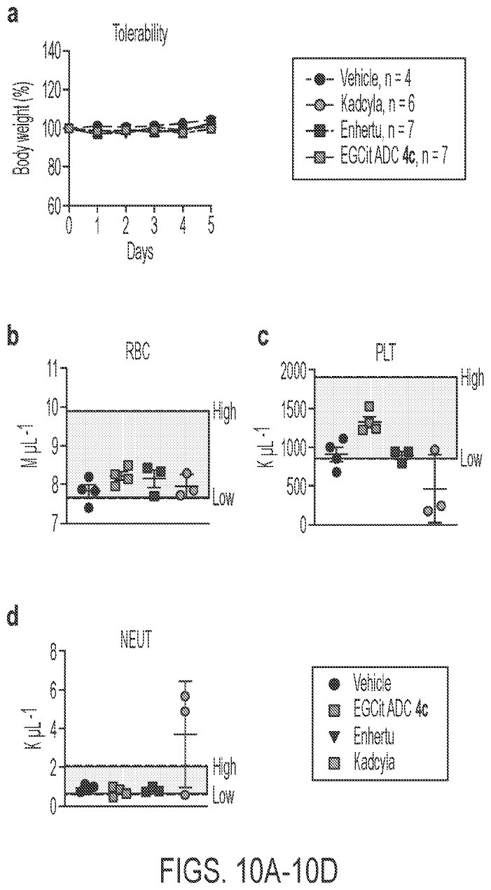

EGCit linker has the potential to minimize antigen-independent liver toxicity of ADCs. To investigate whether or not the EGCit linker impacted ADC safety at therapeutic doses, an exploratory study was performed in healthy CD-1® IGS mice using EGCit MMAE ADC 4c, EGCit MMA ENHERTU®, or KADCYLA® at 80 mg kg⁻¹. No significant body weight loss (>20%) or other severe clinical symptom was observed in either treatment group for 5 days post-injection (FIG. 10). Next, to evaluate potential liver toxicity, a blood chemistry test was performed by collecting serum at the end of the 5-day monitoring (FIGS. 5A-5D). The following molecules associated with liver functions were quantified: alanine aminotransferase (ALT), aspartate aminotransferase (AST), alkaline phosphatase (ALKP), and blood urea nitrogen (BUN). Increased AST, ALT, and ALKP and decreased BUN generally indicate liver damage. In the case of the EGCit ADC, the values of these parameters were comparable with those of the untreated mice. In contrast, mice treated with ENHERTU® and KADCYLA® showed significantly elevated levels of AST and ALT and a decreased level of BUN. The ALKP level also appeared to be elevated with these two ADCs. Hematology analysis was also performed using whole blood samples collected on Day 5. No significant changes in red blood cell, platelet, and neutrophil counts were observed for the present ADC or ENHERTU® compared to the untreated cohort, while KADCYLA® appeared to decrease platelets and increase neutrophils (FIG. 10). Overall, these results suggest that the EGCit MMAE ADC has improved safety profiles compared to current ADCs.

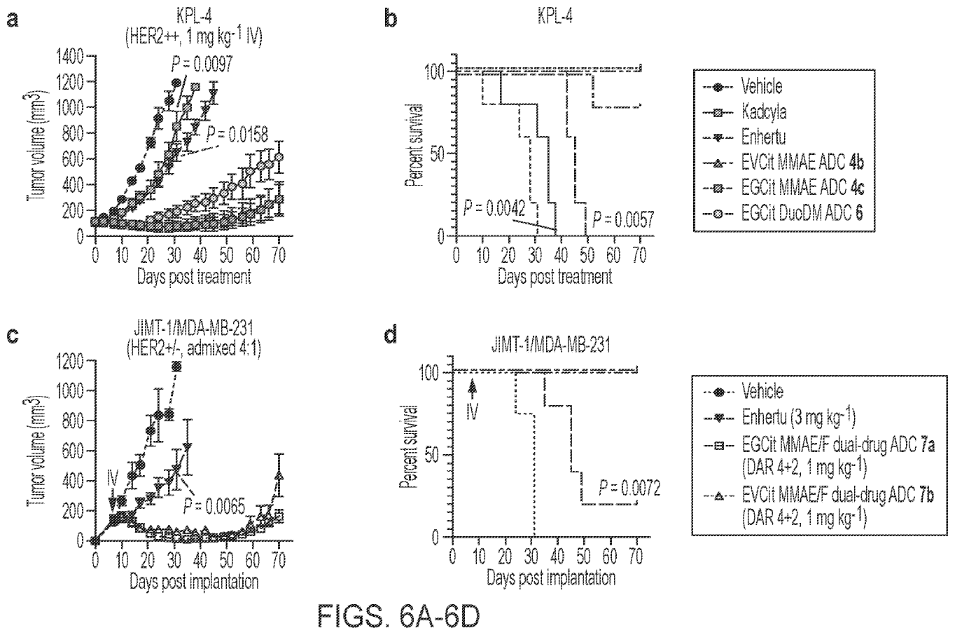

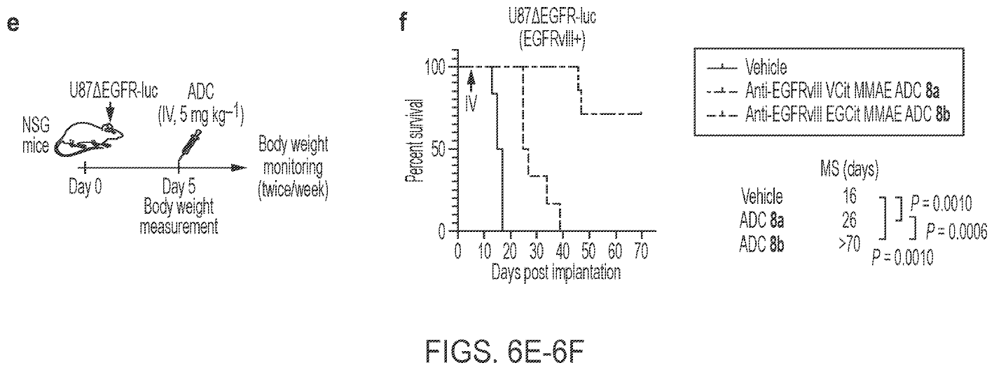

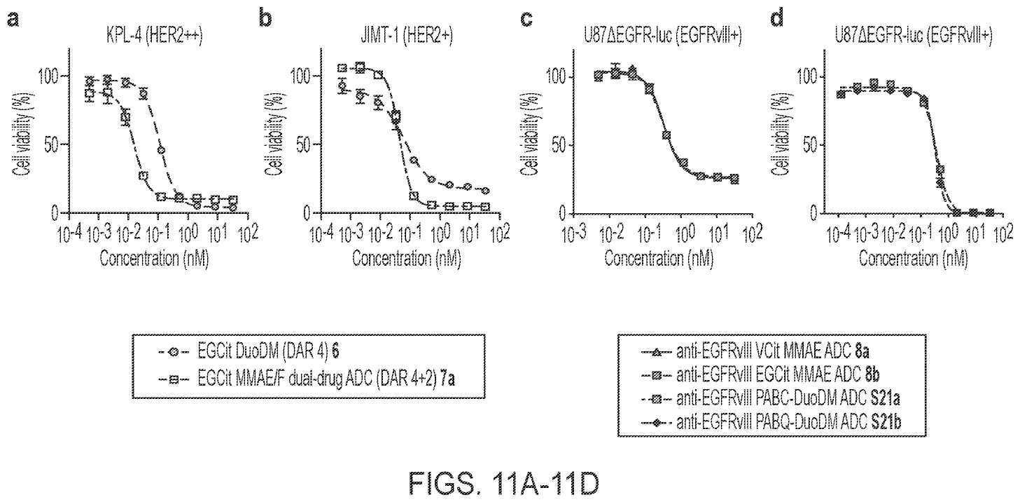

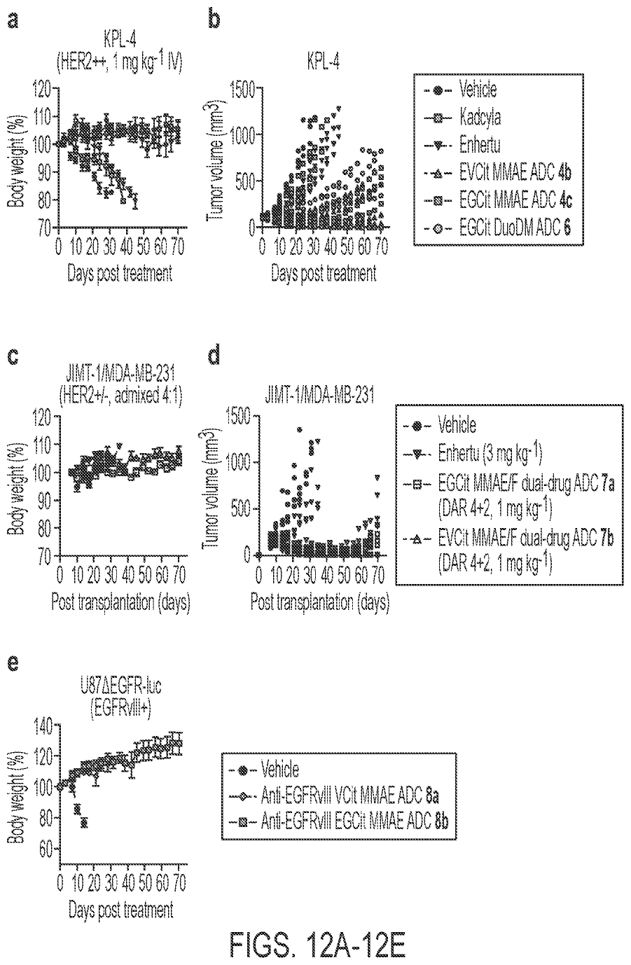

EGCit ADCs exert improved antitumor effects in various xenograft models compared to conventional ADCs. It was next sought to evaluate EGCit-based ADCs for treatment efficacy in multiple xenograft mouse tumor models (FIGS. 6A-6F). In the KPL-4 inflammatory breast tumor model, orthotopically xenografted NOD-scid gamma (NSG) mice were injected intravenously with a single dose of our anti-HER2 ADC 4b or 4c, KADCYLA®, or ENHERTU® at 1 mg kg⁻¹. To examine the applicability of the EGCit linker technology, duocarmycin DM (DuoDM) ADCs 6 were also prepared. This ADC showed sub-nanomolar in vitro EC₅₀ values (0.116 nM in KPL-4 and 0.059 nM in JIMT-1) as seen for MMAE ADC 4c, demonstrating that the EGCit linker can also release DuoDM efficiently (FIG. 11 and Table 3). No significant toxicity associated with administration of ADCs 4b, 4c and 6 was observed over the course of study, as evaluated by monitoring significant body weight loss and other clinical symptoms (FIG. 12). In addition, these ADCs exhibited remarkable percent tumor growth inhibition (% TGI on Day 31: 107%, ADC 4b; 104%, ADC 4c; 94%, ADC 6) and survival benefits (animal death by Day 70: 0 out of 5, 4b, c; 1 out of 5, 6) (FIGS. 6A-6B and 12). In contrast, compared to EGCit ADC 4c, only limited tumor growth inhibition was observed for KADCYLA® (47% TGI, P=0.0097) and ENHERTU® (48% TGI, P=0.0158). All animals treated with these FDA-approved ADCs were found dead or killed at the pre-defined humane endpoint (>1,000 mm³ tumor size or >20% body weight loss) by the end of the study (median survival time: 35 days, KADCYLA®, 45 days, ENHERTU®).

Next, the clinical potential of the EGCit linker was tested in the dual-drug ADC format. It has been demonstrated that a dual-drug ADC equipped with MMAE and MMAF can effectively treat low-HER2 heterogeneous breast tumors with elevated drug resistance (Yamazaki et al., 2021). EGCit-based MMAE/F DAR 4+2 ADC 7a was prepared. This ADC and ENHERTU® were tested in the JIMT-1/MDA-MB-231 admixed tumor model established by us, a model of refractory human breast cancer characterized by aggressive growth, heterogeneous HER2 expression, and moderate resistance to hydrophobic payloads such as MMAE (Yamazaki et al., 2021). A single dose of each ADC (ENHERTU®, 3 mg kg⁻¹; ADCs 7a, 1 mg kg⁻¹) was intravenously administered to orthotopic tumor-bearing nude mice 8 days post-implantation (average tumor volume: 100-150 mm³). No acute toxicity associated with ADC administration was observed for either ADC (FIG. 12). EGCit dual-drug ADC 7a exhibited remarkable antitumor effect (112% TGI on Day 31, FIGS. 6C and 12) and survival benefits (no animal death by Day 70, FIG. 6D). In contrast, even with a dose increase to 3 mg kg⁻¹, ENHERTU® exhibited only moderate inhibition of tumor growth in this HER2 heterogeneous tumor model (76% TGI on Day 31, P=0.0065, comparison control: EGCit ADC 7a). Further, 4 out of 5 mice needed to be euthanized by the end of the study due to severe clinical symptoms including ulceration.

Finally, it was set out to establish the generalizability of our linker technology by testing in an orthotopic glioblastoma multiforme (GBM) model (FIGS. 6E-6F). ADC-based systemic treatment of GBM has been unsuccessful as demonstrated by the recent failure of depatuxizumab mafodotin (Depatux-M, formerly called ABT-414) (Phillips et al., 2016) and AMG-595 (Hamblett et al., 2015) in clinical trials. Using the present linker technology, we could construct a panel of homogeneous anti-epidermal growth factor receptor variant III (EGFRvIII) ADCs from N297A depatuxizumab, including conventional VCit ADC 8a, EGCit ADC 8b, and EGCit-DuoDM ADCs conjugated via PABC or p-aminobenzyl quaternary ammonium (PABQ) linkage (Staben et al., 2016). These ADCs were equally potent in EGFRvIII-positive U87ΔEGFR-luc GBM cells (FIG. 11). ADCs 8a and b were then tested in the orthotopic U87ΔEGFR-luc model (FIG. 6E). Intracranial tumor-bearing NSG mice were injected intravenously with a single dose of each ADC at 5 mg kg⁻¹ 5 days post-implantation. No acute toxicity associated with ADC administration was observed in either group (FIG. 12). The short survival time of the untreated cohort (median survival time: 16 days) demonstrated the extremely aggressive tumor growth of this model (FIG. 6F). EGCit ADC 8b exerted remarkable therapeutic efficacy; the medium survival time was extended to >70 days, and 5 out of 7 mice achieved complete remission without any clinical symptom at the end of the study. In contrast, VCit ADC 8a moderately extended median survival time of 26 days (P=0.001, vs vehicle; P=0.0006, vs EGCit ADC 8b). All mice treated with VCit ADC 8a died or euthanized by the end of study.

TABLE 1
Stability of pyrene-based peptide probes in the presence of human
neutrophil enzymes. Quantification was performed after incubation
for 24 h (n = 2). Data are presented as mean values ± SEM.

| | % intact probe | | |
|---|---|---|---|
| Probe | Neutrophil elastase | Proteinase 3 | Cathepsin G |
| VCit 1 | 39.9 ± 3.8 | 67.9 ± 2.8 | 83.5 ± 2.8 |
| EVCit 2 | 14.3 ± 1.9 | 6.5 ± 0.1 | 63.0 ± 0.5 |
| EGCit 3a | 99.2 ± 0.3 | 98.0 ± 1.1 | 100.0 ± 1.8 |
| EV(N-Me)Cit | 99.0 ± 2.4 | 84.4 ± 0.9 | 99.6 ± 0.3 |
| 3e | | | |

TABLE 2
Stability of pyrene-based peptide probes in undiluted
human, monkey, and mouse plasma. Quantification was
performed after incubation for 48 h and 96 h (n =
2). Data are presented as mean values ± SEM.

| | % intact probe | | | | | |
|---|---|---|---|---|---|---|
| | 48 h incubation | | | 96 h incubation | | |
| Probe | Human | Monkey | Mouse | Human | Monkey | Mouse |
| VCit 1 | 97.3 ± | 91.5 ± | 0 | 86.2 ± | 79.8 ± | 0 |
| | 0.2 | 1.3 | | 2.5 | 3.1 | |
| EVCit 2 | 95.1 ± | 95.8 ± | 13.0 ± | 91.1 ± | 95.8 ± | 0 |
| | 0.3 | 3.9 | 0.5 | 1.1 | 1.8 | |
| EGCit 3a | 96.5 ± | 98.7 ± | 69.2 ± | 100.3 ± | 98.8 ± | 40.3 ± |
| | 1.7 | 0.7 | 0.9 | 5.5 | 1.6 | 3.8 |
| EV(N-Me)Cit | 99.5 ± | 97.8 ± | 37.8 ± | 86.4 ± | 91.9 ± | 12.0 ± |
| 3e | 0.2 | 0.9 | 0.6 | 3.2 | 1.2 | 2.9 |

TABLE 3
EC₅₀ values in human breast cancer cell lines (n = 4) and a GBM cell line
(n = 3). Values in parentheses are 95% confidential intervals.

| | EC₅₀ (nM) | | | | | | |
|---|---|---|---|---|---|---|---|
| | | SK-BR- | | | MDA- | MDA- | U87ΔEGFR- |
| | KPL-4 | 3 | BT-474 | JIMT-1 | MB-453 | MB-231 | luc |
| VCit ADC 4a | 0.084 | 0.119 | 0.498 | 0.101 | 0.183 | — | Not tested |
| | (0.074- | (0.108- | (0.358- | (0.0900- | (0.139- | | |
| | 0.099) | 0.134) | 0.685) | 0.115) | 0.243) | | |
| EVCit ADC 4b | 0.071 | 0.159 | 0.459 | 0.088 | 0.254 | — | Not tested |
| | (0.059- | (0.148- | (0.366- | (0.071- | (0.179- | | |
| | 0.085) | 0.171) | 0.573) | 0.097) | 0.376) | | |
| EGCit ADC 4c | 0.087 | 0.175 | 0.578 | 0.114 | 0.226 | — | Not tested |
| | (0.082- | (0.161- | (0.374- | (0.0960- | (0.170- | | |
| | 0.092) | 0.192) | 0.932) | 0.139) | 0.301) | | |
| EV(N-Me)Cit | 0.217 | 0.167 | 1.145 | 0.373 | 1.004 | — | Not tested |
| 4d | (0.164- | (0.146- | (0.855- | (0.188- | (0.327- | | |
| | 0.284) | 0.192) | 1.480) | 0.895) | 1.186) | | |
| GCit ADC 4e | 0.089 | 0.143 | Not | 0.176 | 0.560 | — | Not tested |
| | (0.084- | (0.107- | tested | (0.154- | (0.440- | | |
| | 0.095) | 0.197) | | 0.199) | 0.737) | | |
| Non-cleavable | 0.544 | Not | Not | 1.758 | Not | Not | Not tested |
| ADC 4f | (0.221- | tested | tested | (Not | tested | tested | |
| | 1.374) | | | determined) | | | |
| Nontargeting | — | — | — | — | — | — | Not tested |
| EGCit ADC 5 | | | | | | | |
| EGCit DuoDM | 0.116 | Not | Not | 0.0593 | Not | Not | Not tested |
| ADC 6 | (0.102- | tested | tested | (0.0384- | tested | tested | |
| | 0.132) | | | 0.0876) | | | |
| EGCit | 0.0159 | Not | Not | 0.0432 | Not | Not | Not tested |
| MMAE/F ADC | (0.0122- | tested | tested | (0.0396- | tested | tested | |
| 7a | 0.0207) | | | 0.0474) | | | |
| VCit MMAE | Not tested | Not | Not | Not tested | Not | Not | 0.344 |
| ADC 8a | | tested | tested | | tested | tested | (0.295- |
| | | | | | | | 0.405) |
| EGCit ADC 8b | Not tested | Not | Not | Not tested | Not | Not | 0.344 |
| | | tested | tested | | tested | tested | (0.296- |
| | | | | | | | 0.401) |
| PABC-DuoDM | Not tested | Not | Not | Not tested | Not | Not | 0.374 |
| ADC S21a | | tested | tested | | tested | tested | (0.339- |
| | | | | | | | 0.410) |
| PABQ-DuoDM | Not tested | Not | Not | Not tested | Not | Not | 0.348 |
| ADC S21b | | tested | tested | | tested | tested | (0.321- |
| | | | | | | | 0.380) |

TABLE 4
Cell viability at the maximum ADC concentration (n =
4). Values in parentheses are 95% confidential intervals.

| | % Cell viability at the maximum ADC concentration | | | | | |
|---|---|---|---|---|---|---|
| | | | | | MDA-MB- | MDA-MB- |
| | KPL-4 | SK-BR-3 | BT-474 | JIMT-1 | 453 | 231 |
| VCit ADC 4a | 4.52 | 33.9 | 21.7 | 16.7 | 34.1 | — |
| | (1.595- | (32.07- | (15.98- | (13.65- | (30.13- | |
| | 7.366) | 35.72) | 26.46) | 19.52) | 37.59) | |
| EVCit ADC 4b | 3.97 | 22.7 | 21.6 | 14.7 | 28.2 | — |
| | (1.076- | (21.46- | (17.75- | (11.46- | (22.32- | |
| | 6.810) | 23.85) | 25.17) | 17.90) | 32.74) | |
| EGCit ADC 4c | 2.70 | 23.0 | 18.1 | 13.5 | 27.4 | — |
| | (1.478- | (21.51- | (8.073- | (9.681- | (22.98- | |
| | 3.922) | 24.38) | 25.95) | 17.06) | 31.61) | |
| EV(N-Me)Cit 4d | 15.5 | 30.4 | 36.2 | 61.1 | 59.0 | — |
| | (10.92- | (28.32- | (32.73- | (53.14- | (6.135- | |
| | 20.30) | 32.41) | 39.61) | 65.96) | 66.90) | |
| GCit ADC 4e | 2.81 | 38.0 | Not tested | 12.9 | 39.4 | — |
| | (1.595- | (33.92- | | (10.72- | (35.53- | |
| | 4.024) | 41.85) | | 15.05) | 42.71) | |
| Non-cleavable | 69.2 | Not tested | Not tested | 87.72 | Not tested | Not tested |
| ADC 4f | (62.81- | | | (Not | | |
| | 74.24) | | | determined) | | |
| EGCit DuoDM | 4.13 | Not tested | Not tested | 17.27 | Not tested | Not tested |
| ADC 6 | (1.373- | | | (11.92- | | |
| | 6.792) | | | 21.79) | | |

TABLE 5
Stability of ADCs in undiluted human plasma. Quantification
was performed after incubation for 7, 14, and 28 days
(n = 3). Data are presented as mean values ± SEM.

| | Residual ADC (%) | | | |
|---|---|---|---|---|
| | Day 0 | Day 7 | Day 14 | Day 28 |
| VCit ADC 4a | 100 ± 13.1 | 96.1 ± 11.0 | 96.6 ± 1.0 | 94.7 ± 13.2 |
| EVCit ADC 4b | 100 ± 2.2 | 92 ± 3.4 | 91.3 ± 1.5 | 95.6 ± 1.3 |
| EGCit ADC 4c | 100 ± 1.7 | 92.2 ± 1.6 | 91.2 ± 2.0 | 90.7 ± 0.2 |

TABLE 6
Stability of ADCs in undiluted monkey plasma. Quantification
was performed after incubation for 7, 14, and 28 days
(n = 3). Data are presented as mean values ± SEM.

| | Residual ADC (%) | | | |
|---|---|---|---|---|
| | Day 0 | Day 7 | Day 14 | Day 28 |
| VCit ADC 4a | 100 ± 19.9 | 87.8 ± 22.4 | 96.0 ± 16.0 | 89.6 ± 11.3 |
| EGCit ADC 4c | 100 ± 18.7 | 92.2 ± 19.3 | 103 ± 21.6 | 107 ± 21.8 |

TABLE 7
Stability of ADCs in undiluted mouse plasma. Quantification
was performed after incubation for 4, 7, and 14 days
(n = 3). Data are presented as mean values ± SEM.

| | Residual ADC (%) | | | |
|---|---|---|---|---|
| | Day 0 | Day 4 | Day 7 | Day 14 |
| VCit ADC 4a | 100 ± 4.7 | 29.0 ± 2.0 | 19.2 ± 1.5 | 15.8 ± 1.7 |
| EVCit ADC 4b | 100 ± 5.3 | 96.0 ± 3.9 | 91.4 ± 9.4 | 95.4 ± 8.7 |
| EGCit ADC 4c | 100 ± 2.5 | 97.6 ± 4.4 | 96.8 ± 3.5 | 98.2 ± 9.0 |

TABLE 8
Statistical significance.

| | | | Crude | Adjusted | |
|---|---|---|---|---|---|
| Figures | Method | Comparison | P value | P value^{a} | Significance |
| FIG. 3J | Dunnett's test | EGCit ADC 4c vs. EV(N- | <0.0001 | N/A | *** |
| | | Me)Cit ADC 4d | 0.3838 | N/A | n.s. |
| | | EGCit ADC 4c vs. VCit | 0.7436 | N/A | n.s. |
| | | ADC 4a | | | |
| | | EGCit ADC 4c vs. EVCit | | | |
| | | ADC 4b | | | |
| FIG. | Dunnett's test | EGCit ADC 4c vs. EVCit | <0.0001 | N/A | *** |
| 4H | | ADC 4b | 0.0324 | N/A | * |
| | | EGCit ADC 4c vs. | | | |
| | | Vehicle | | | |
| FIG. 4I | Dunnett's test | EGCit ADC 4c vs. EVCit | <0.0001 | N/A | *** |
| | | ADC 4b | <0.0001 | N/A | *** |
| | | EGCit ADC 4c vs. | | | |
| | | Vehicle | | | |
| FIG. | Two-tailed | EGCit ADC 4c vs. | 0.0159 | 0.0318 | * |
| 5A | Welch's t-test | Enhertu | 0.0630 | 0.0630 | n.s. |
| | | EGCit ADC 4c vs. | | | |
| | | Kadcyla | | | |
| FIG. | Two-tailed | EGCit ADC 4c vs. | 0.0058 | 0.0116 | * |
| 5B | Welch's t-test | Kadcyla | 0.0414 | 0.0414 | * |
| | | EGCit ADC 4c vs. | | | |
| | | Enhertu | | | |
| FIG. | Two-tailed | EGCit ADC 4c vs. | 0.0789 | 0.1578 | n.s. |
| 5C | Welch's t-test | Kadcyla | 0.2158 | 0.2158 | n.s. |
| | | EGCit ADC 4c vs. | | | |
| | | Enhertu | | | |
| FIG. | Two-tailed | EGCit ADC 4c vs. | 0.0064 | 0.0128 | * |
| 5D | Welch's t-test | Kadcyla | 0.0159 | 0.0159 | * |
| | | EGCit ADC 4c vs. | | | |
| | | Enhertu | | | |
| FIG. | Two-tailed | Dose: 1 mg kg⁻¹ each | 0.0079 | 0.0158 | * |
| 6A | Welch's t-test | EGCit ADC 4c vs. | 0.0097 | 0.0097 | ** |
| | | Enhertu (Day 31) | | | |
| | | EGCit ADC 4c vs. | | | |
| | | Kadcyla (Day 31) | | | |
| FIG. | Logrank | Dose: 1 mg kg⁻¹ each | 0.0019 | 0.0038 | ** |
| 6B | (Mantel-Cox) | EGCit ADC 4c vs. | 0.0021 | 0.0021 | ** |
| | | Enhertu | | | |
| | | EGCit ADC 4c vs. | | | |
| | | Kadcyla | | | |
| FIG. | Two-tailed | 1 mg kg⁻¹ EGCit | 0.0065 | N/A | ** |
| 6C | Welch's t-test | MMAE/F ADC 7a | | | |
| | | vs. 3 mg kg⁻¹ Enhertu | | | |
| | | (Day 31) | | | |
| FIG. | Logrank | Dose: 5 mg kg⁻¹ (each) | 0.0072 | N/A | ** |
| 6D | (Mantel-Cox) | EGCit MMAE/F ADC 7a | | | |
| | | vs. Enhertu | | | |
| FIG. | Logrank | Dose: 5 mg kg⁻¹ (each) | 0.0002 | 0.0006 | *** |
| 6F | (Mantel-Cox) | EGCit ADC 8b vs. VCit | 0.0005 | 0.0010 | *** |
| | | ADC 8a | 0.0010 | 0.0010 | *** |
| | | EGCit ADC 8b vs. | | | |
| | | Vehicle | | | |
| | | VCit ADC 8a vs. Vehicle | | | |

^{a}Adjusted by the Holm-Bonferroni method.
*P < 0.05;
**P < 0.01;
***P < 0.005; n.s., not significant.

In this study, an enzymatically cleavable EGCit, tripeptide linker was demonstrated that can solve the problem of neutropenia, a common clinical off-target toxicity issue originated from human neutrophil protease-mediated degradation of VCit- and similar dipeptide-MMAE ADCs without hindering of the treatment efficacy. The linker cleavage site of human neutrophil elastase was identified using small molecule-based pyrene probes. It was found that P2 modification of VCit and EVCit linker by replacing the valine by glycine could greatly enhance the resistance of neutrophil protease-mediated degradation. The EGCit linker also had greater mouse plasma stability and less hydrophobic compared with VCit and EVCit linker. To show the EGCit-based ADCs have comparable potency with the ADCs containing VCit or similar peptides linkers, EGCit ADCs were constructed using different antibodies and payloads to ensure the broad applicability. In vitro results showed that EGCit ADCs had comparable in vitro cytotoxicity with VCit and EVCit ADCs in various breast and brain cancer lines. The comparable in vitro MMAE release among VCit, EVCit, and EGCit ADCs showed that the EGCit sequence enables efficient traceless payload release upon ADC internalization in a wide range of cell types with varying catabolic profiles. The in vivo treatment efficacy of EGCit ADCs in KPL-4 was also investigated with homogeneous HER2 antigens, admixed JIMT-1/MDA-MB-231 breast with heterogeneous HER2 antigens cancer models, and the aggressive U87ΔEGFR-luc brain cancer model. These three animal models having different antigens and antigen-expressing level ensured the maximal ADC treatment efficacy. Compared with non-cleavable linker having high resistance to proteolytic degradation, the cleavable EGCit linker is more preferable to be used for ADCs. This is because payload catabolites of ADCs containing non-cleavable linker-payload structure after lysosomal degradation of the ADC consist of the linker, a payload, and an amino acid residue derived from the antibody, and thus the linker and payload structures need to be carefully designed not to attenuate their potency. This is why only limited payloads can be chosen for non-cleavable linkers.

### Example 2—Materials and Methods

Antibodies. Anti-HER2, anti-EGFRvIII, and isotype control mAbs with a N297A mutation were expressed in house. The other antibodies used in this study were purchased from commercial vendors as follows: Mouse anti-MMAE/F mAb (LEV-MAF3) from Levena Biopharma; goat anti-human IgG Fab antibody (109-005-097) and goat anti-mouse IgG-HRP conjugate (115-035-071) from Jackson ImmunoResearch; mouse anti-human CD66b FITC conjugate (305104), mouse anti-human CD15 APC conjugate (301908), and mouse anti-human CD34 FITC conjugate (343503) from BioLegend.

Human neutrophil protease-mediated cleavage assay using pyrene probes. Each test compound (10 mM in DMSO, 2 μL) was mixed with 97 μL of tris-buffered saline (TBS, pH 7.4) and 1 μL of 1-pyrenemethylamine (10 mM in DMSO, internal standard). The mixture was incubated at 37° C. for 10 min. Human neutrophil elastase (40 ng μL⁻¹, 20 μL in TBS, MilliporeSigma), human proteinase 3 (250 ng μL⁻¹, 5 μL in TBS, MilliporeSigma) or human cathepsin G (330 ng μL⁻¹, 5 μL in TBS, MilliporeSigma) was added to the test compound mixture in 1:1 volume ratio, followed by incubation at 37° C. Aliquots (5 μL) were collected at each time point (0, 1, 3, 6, and 24 h for elastase; 0 and 24 h for proteinase 3 and cathepsin G). Cold acetonitrile containing 1% formic acid (25 μL) was added to precipitate proteins. The mixture was then kept at −20° C. for 30 min. Precipitated proteins were pelleted by centrifugation (15,000×g, 4° C., 30 min) and supernatant of each sample was analyzed for quantification by analytical HPLC (UV absorbance at 342 nm). The amount of each probe was normalized to the peak area of the internal standard. All assays were performed at least three times in technical duplicate, and data shown are representative of the replicates.

Plasma stability test using pyrene probes. Each test compound (10 mM in DMSO, 2 μL) was mixed with 1 μL of 1-pyrenemethylamine (10 mM in DMSO, internal standard) and incubated at 37° C. for 10 min. Pooled healthy human plasma, monkey cynomolgus plasma or BALB/c mouse plasma (197 μL, Innovative Research) was added to the mixture, followed by incubation at 37° C. Aliquots (5 μL) were collected at each time point (0, 48, and 96 h for human and monkey cynomolgus plasma; 0, 1, 6, 24, 48, and 96 h for BALB/c mouse plasma) and 25 μL of cold acetonitrile containing 1% formic acid was added to precipitate proteins. The mixture was then kept at −20° C. for 30 min. Precipitated proteins were separated by centrifugation (15,000×g, 4° C., 30 min) and supernatant of each sample was obtained and analyzed for quantification by analytical HPLC as described above. All assays were performed at least three times in technical duplicate, and data shown are representative of the replicates.

MTGase-mediated antibody-linker conjugation. Anti-HER2 mAb with a N297A mutation (1.06 mL in PBS, 10.5 mg mL⁻¹, 11.1 mg antibody) was incubated with the diazide branched linker developed previously (Anami et al., 2018; Anami et al., 2020; Anami et al., 2017) (37.1 μL of 100 mM stock in water, 50 equiv.) and Activa TI® (275 μL of 40% solution in PBS, Ajinomoto, purchased from Modernist Pantry) at room temperature for 16-20 h. The reaction was monitored using a Thermo LC-MS system consisting of a Vanquish UHPLC and a Q Exactive™ Hybrid Quadrupole-Orbitrap™ Mass Spectrometer equipped with a MabPac RP column (2.1×50 mm, 4 μm, Thermo Scientific). Elution conditions were as follows: mobile phase A=water (0.1% formic acid); mobile phase B=acetonitrile (0.1% formic acid); gradient over 3 min from A:B=75:25 to 45:55; flow rate=0.25 mL min⁻¹. The conjugated antibody was purified by SEC (Superdex 200 increase 10/300 GL, GE Healthcare, solvent: PBS, flow rate=0.6 mL min⁻¹) to afford an antibody-linker conjugate [9.62 mg, 86% yield determined by bicinchoninic acid (BCA) assay]. The other antibody-linker conjugates used in this study were prepared in the same manner.

Strain-promoted azide-alkyne cycloaddition for payload installation. BCN-PEG₃-EGCit-PABC-MMAE (18.9 μL of 10 mM stock solution in DMSO, 2 equivalent per azide group) was added to a solution of the mAb-linker conjugate in PBS (1.70 mL, 2.1 mg mL⁻¹), and the mixture was incubated at room temperature for 17 h. The reaction was monitored using a Thermo LC-MS system consisting of a Vanquish UHPLC and a Q Exactive™ Hybrid Quadrupole-Orbitrap™ Mass Spectrometer equipped with a MabPac RP column. The crude products were purified by SEC to afford an ADC [3.0 mg, 85% yield determined by bicinchoninic acid (BCA) assay]. Analysis and purification conditions were the same as described above. Average DAR values were determined based on UV peak areas and ESI-MS analysis. The other conjugates used in this study were prepared in a similar manner or according to previous reports (Anami et al., 2018; Anami et al., 2020; Anami et al., 2017; Yamazaki et al., 2021; Yamaguchi et al., 2021; Anami et al., 2020).

HIC analysis. Each ADC (1 mg mL⁻¹, 10 μL in PBS) was analyzed using an Agilent 1100 HPLC system equipped with a MAbPac HIC-Butyl column (4.6×100 mm, 5 μm, Thermo Scientific). Elution conditions were as follows: mobile phase A=50 mM sodium phosphate containing ammonium sulfate (1.5 M) and 5% isopropanol (pH 7.4); mobile phase B=50 mM sodium phosphate containing 20% isopropanol (pH 7.4); gradient over 30 min from A:B=99:1 to 1:99; flow rate=0.5 mL min⁻¹.

Long-term stability test. Each ADC (1 mg mL⁻¹, 100 μL in PBS) was incubated at 37° C. Aliquots (8 μL) were taken at 28 days and immediately stored at −80° C. until use. Samples were analyzed using an Agilent 1100 HPLC system equipped with a MAbPac SEC analytical column (4.0×300 mm, 5 μm, Thermo Scientific). Elution conditions were as follows: flow rate=0.2 mL min⁻¹; solvent=PBS.

Cell culture. U87ΔEGFR-luc was generated by lentiviral transduction of U87ΔEGFR cells (a gift from Dr. Balveen Kaur, UTHealth) using LENTIFECT™ lentiviral particles encoding for firefly luciferase and a puromycin-resistant gene (GeneCopoeia, LP461-025). Transduction was performed according to the manufacturer's instruction. JIMT-1 (AddexBio), SK-BR-3 (ATCC), and BT-474 (ATCC) were cultured in RPMI1640 (Corning) supplemented with 10% EQUAFETAL® (Atlas Biologicals), GLUTAMAX® (2 mM, Gibco), sodium pyruvate (1 mM, Corning), and penicillin-streptomycin (penicillin: 100 units mL⁻¹; streptomycin: 100 μg mL⁻¹, Gibco). KPL-4 (provided by Dr. Junichi Kurebayashi at Kawasaki Medical School), MDA-MB-453 (ATCC), MDA-MB-231 (ATCC), and U87ΔEGFR-luc were cultured in DMEM (Corning) supplemented with 10% EQUAFETAL®, GLUTAMAX® (2 mM), and penicillin-streptomycin (penicillin: 100 units mL⁻¹; streptomycin: 100 μg mL⁻¹). All cells were cultured at 37° C. under 5% CO₂ and passaged before becoming fully confluent up to 20 passages. All cell lines were periodically tested for mycoplasma contamination.

Cell viability assay. Cells were seeded in a culture-treated 96-well clear plate (5,000 cells per well in 50 μL culture medium) and incubated at 37° C. under 5% CO₂ for 24 h. Serially diluted samples (50 μL) were added to each well and the plate was incubated at 37° C. for 72 h for KPL-4, SK-BR-3, MDA-MB-231, and U87ΔEGFR-luc cells, and 96 h for JIMT-1, BT-474, and MDA-MB-453 cells. For DuoDM-ADCs, all the tested cell lines were incubated for 120 h. After the old medium was replaced with 100 μL fresh medium, 20 μL of a mixture of WST-8 (1.5 mg mL⁻¹, Cayman chemical) and 1-methoxy-5-methylphenazinium methylsulfate (1-methoxy PMS, 100 μM, Cayman Chemical) was added to each well, and the plate was incubated at 37° C. for 2 h. After gently agitating the plate, the absorbance at 460 nm was recorded using a BioTek Synergy HTX plate reader. EC₅₀ values were calculated using Graph Pad Prism 8 software. All assays were performed in triplicate (in U87ΔEGFR-luc cells line) or quadruplicate (other cell lines).

Determination of concentration of cellular MMAE. KPL-4 cells were seeded in a culture-treated 12-well plate (3×10⁵ cells per well in 500 μL culture medium) and incubated at 37° C. under 5% CO₂ for 24 h. Each ADC (final conc. 4 μg mL⁻¹, 26.7 nM) was added to each well and the plate was incubated at 37° C. for 24 h. After the plate was centrifuged (5 min at 500×g at 4° C.), the supernatant (450 μL) was collected then proteins were precipitated by adding MeOH (600 μL). Cells were lysed with MeOH (500 μL) and transferred to a microtube. Additional 100 μL of MeOH was added to rinse the plate and transferred to the microtube. The supernatant and cell lysate were centrifuged (10 min at 15,000×g), and each supernatant was transferred to microtubes. These supernatants were then dried by air flow at 40° C. After drying over, MS grade water (30 μL) was added, and the mixture was centrifuged (5 min at 15,000×g). Then the supernatant was analyzed by the Q EXACTIVE™ HYBRID QUADRUPOLE-ORBITRAP™ Mass Spectrometer. For quantitation, six-point standard curves were made using serial dilutions of free MMAE (0.032-100 nM). Non-treated cell lysates were used for the free MMAE standard curves. All assays were performed in triplicate.

Plasma stability test using ADCs. [1] Stability in mouse plasma. Each ADC (100 μg mL⁻¹, 1.2 μL in PBS) was added to undiluted BALB/c mouse plasma (118.8 μL) to a final concentration of 1 μg mL⁻¹. After incubation at 37° C. for varying time, aliquots (15 μL each) were taken and stored at −80° C. until use. Samples were analyzed by sandwich ELISA assay. A high-binding 96 well plate (Corning) was coated with goat anti-human IgG Fab antibody (500 ng per well). After overnight coating at 4° C., the plate was blocked with 100 μL of 2% BSA in PBS containing 0.05% Tween 20 (PBS-T) with agitation at room temperature for 1 h. Subsequently, the solution was removed and each ADC sample (100 μL in PBS-T containing 1% BSA) was added to each well, and the plate was incubated at room temperature for 2 h. After each well was washed three times with 100 μL of PBS-T, 100 μL of mouse anti-MMAE/F mAb (1:5,000) was added. After being incubated at room temperature for 1 h, each well was washed three times with 100 μL of PBS-T, 100 μL of goat anti-mouse IgG-HRP conjugate (1:10,000) was added. After 1 h, the plate was washed three times with 100 μL of PBS-T and 100 μL of 3,3′,5,5′-tetramethylbenzidine (TMB) substrate (0.1 mg mL⁻¹) in phosphate-citrate buffer/30% H₂O₂ (1:0.0003 volume to volume, pH 5) was added. After color was developed for 10-30 min, 25 μL of 3N-HCl was added to each well and then the absorbance at 450 nm was recorded using a plate reader (Biotek Cytation 5). Concentrations were calculated based on a standard curve.

[2] Stability in human plasma and monkey plasma. Assays were performed in the same manner using human HER2 (100 ng per well, ACROBiosystems) for plate coating, mouse anti-MMAE/F mAb (1:5,000) and goat anti-mouse IgG-HRP conjugate (1:10,000) as secondary and tertiary detection antibodies, respectively. All assays were performed in triplicate.

Human neutrophil enzymes-mediated cleavage assay using ADCs. Each ADC (1 mg mL⁻¹, 30 μL) in TBS buffer was incubated at 37° C. for 10 min. To the solution was added pre-warmed human neutrophil elastase (1 ng μL⁻¹, 30 μL, MilliporeSigma), followed by incubation at 37° C. for 24 h. The samples were analyzed using a Thermo LC-MS system consisting of a Vanquish UHPLC and a Q Exactive™ Hybrid Quadrupole-Orbitrap™ Mass Spectrometer equipped with a MabPac RP column (2.1×50 mm, 4 μm, Thermo Scientific). Elution conditions were as follows: mobile phase A=water (0.1% formic acid); mobile phase B=acetonitrile (0.1% formic acid); gradient over 3 min from A:B=75:25 to 45:55; flow rate=0.25 mL min⁻¹. Note: Hinge cleavage of the anti-HER2 mAb and ADCs was observed in the analysis.

Human neutrophil killing assay. This assay was performed following the protocol reported by Zhao et al. (Zhao et al., 2018) with modifications. CD34-positive HSPCs isolated from the bone marrow were purchased from AllCells (received as a cryopreserved sample and stored in liquid nitrogen until use). Reagents purchased from StemCell Technologies for this assay are as follows: cell culture medium (STEMSPAN™ SFEM II, 09655), supplements for expansion (STEMSPAN™ CC100, 02690), interleukin 3 (IL-3), stem cell factors (SCF), Flt-3 ligands (Flt-3L), granulocyte-macrophage colony-stimulating factors (GM-CSF), and granulocyte colony-stimulating factors (G-CSF). HSCs (2×10⁴ cells mL⁻¹) were expanded in STEMSPAN™ SFEM II supplemented with CC100 for 3 days. Prior to differentiation, the expression levels of granulocyte markers (CD66b and CD15) were measured using a BD LSR II flow cytometer (Day 0, see the next section for details). Expanded HSCs were incubated for 4 days in STEMSPAN™ SFEM II supplemented with SCF (50 ng mL⁻¹), Flt-3L (100 ng mL⁻¹), IL-3 (5 ng mL⁻¹), GM-CSF (5 ng mL⁻¹), and G-CSF (5 ng mL⁻¹). On Day 4, cell culture medium was replaced with STEMSPAN™ SFEM II supplemented with IL-3 (5 ng mL⁻¹) and G-CSF (30 ng mL⁻¹) and cells were further incubated for 3 days. On Day 7, differentiated cells were measured for CD15 and CD66b by flow cytometry. Cell culture medium was replaced with STEMSPAN™ SFEM II supplemented with G-CSF (30 ng mL⁻¹), 1% penicillin-streptomycin, and vehicle control or each ADC (200 nM) and cell density was adjusted to 1×10⁵ cells mL⁻¹. After being incubated for 7 days (Day 14), cells were measured for CD15 and CD66b by flow cytometry. Effect of each ADC on the population of neutrophils was represented by percentage of CD66b/CD15-double positive cells in the viable cell population.

Flow cytometry. Cells were washed twice with cold Stain Buffer (1 mL for microtubes; 200 μL for microwell plates, FBS, BD Bioscience, catalog #: 554656) and harvested by centrifugation (400×g, 5 min at 4° C.). Then, the cells were resuspended with cold Stain Buffer (100 μL) in microtubes or microwell plates and mixed with 5 μL of fluorescently labeled antibodies (mouse anti-human CD66b FITC conjugate, mouse anti-human CD15 APC conjugate, or mouse anti-human CD34 FITC conjugate). To set voltages and gating parameters for obtaining accurate fluorescence signal, a drop of ULTRACOMP EBEADS™ Compensation Beads (Invitrogen, catalog #: 01-2222-42) was also labeled with 5 μL of the antibodies separately from the cells. Cells and beads were incubated in the dark on ice for 20 min, then washed three times with either 1 mL (for microtubes) or 200 μL (for microwell plates) of cold Stain Buffer to remove unbound antibodies. After centrifugation (400×g, 5 min at 4° C.), the supernatants were carefully aspirated from cell pellets or beads. Stained cells or beads were resuspended with 1 mL of cold Stain Buffer, transferred to tubes for flow cytometric analysis, and data were acquired with LSR II flow cytometer with Diva acquisition software (BD Biosciences) gated for live cells with appropriate isotype-matched controls and unstained cells as negative controls. Percentage of CD66b and CD15 generated from flow cytometry was normalized against non-treated samples. All assays were performed in triplicate.

Animal studies. All procedures were approved by the Animal Welfare Committee of the University of Texas Health Science Center at Houston and performed in accordance with the institutional guidelines for animal care and use. All animals were housed under controlled conditions, namely 21-22° C. (±0.5° C.), 30-75% (±10%) relative humidity, and 12:12 light/dark cycle with lights on at 7.00 am. Food and water were available ad libitum for all animals.

Tolerability study. Female 6-8 weeks old CD-1® IGS mice (6-8 weeks old, Charles River Laboratories, Strain Code: 022) received a single dose of each ADC (80 mg kg⁻¹) intraperitoneally. Body weight was monitored every day for 5 days. Humane endpoints were defined as 1) greater than 20% weight loss or 2) severe signs of distress. However, no mice met these criteria over the course of study. Five days post injection, these mice were deeply anesthetized with isoflurane and the whole blood was drawn by heart puncture for following hematology and blood chemistry analysis (Vehicle, n=4; KADCYLA®, n=6; ENHERTU®, n=7; EGCit ADC 4c, n=10).

Blood chemistry and hematology analysis. Blood chemistry: whole blood (400-600 μL) was drawn using S-MONOVETTE® charged with or serum gel (1.1 mL syringe, Sarstedt) and allowed to clot at room temperature for 30-40 min. After centrifugation at 2,000×g for 10 min, resulting serum samples (150 μL) were loaded onto NSAID 6 clips specialized for identifying liver damage (IDEXX, Westbrook, ME) and analyzed using a Catalyst Dx Chemistry Analyzer (IDEXX).

Hematology. Whole blood (700-1,000 μL) was drawn using S-MONOVETTE® charged with K₃ EDTA (1.1 mL syringe, Sarstedt). Blood samples were gently mixed well by inversion and stored on ice until analysis (for less than 4 h). Each blood sample (500 μL) was analyzed using a PROCYTE DX® (IDEXX).

In vivo xenograft mouse models of human breast cancer. KPL-4 model: cells (1×10⁷ cells) suspended in 100 μL of 1:1 PBS/CULTREX® BME Type 3 (Trevigen) were orthotopically injected into the inguinal mammary fat pad of female NSG mice (6-8 weeks old, purchased from The Jackson Laboratory, Stock number: 005557, maintained by in-house breeding). When the tumor volume reached ˜100 mm³, mice were randomly assigned to six groups (n=5 for each group) and a single dose of each ADC (1 mg kg⁻¹) or vehicle was administered to mice intravenously. Tumor volume (0.52×a×b², a: long diameter, b: short diameter) and body weight were monitored twice a week using a digital caliper. Mice were euthanized when the tumor volume exceeded 1,000 mm³, the tumor size exceeded 2 cm in diameter, greater than 20% weight loss was observed, or mice showed signs of distress. Such events were counted as deaths.

JIMT-1/MDA-MB-231 admixed tumor model. A co-suspension of 1×10⁷ JIMT-1 cells and 2.5×10⁶ MDA-MB-231 cells in 100 μL of 1:1 PBS/CULTREX® BME Type 3 (Trevigen) was orthotopically injected into the inguinal mammary fat pad of female NU/J mice (6-8 weeks old, The Jackson Laboratory, Stock number: 002019). On day 7 post transplantation, mice were randomly assigned to each group (n=5 for ENHERTU®; n=6 for EGCit-MMAE/F dual-drug ADC 7a) and injected intravenously with sterile-filtered human IgG (30 mg kg⁻¹, Innovative Research, catalog number: IRHUGGFLY1G) in PBS. The next day, a single dose of ENHERTU® (3 mg kg⁻¹) or dual-drug ADC 7a (1 mg kg⁻¹) was administered to mice intravenously. Tumor volume (0.52×a×b², a: long diameter, b: short diameter) and body weight were monitored twice a week. Mice were euthanized when the tumor volume exceeded 1,000 mm³, the tumor size exceeded 2 cm in diameter, or mice showed severe signs of distress. Such events were counted as deaths.

Orthotopic xenograft mouse model of human GBM. U87ΔEGFR-luc cells (1×10⁵ cells) were stereotactically implanted into NSG mice (6-8 weeks old, male and female) as follows. NSG mice were injected intraperitoneally with a cocktail of ketamine (67.5 mg kg⁻¹) and dexmedetomidine (0.45 mg kg⁻¹) and maintained at 37° C. on a heating pad until the completion of surgery. After the head skin was shaved and treated with 10 μL of 0.25% bupivacaine supplemented with epinephrine (1:200,000), anesthetized mice were placed on a stereotactic instrument. After disinfecting the head skin with chlorhexidine and ethanol, a small incision was made and then a burr hose was drilled into the skull over the right hemisphere (1 mm anterior and 2 mm lateral to the bregma). A 10 μL Hamilton syringe (model 701 N) was loaded with cells suspended in 2 μL cold hanks-balanced salt solution (HBSS) and slowly inserted into the right hemisphere through the burr hole (3.5 mm depth). After a 1-min hold time, cells were injected over a 5-min period (0.4 μL min⁻¹). After a 3-min hold time, the needle was retracted at a rate of 0.75 mm min⁻¹. The incision was closed using GLUTURE® (Zoetis) and mice were injected with atipamezole (1 mg kg⁻¹, i.p.). At day 5 post implantation, brain tumor-bearing NSG mice were randomized and injected intravenously with a single dose of either ADC (5 mg kg⁻¹, n=6 for VCit ADC 8a; n=7 for EGCit ADC 8b) or PBS (n=6). Body weight was monitored every 3-4 days and mice were euthanized when body weight loss of >20% or any severe clinical symptom was observed. Such events were counted as deaths.

Data Reporting. Although no statistical analysis was performed prior to performing experiments, sample size was determined by following methods for similar experiments in the field reported previously. We did not use the vehicle control in the xenograft breast cancer studies for statistical analysis. The investigators were not blinded to allocation during experiments. For determination of cellular MMAE concentration and human neutrophil killing, a one-way ANOVA with a Dunnett's post hoc test was used for multiple comparisons. For blood chemistry and hematology analysis and xenograft tumor model studies, a Welch's t-test (two-tailed, unpaired, uneven variance) was used. Kaplan-Meier survival curve statistics were analyzed with a logrank (Mantel-Cox) test. To control the family-wise error rate in multiple comparisons, crude P values were adjusted by the Holm-Bonferroni method. Differences with adjusted P values less than 0.05 were considered statistically significant in all analysis. See Table 8 for all P values.

Data Availability. All data supporting the findings in this study are available within the paper, its supplementary information file, or from the corresponding author upon reasonable request.

### Example 3—Synthesis

Unless otherwise noted, all materials for chemical synthesis were purchased from commercial suppliers (Acros Organics, AnaSpec, Broadpharm, Chem-Impex International, Fisher Scientific, Levena Biopharma, Sigma Aldrich, TCI America, and other vendors) and used as received. All anhydrous solvents were purchased and stored over activated molecular sieves under argon atmosphere.

Analytical reverse-phase high performance liquid chromatography (RP-HPLC) was performed using a Thermo LC-MS system consisting of a Vanquish UHPLC and a LTQ XL™ linear ion trap mass spectrometer equipped with a C18 reverse-phase column (ACCUCORE™ VANQUISH™ C18+ UHPLC column, 2.1×50 mm, 1.5 μm, Thermo Scientific). Standard analysis conditions for organic molecules were as follows: flow rate=0.5 mL min⁻¹; solvent A=water containing 0.1% formic acid; solvent B=acetonitrile containing 0.1% formic acid. Compounds were analyzed using a linear gradient and monitored with UV detection at 210 and 254 nm. Preparative HPLC was performed using a Breeze HPLC system (Waters) equipped with a C18 reverse-phase column (XBridge Peptide BEH C18 OBD Prep Column, 130 Å, 5 μm, 19×150 mm, Waters). Standard purification conditions were as follows: flow rate=20 mL min⁻¹; solvent A=water containing 0.05% trifluoroacetic acid (TFA), 0.1% formic acid or 0.1% NH₄OH; solvent B=acetonitrile containing 0.05% TFA (standard conditions), 0.1% formic acid (FA conditions), or 0.1% NH₄OH (basic conditions). Compounds were analyzed using a linear gradient and monitored with UV detection at 210 and 254 nm. In all cases, fractions were analyzed off-line using either of the LC-MS systems for purity confirmation and those containing a desired product were lyophilized using a Labconco Freezone 4.5 Liter Benchtop Freeze Dry System. High-resolution mass spectra were obtained using a THERMO Q EXACTIVE™ HYBRID QUADRUPOLE-ORBITRAP™ Mass Spectrometer.

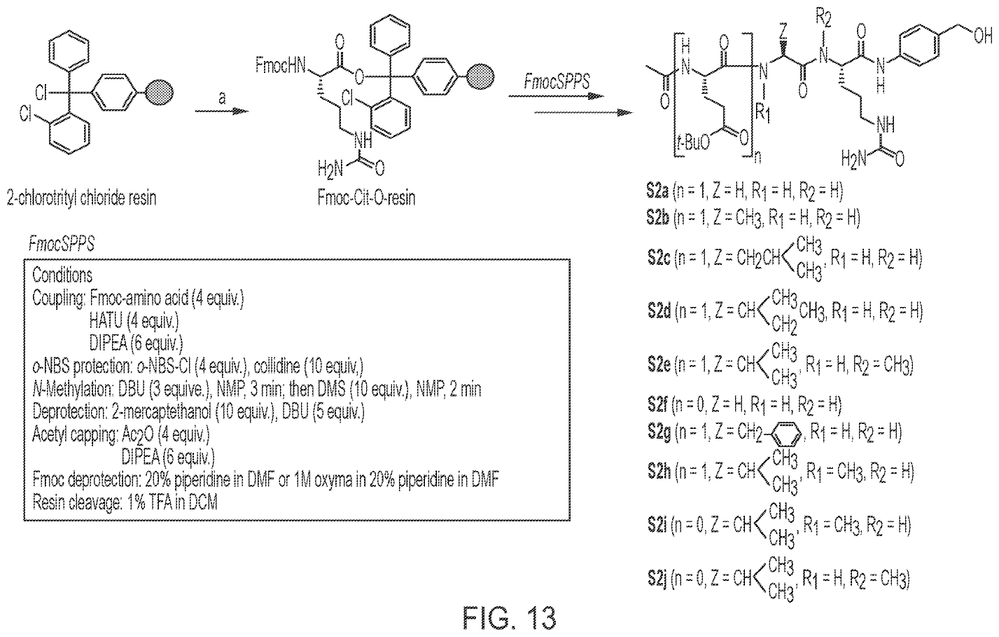

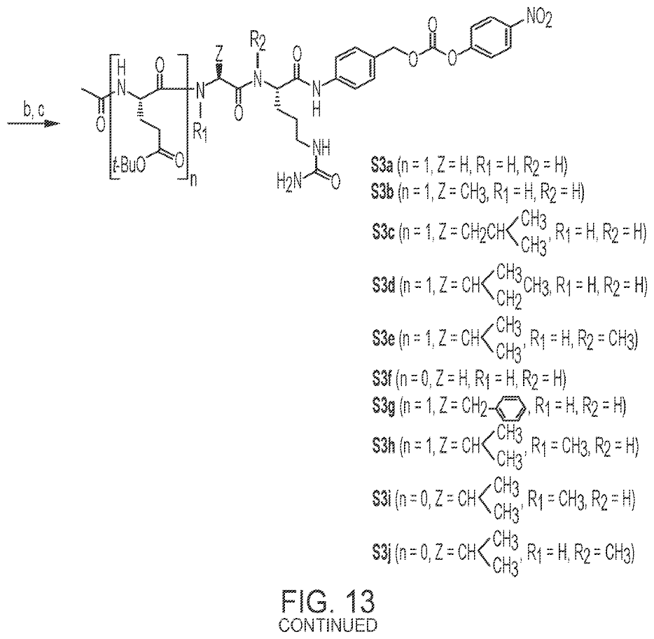

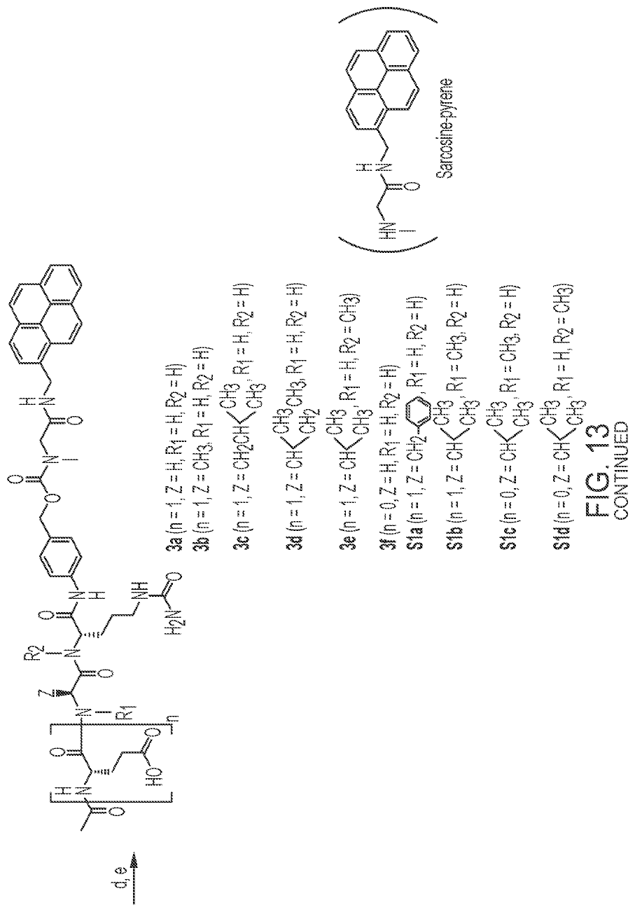

Fmoc Solid-Phase Peptide Synthesis (Fmoc SPPS) for acetyl-capped compounds (S2a-j) 2-Chlorotrityl chloride resin (600 mg, 0.96 mmol) and Fmoc-citrulline-OH (1.8 equiv.) were taken to a manual solid-phase reactor containing N,N-diisopropylethylamine (DIPEA, 3 equiv.) and DMF (3 mL) and agitated for 2 h. MeOH (600 μL) was added to the resin and agitated for 20 min. The solution was drained and the resin was washed with DMF (5×3 mL) and DCM (5×3 mL). To remove a Fmoc-protecting group after each coupling, the resin was treated with 20% piperidine/DMF (5 mL) or 1M oxyma in 20% piperidine/DMF (5 mL) for 20 min and washed with DMF (5×3 mL) and DCM (5×3 mL). For N-methylation¹, the resin was treated with 2-nitrobenzenesulfonyl chloride (o-NBS-Cl, 4 equiv.) and collidine (10 equiv.) in NMP (2 mL) for 15 min and washed with NMP (2×1 mL) to temporally protect amine group with o-NBS. After repeating o-NBS protection twice, the resin was treated with 1,8-diazabicyclo[5.4.0]undec-7-ene (DBU, 3 equiv.) in NMP (1 mL) for 3 min then treated with dimethyl sulfate (DMS, 10 equiv.) in NMP (1 mL) for 2 min. This step was repeated twice. Then, the resin was treated with 2-mercaptethanol (10 equiv.) and DBU (5 equiv.) in NMP (2 mL) for 5 min twice to deprotect o-NBS protecting group. Fmoc-protected amino acid (4 equiv.) was pre-activated using 1-[bis(dimethylamino)methylene]-1H-1,2,3-triazolo[4,5-b]pyridinium 3-oxid hexafluorophosphate (HATU, 4 equiv.) and DIPEA (6 equiv.) in DMF for 3 min, and the cocktail was added to the resin. The resin was agitated for 1 h at room temperature. The completion of the coupling was verified by the Kaiser test. After each coupling step, the coupling cocktail was drained and the resin was washed with DMF (5×3 mL) and DCM (5×3 mL). After elongation of the peptide, the resin was treated with acetic anhydride (4 equiv.) and DIPEA (6 equiv.) in DMF for 1 h and then washed with DMF (5×3 mL) and DCM (5×3 mL). The resulting protected peptide resin was treated with cocktail of 1% trifluoroacetic acid (TFA)/DCM at room temperature for 1 h. The solution was concentrated in vacuo and the crude peptide was precipitated with cold diethyl ether (20 mL) followed by centrifugation at 2,000×g for 3 min (3 times). The resulting crude peptide S2a-j was dried in vacuo and then used immediately in the next step without purification (FIG. 13).

Ac-Glu(t-Bu)-Gly-Cit-PABC-PNP (S3a). To a solution of crude S2a (9.5 mg, 21 μmol) in DCM/MeOH (4:1, 1.25 mL) were added p-aminobenzyl alcohol (5.2 mg, 42 μmol) and EEDQ (10.4 mg, 42 μmol). After being stirred in the dark at room temperature overnight, the solution was concentrated in vacuo and the crude peptide was precipitated with cold diethyl ether (20 mL) followed by centrifugation at 2,000×g for 3 min (10 times). The resulting crude peptide was dried in vacuo and then used immediately in the next step without purification. Bis(2,4-dinitrophenyl) carbonate (27.4 mg, 90 μmol) and DMAP (4.4 mg, 36 μmol) were added to a solution of the crude peptide (10.2 mg, 18 μmol) in DMF (300 μL), and the mixture was stirred at room temperature for 2 h. The reaction was quenched with 3-N HCl/ACN at 0° C., then the crude products were purified by preparative RP-HPLC to afford analytically pure peptide S3a (6.0 mg, 39% for the 2 steps). Purity was confirmed by LC-MS. White powder. HRMS (ESI) Calcd. For C₃₃H₄₄N₇O₁₂ [M+H]⁺: 730.3043. Found: 730.3033. Peptides S3b-j were synthesized from S2b-j in a similar manner.

Ac-Glu(t-Bu)-Ala-Cit-PABC-PNP (S3b). 47.9 mg, 80% for the 2 steps. White powder. HRMS (ESI) Calcd. For C₃₄H₄₆N₇O₁₂ [M+H]⁺: 744.3199. Found: 744.3177.

Ac-Glu(t-Bu)-Leu-Cit-PABC-PNP (S3c). 8.1 mg, 29% for the 2 steps. White powder. HRMS (ESI) Calcd. For C₃₇H₅₂N₇O₁₂ [M+H]⁺: 786.3669. Found: 786.3647.

Ac-Glu(t-Bu)-Ile-Cit-PABC-PNP (S3d). 42 mg, 66% for the 2 steps. White powder. HRMS (ESI) Calcd. For C₃₇H₅₂N₇O₁₂ [M+H]⁺: 786.3669. Found: 786.3644.

Ac-Glu(t-Bu)-Val-(N-Me)Cit-PABC-PNP (S3e). 6.4 mg, 21% for the 2 steps. White powder. HRMS (ESI) Calcd. For C₃₇H₅₂N₇O₁₂ [M+H]⁺: 786.3669. Found: 786.3643.

Ac-Gly-Cit-PABC-PNP (S3f). 14.2 mg, 31% for the 2 steps. White powder. HRMS (ESI) Calcd. For C₂₄H₂₉N₆O₉ [M+H]⁺: 545.1991. Found: 545.1965.

Ac-Glu(t-Bu)-Phe-Cit-PABC-PNP (S3g). 14.7 mg, 44% for the 2 steps. White powder. HRMS (ESI) Calcd. For C₄₀H₅₀N₇O₁₂ [M+H]⁺: 820.3512. Found: 820.3495.

Ac-Glu(t-Bu)-(N-Me)Val-Cit-PABC-PNP (S3h). 14.1 mg, 57% for the 2 steps. White powder. HRMS (ESI) Calcd. For C₃₇H₅₂N₇O₁₂ [M+H]⁺: 786.3669. Found: 786.3642.

Ac—(N-Me)Val-Cit-PABC-PNP (S3i). 9.2 mg, 19% for the 2 steps. White powder. HRMS (ESI) Calcd. For C₂₈H₃₇N₆O₉ [M+H]⁺: 601.2617. Found: 601.2591.

Ac-Val-(N-Me)Cit-PABC-PNP (S3j). 6.1 mg, 20% for the 2 steps. White powder. HRMS (ESI) Calcd. For C₂₈H₃₇N₆O₉ [M+H]⁺: 601.2617. Found: 601.2602.

Ac-Glu-Gly-Cit-PABC-sar-pyrene (3a). Compound S3a (6.0 mg, 8.2 μmol) was dissolved in 20% TFA/DCM (200 μL and 800 μL). After being stirred at room temperature for 40 min, the solution was concentrated in vacuo and the crude peptide was precipitated with cold diethyl ether (20 mL) followed by centrifugation at 2,000×g for 3 min (3 times). The resulting crude peptide was dried in vacuo and then used immediately in the next step without purification. The crude peptide was dissolved in DMF (300 μL) and to the solution were added sarcosine-pyrene² (54.5 μL, 9.0 μmol, 50 mg mL⁻¹ in DMSO), DIPEA (2.9 μL, 16.4 μmol), and DMAP (10 μL, 10 μmol %, 10 mg mL⁻¹ in DMF). The mixture was stirred at 37° C. for 4 h and purified by preparative RP-HPLC under acidic conditions to afford 3a (3.3 mg, 48% for the 2 steps). White powder. HRMS (ESI) Calcd. For C₄₃H₄₉N₈O₁₀ [M+H]⁺: 837.3566. Found: 837.3525. Peptides 3b-f and S1a-d were synthesized from S3b-j in a similar manner.

Ac-Glu-Ala-Cit-PABC-sar-pyrene (3b). 8.1 mg, 35% for the 2 steps. White powder. HRMS (ESI) Calcd. For C₄₄H₅₁N₈O₁₀ [M+H]⁺: 851.3723. Found: 851.3683.

Ac-Glu-Leu-Cit-PABC-sar-pyrene (3c). 5.0 mg, 59% for the 2 steps. White powder. HRMS (ESI) Calcd. For C₄₇H₅₇N₈O₁₀ [M+H]⁺: 893.4192. Found: 893.4163.

Ac-Glu-Ile-Cit-PABC-sar-pyrene (3d). 0.8 mg, 3.5% for the 2 steps. White powder. HRMS (ESI) Calcd. For C₄₇H₅₇N₈O₁₀ [M+Na]⁺: 893.4192. Found: 893.4178.

Ac-Glu-Val-(N-Me)Cit-PABC-sar-pyrene (3e). 0.4 mg, 5.5% for the 2 steps. White powder. HRMS (ESI) Calcd. For C₄₇H₅₇N₈O₁₀ [M+H]⁺: 893.4192. Found: 893.4183.

Ac-Gly-Cit-PABC-sar-pyrene (3f). 4.2 mg, 74%. White powder. HRMS (ESI) Calcd. For C₃₈H₄₂N₇O₇ [M+H]⁺: 708.3140. Found: 708.3128.

Ac-Glu-Phe-Cit-PABC-sar-pyrene (S1a). 6.9 mg, 58% for the 2 steps. White powder. HRMS (ESI) Calcd. For C₅₀H₅₅N₈O₁₀ [M+H]⁺: 927.4036. Found: 927.4027.

Ac-Glu-(N-Me)Val-Cit-PABC-sar-pyrene (S1b). 3.1 mg, 45% for the 2 steps. White powder. HRMS (ESI) Calcd. For C₄₇H₅₇N₈O₁₀ [M+H]⁺: 893.4192. Found: 893.4177.

Ac—(N-Me)Val-Cit-PABC-sar-pyrene (S1c). 9.0 mg, 79%. White powder. HRMS (ESI) Calcd. For C₄₂H₅₀N₇O₇[M+H]⁺: 764.3766. Found: 764.3738.

Ac-Val-(N-Me)Cit-PABC-sar-pyrene (S1d). 2.5 mg, 33%. White powder. HRMS (ESI) Calcd. For C₄₂H₅₀N₇O₇ [M+H]⁺: 764.3766. Found: 764.3750.

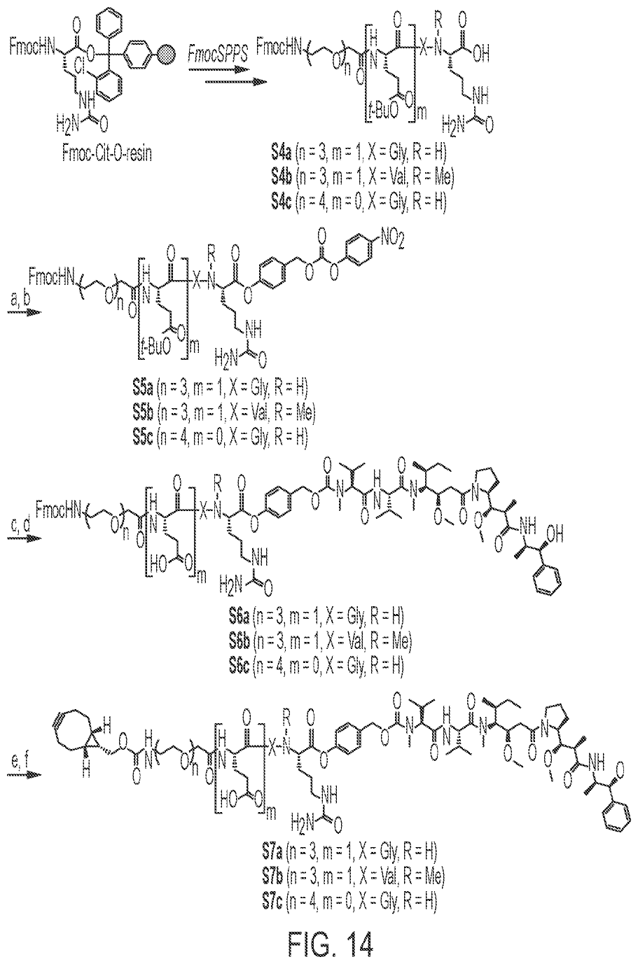

Fmoc Solid-Phase Peptide Synthesis (Fmoc SPPS) for Fmoc-protected compounds (S4a-c). Fmoc-protected peptide compounds S4a-c were prepared as described above. The resulting protected peptide resin was treated with cocktail of 1% trifluoroacetic acid (TFA)/DCM at room temperature for 1 h. The solution was concentrated in vacuo and the crude peptide was precipitated with cold diethyl ether (20 mL) followed by centrifugation at 2,000×g for 3 min (3 times). The resulting crude peptide S4a-c was dried in vacuo and then used immediately in the next step without purification (FIG. 14).

Fmoc-peg3-Glu(t-Bu)-Gly-Cit-PABC-PNP (S5a). To a solution of crude S4a (158.6 mg, 192 μmol) in DCM/MeOH (4:1, 2.5 mL) were added p-aminobenzyl alcohol (70.8 mg, 576 μmol) and EEDQ (285 mg, 1.15 mmol). After being stirred in the dark at room temperature overnight, the solution was concentrated in vacuo and the crude peptide was precipitated with cold diethyl ether (20 mL) followed by centrifugation at 2,000×g for 3 min (10 times). The resulting crude peptide was dried in vacuo and then used immediately in the next step without purification.

Bis(2,4-dinitrophenyl) carbonate (55 mg, 183 μmol) and DMAP (8.9 mg, 73 μmol) were added to a solution of the crude peptide (34.1 mg, 36.5 μmol) in DMF (1 mL), and the mixture was stirred at room temperature for 2 h. The reaction was quenched with 3-N HCl/ACN at 0° C., then the crude products were purified by preparative RP-HPLC to afford analytically pure peptide S5a (25.1 mg, 63% for the 2 steps). Purity was confirmed by LC-MS. White powder. HRMS (ESI) Calcd. For C₅₄H₆₇N₈O₁₇ [M+H]⁺: 1099.4619. Found: 1099.4602. Peptides S5b and S5c were synthesized from S4b and S4c in a similar manner.

Fmoc-peg3-Glu(t-Bu)-Val-(N-Me)Cit-PABC-PNP (S5b). 8.2 mg, 17% for the 2 steps. White powder. HRMS (ESI) Calcd. For C₅₈H₇₅N₈O₁₇ [M+H]⁺: 1155.5245. Found: 1155.5240.

Fmoc-peg4-Gly-Cit-PABC-PNP (S5c). 7.3 mg, 47% for the 2 steps. White powder. HRMS (ESI) Calcd. For C₄₇H₅₆N₇O₁₅ [M+H]⁺: 958.3829. Found: 958.3811.

Fmoc-peg3-Glu-Gly-Cit-PABC-MMAE (S6a). Compound S5a (25.1 mg, 22.8 μmol) was dissolved in 20% TFA/DCM (200 μL and 800 μL). After being stirred at room temperature for 40 min, the solution was concentrated in vacuo and the crude peptide was precipitated with cold diethyl ether (20 mL) followed by centrifugation at 2,000×g for 3 min (3 times). The resulting crude peptide was dried in vacuo and then used immediately in the next step without purification. The crude peptide was dissolved in DMF (500 μL) and to the solution were added MMAE (18 mg, 25.1 μmol), DIPEA (7.9 μL, 45.6 μmol), and 1-hydroxy-7-azabenzotriazole (HOAt, 6.2 mg, 45.6 μmol). The mixture was stirred at 37° C. for 4 h and purified by preparative RP-HPLC under acidic conditions to afford S6a (20.9 mg, 57% for the 2 steps). White powder. HRMS (ESI) Calcd. For C₈₃H₁₂₁N₁₂O₂₁ [M+H]⁺: 1621.8764. Found: 1621.8722. Peptides S6b and S6c were synthesized from S5b and S5c in a similar manner.

Fmoc-peg3-Glu-Val-(N-Me)Cit-PABC-MMAE (S6b). 3.6 mg, 30% for the 2 steps. White powder. HRMS (ESI) Calcd. For C₈₇H₁₂₉N₁₂O₂₁ [M+H]⁺: 1667.9390. Found: 1667.9362.

Fmoc-peg4-Gly-Cit-PABC-MMAE (S6c). 6.5 mg, 56%. White powder. HRMS (ESI) Calcd. For C₈₀H₁₁₈N₁₁O₁₉ [M+H]⁺: 1536.8600. Found: 1536.8584.

BCN-peg3-Glu-Gly-Cit-PABC-MMAE (S7a). Compound S6a (10.5 mg, 6.5 μmol) was dissolved in 50% diethylamine/DMF solution (800 μL) at room temperature. After 1 h, the solution was concentrated in vacuo and used in the next step without further purification. BCN-NHS (2.3 mg, 7.8 μmol, Berry&Associates) and DIPEA (2.3 μL, 13 μmol) were added to a solution of this crude mixture in DMF (400 μL) and the mixture was stirred at room temperature for 3 h. The crude products were purified by preparative RP-HPLC under basic conditions to afford analytically pure peptide S7a (7.5 mg, 73% for the 2 steps). Purity was confirmed by LC-MS. White powder. HRMS (ESI) Calcd. For C₇₉H₁₂₃N₁₂O₂₁ [M+H]⁺: 1575.8920. Found: 1575.8909. Modules S7b and S7c were synthesized from S6b and S6c in a similar manner.

BCN-peg3-Glu-Val-(N-Me)Cit-PABC-MMAE (S7b). 1.2 mg, 35% for the 2 steps. White powder. HRMS (ESI) Calcd. For C₈₃H₁₃₁N₁₂O₂₁ [M+H]⁺: 1631.9546. Found: 1631.9529.

BCN-peg4-Gly-Cit-PABC-MMAE (S7c). 4.1 mg, 65% for the 2 steps. White powder. HRMS (ESI) Calcd. For C₇₆H₁₂₀N₁₁O₁₉ [M+H]⁺: 1490.8757. Found: 1490.8737.

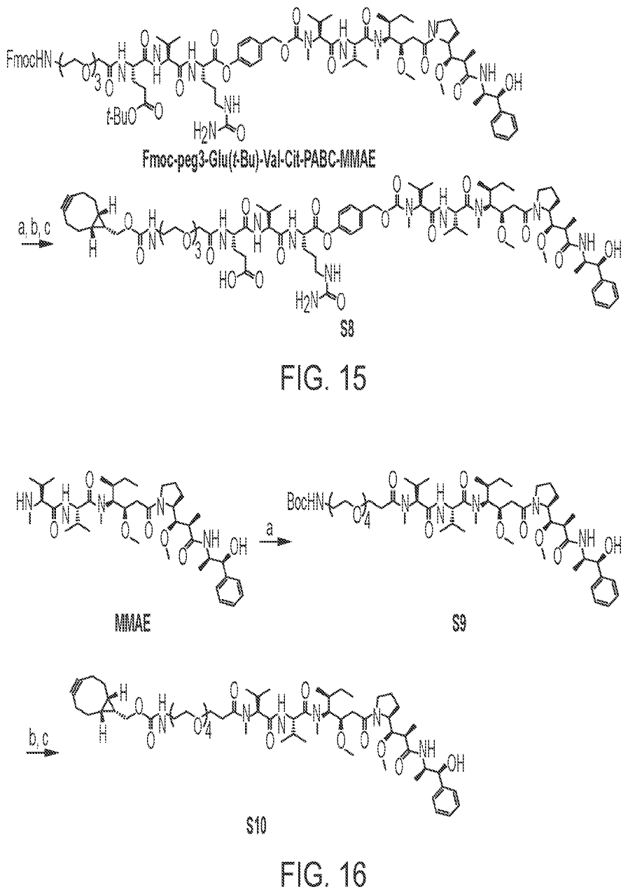

BCN-peg3-Glu-Val-Cit-PABC-MMAE (S8). (FIG. 15) Fmoc-peg₃-Glu(t-Bu)-Val-Cit-PABC-MMAE. (7.3 mg, 4.2 μmol, prepared as described previously)³ was added in 20% TFA/DCM solution at room temperature. After 1 h, the solution was concentrated in vacuo and the crude compounds were precipitated with cold diethyl ether (20 mL) followed by centrifugation at 2,000 g for 3 min (3 times). The crude products were dissolved in 50% diethylamine/DMF solution. After being stirred at room temperature for 1 h, the solution was concentrated in vacuo and used in the next step without further purification. BCN-NHS (2.5 mg, 8.4 μmol) and DIPEA (2.2 μL, 12.6 μmol) was added to a solution of this crude mixture in DMF and the mixture was stirred at room temperature overnight. The crude products were purified by preparative RP-HPLC under basic conditions to afford S8 (4.2 mg, 61% for the 3 steps). Purity was confirmed by LC-MS. White powder. HRMS (ESI) Calcd. For C82H₁₂₈N₁₂O₂₁Na₂ [M+2Na]²⁺: 831.4551. Found: 831.4577. (FIG. 15) Boc-peg4-MMAE (S9). Boc-peg4 acid (5.3 mg, 14.6 μmol) in DMF (150 μL) was mixed with HATU (11 mg, 29.1 μmol) and DIPEA (7.6 μL, 43.7 μmol) and stirred for 5 min to activate it. Then the mixture was added to a solution of MMAE (7 mg, 9.7 μmol) in DMF (450 μL) and stirred at room temperature for 1 h. The crude products were purified by preparative RP-HPLC under acidic conditions to afford analytically pure peptide S9 (13 mg, quant.). Purity was confirmed by LC-MS. White powder. HRMS (ESI) Calcd. For C₅₅H₉₇N₆O₁₄ [M+H]⁺: 1065.7057. Found: 1065.7045.

BCN-peg4-MMAE (S10) (FIG. 16). Compound S9 (13 mg, 12.2 μmol) was dissolved in 50% TFA/DCM solution (1 mL). After being stirred at room temperature for 30 min, the solution was concentrated in vacuo and the crude compounds were precipitated with cold diethyl ether (20 mL) followed by centrifugation at 2,000 g for 3 min (3 times). BCN-NHS (0.3 mg, 1.0 μmol) and DIPEA (0.25 μL, 1.4 μmol) was added to a solution of this crude mixture in DMF (500 μL) and the mixture was stirred at room temperature for 30 min. The crude products were purified by preparative RP-HPLC under FA conditions to afford S10 (1.8 mg, 13% for the 2 steps). Purity was confirmed by LC-MS. White powder. HRMS (ESI) Calcd. For C₆₁H₁₀₁N₆O₁₄ [M+H]⁺: 1141.7370. Found: 1141.7361.

Fmoc-peg3-Glu-Gly-Cit-PABC-MMAF (S11). Compound S5a (7.4 mg, 6.7 μmol) was dissolved in 20% TFA/DCM (200 μL and 800 μL). After being stirred at room temperature for 50 min, the solution was concentrated in vacuo and the crude peptide was precipitated with cold diethyl ether (20 mL) followed by centrifugation at 2,000×g for 3 min (3 times). The resulting crude peptide was dried in vacuo and then used immediately in the next step without purification. The crude peptide was dissolved in DMF (350 μL) and to the solution were added MMAF·TFA salt (7.4 mg, 8.7 μmol), DIPEA (2.3 μL, 13.4 μmol), and HOAt (1.8 mg, 13.4 μmol). The mixture was stirred at 37° C. overnight and purified by preparative RP-HPLC under acidic conditions to afford S11 (9.4 mg, 85% for the 2 steps). White powder. HRMS (ESI) Calcd. For C₈₃H₉N₁₂O₂₂ [M+H]⁺: 1635.8556, Found: 1635.8541.

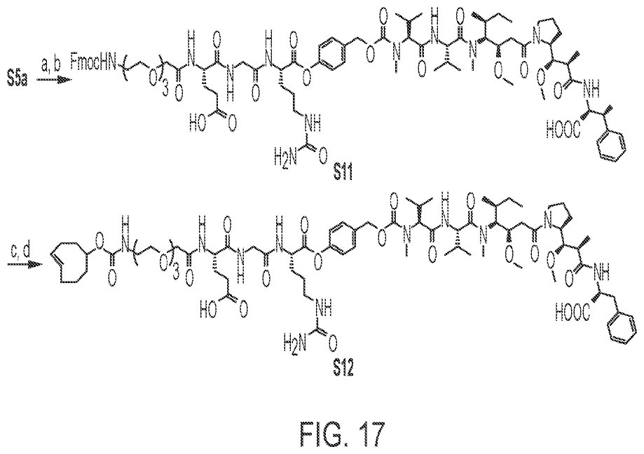

BCN-peg3-Glu-Gly-Cit-PABC-MMAF (S12) (FIG. 17). Compound S11 (4.7 mg, 2.9 μmol) was dissolved in 50% diethylamine/DMF solution (600 μL) at room temperature. After 30 min, the solution was concentrated in vacuo and used in the next step without further purification. BCN-NHS (1.1 mg, 3.8 μmol) and DIPEA (1 μL, 5.8 μmol) were added to a solution of this crude mixture in DMF (300 μL) and the mixture was stirred at room temperature for 3.5 h. The crude products were purified by preparative RP-HPLC under basic conditions to afford analytically pure peptide S12 (1.5 mg, 33% for the 2 steps). Purity was confirmed by LC-MS. White powder. HRMS (ESI) Calcd. For C₇₇H₁₂₁N₁₂O₂₂ [M+H]⁺: 1565.8713, Found: 1565.8671.

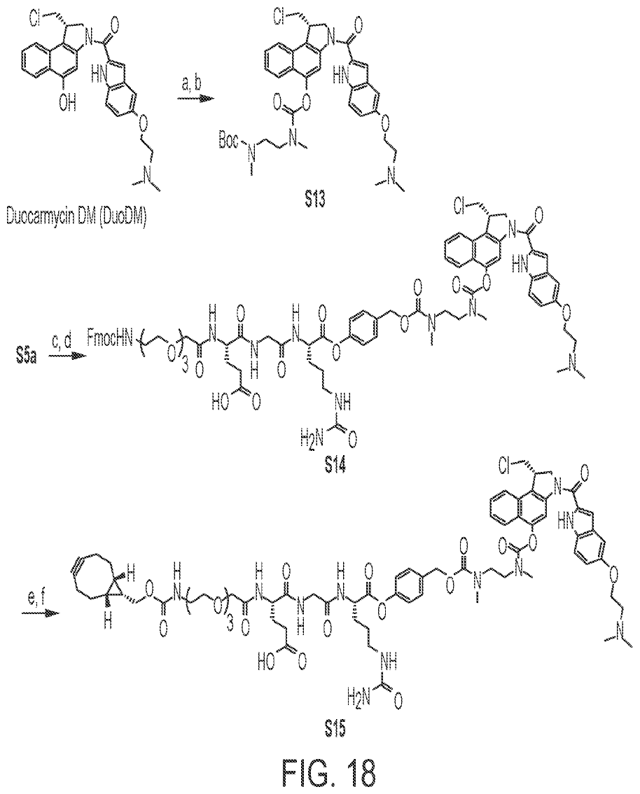

Boc-protected duocarmycin DM (S13) (FIG. 18). To a solution of duocarmycin DM (DuoDM, 7.0 mg, 12.1 μmol) in dry ACN (800 μL) were added DIPEA (6.3 μL, 36.3 μmol) and 4-nitrophenyl chloroformate (4.9 mg, 24.2 μmol). After being stirred at room temperature for 30 min, t-butyl methyl(2-(methylamino)ethyl)carbamate (12 μL, 60.5 μmol, AK Scientific) was added to the mixture and the mixture was stirred at room temperature for 15 min. The reaction was quenched with 2-N HCl/ACN at 0° C. and the crude products were purified by preparative RP-HPLC to afford analytically pure compound S13 (8.0 mg, 84% for the 2 steps). Purity was confirmed by LC-MS. White powder. HRMS (ESI) Calcd. For C₃₆H₄₅N₅O₆Cl [M+H]⁺: 678.3053, Found: 678.3029.

Fmoc-peg3-Glu-Gly-Cit-PABC-DuoDM (S14). Compound S5a (10.4 mg, 9.5 μmol) was dissolved in 20% TFA/DCM (200 μL and 800 μL). After being stirred at room temperature for 50 min, the solution was concentrated in vacuo and the crude peptide was precipitated with cold diethyl ether (20 mL) followed by centrifugation at 2,000×g for 3 min (3 times). In parallel, compound S13 (8.0 mg, 10.1 μmol) was dissolved in 50% TFA/DCM (500 μL and 800 μL) at 0° C. and the mixture was stirred for 30 min. The solution was concentrated in vacuo and the crude compound was precipitated with cold diethyl ether (20 mL) followed by centrifugation at 2,000×g for 3 min (3 times). The crude peptide and Boc-deprotected compound were dissolved in DMF (600 μL) and DIPEA (8.2 μL, 47.5 μmol) was added to the mixture at 0° C. The mixture was stirred at room temperature for 2 h and the reaction was quenched with 2-N HCl/ACN at 0° C. The crude products were purified by preparative RP-HPLC under acidic conditions to afford S14 (8.7 mg, 58% for the 2 steps). White powder. HRMS (ESI) Calcd. For C₇₅H₉₀N₁₂O₁₈Cl [M+H]⁺: 1481.6179, Found: 1481.6165.

BCN-peg3-Glu-Gly-Cit-PABC-DuoDM (S15). Compound S14 (8.7 mg, 5.5 μmol) was dissolved in 50% diethylamine/DMF solution (600 μL) at room temperature. After 30 min, the solution was concentrated in vacuo and used in the next step without further purification. BCN-NHS (2.1 mg, 7.2 μmol) and DIPEA (1.9 μL, 11 μmol) were added to a solution of this crude mixture in DMF (300 μL) and the mixture was stirred at room temperature for 1 h. The crude products were purified by preparative RP-HPLC under basic conditions to afford analytically pure peptide S15 (3.5 mg, 44% for the 2 steps). Purity was confirmed by LC-MS. White powder. HRMS (ESI) Calcd. For C₇₁H₉₂N₁₂O₁₈Cl [M+H]⁺: 1435.6336, Found: 1435.6329.

seco-CBI-β-glucuronide (S16). A suspension of Boc-seco-CBI (10 mg, 30 μmol), methyl-(2,3,4-tri-O-acetyl-α-D-glucopyranosyl trichloroacetimidate (21.5 mg, 45 μmol, AmBeed), and molecular sieves 4 Å (50 mg) in DCM (1 mL) was stirred at room temperature for 30 min. The mixture was cooled to −20° C. and BF₃·Et₂O (3.3 μL, 15 μmol, ca. 48% BF₃) in DCM (100 μL) was added dropwise. After being stirred at −20° C. for 2 h, additional BF₃·Et₂O (20 μL, 90 μmol, ca. 48% BF₃) in DCM (100 μL) was added dropwise, then the mixture was warmed to room temperature and stirred at room temperature for 2 h. The reaction was quenched by filtration over a celite pad. The crude products were purified by preparative RP-HPLC under FA conditions to afford analytically pure compound S16 (13.5 mg, 82%). Purity was confirmed by LC-MS. Off-white powder. HRMS (ESI) Calcd. For C₂₆H₂₉NO₁₀Cl [M+H]⁺: 550.1475, Found: 550.1464.

Duocarmycin DM-β-glucuronide (S17). Compound S16 (5.4 mg, 9.8 μmol) in DMF (400 μL) was mixed with 5-(2-dimethylaminoethoxy)indole-2-carboxylic acid (7.3 mg, 29.4 μmol, prepared as described previously)⁴ and N-(3-dimethylaminopropyl)-N′-ethylcarbodiimide hydrochloride (EDC·HCl, 11.2 mg, 58.5 μmol) at room temperature, them the reaction mixture was stirred at room temperature for 1.5 h. The crude products were purified by preparative RP-HPLC under FA conditions to afford analytically pure compound S17 (5.9 mg, 77%). Purity was confirmed by LC-MS. Off-white powder. HRMS (ESI) Calcd. For C₃₉H₄₃N₃O₁₂Cl [M+H]⁺: 780.2530, Found: 780.2494.

Fmoc-peg3-Glu(t-Bu)-Gly-Cit-PAB-Cl (S18). Fmoc-peg3-Glu(t-Bu)-Gly-Cit-PABOH (21.8 mg, 23 μmol, an intermediate of S5a) in dry DMF (300 μL) was cooled to ° C. and thionyl chloride (1.8 μL, 25 μmol) in dry DCM (100 μL) was added dropwise. After being stirred at 0° C. for 1 h, additional thionyl chloride (1.8 μL, 25 μmol) in DCM (100 μL) was added dropwise. After 1 h, additional thionyl chloride (1.8 μL, 25 μmol) in DCM (100 μL) was added dropwise, then the mixture was stirred at 0° C. for further 30 min. The crude products were purified by preparative RP-HPLC to afford peptide S18 (13.7 mg, ˜70% purity). NOTE: A single peak was collected by preparative RP-HPLC, however, the chloride compound S18 was hydrolyzed to the starting material during lyophilization. Thus, the purity of S18 was decreased to ˜70% after lyophilization. White powder. HRMS (ESI) Calcd. For C₄₇H₆₃N₇O₁₂Cl [M+H]⁺: 952.4218, Found: 952.4213.

Fmoc-peg3-Glu(t-Bu)-Gly-Cit-PAB-DuoDM-β-glucuronide (S19). Compound S18 (3.4 mg, 1.8 μmol, ˜70% purity) in dry DMF (250 μL) was mixed with compound S17 (0.9 mg, 1.2 μmol), sodium iodide (0.1 mg, 0.6 μmol), and DIPEA (0.4 μL, 2.4 μmol), then the mixture was stirred at room temperature for 6 h. Additional compound S17 (1.2 mg, 1.5 μmol) and DIPEA (0.4 μL, 2.4 μmol) was added to the mixture. After 2 h, the mixture was added additional compound S17 (0.6 mg, 0.75 μmol) and stirred at room temperature overnight. The reaction progress was monitored by LC-MS and the starting material S18 was still remained. The reaction mixture was warmed to 37° C. and added additional compound S17 (0.5 mg, 0.64 μmol). After 3 h, the crude products were purified by preparative RP-HPLC under acidic conditions to afford analytically pure compound S19 (2.9 mg, 95%). Purity was confirmed by LC-MS. White powder. HRMS (ESI) Calcd. For C₈₆H₁₀₄N₁₀O₂₄Cl [M]⁺: 1695.6908, Found: 1695.6901.

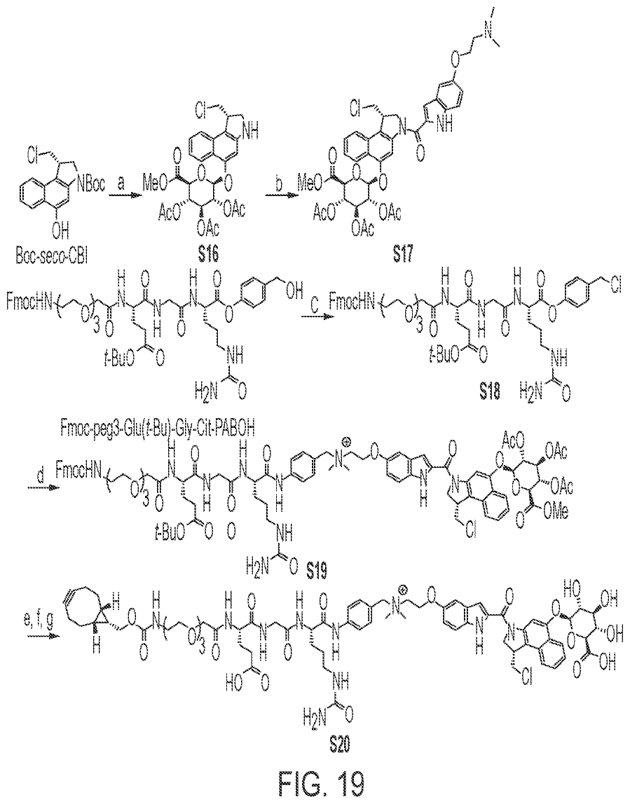

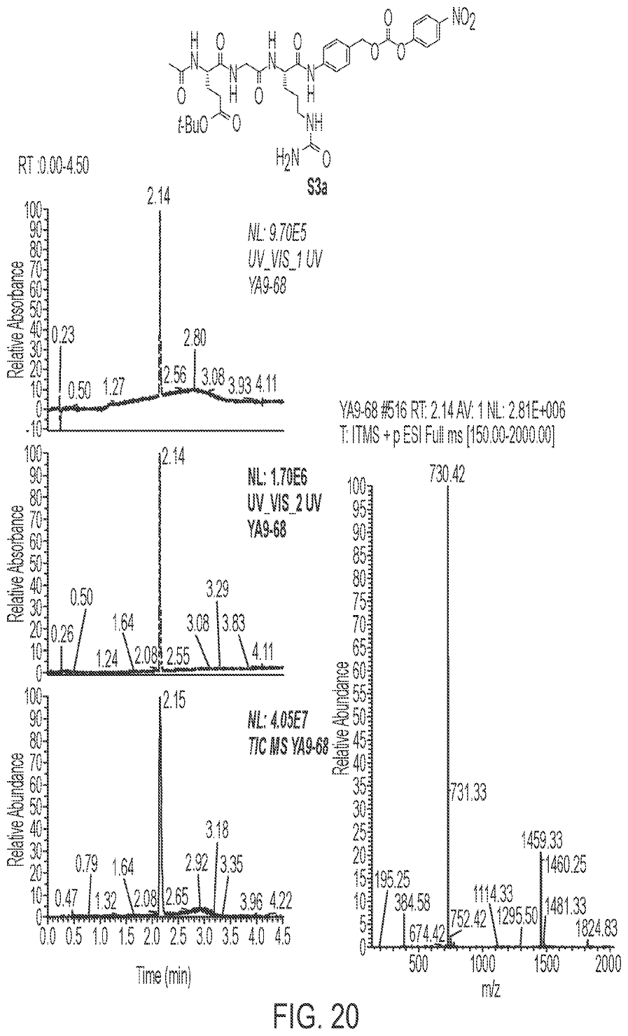

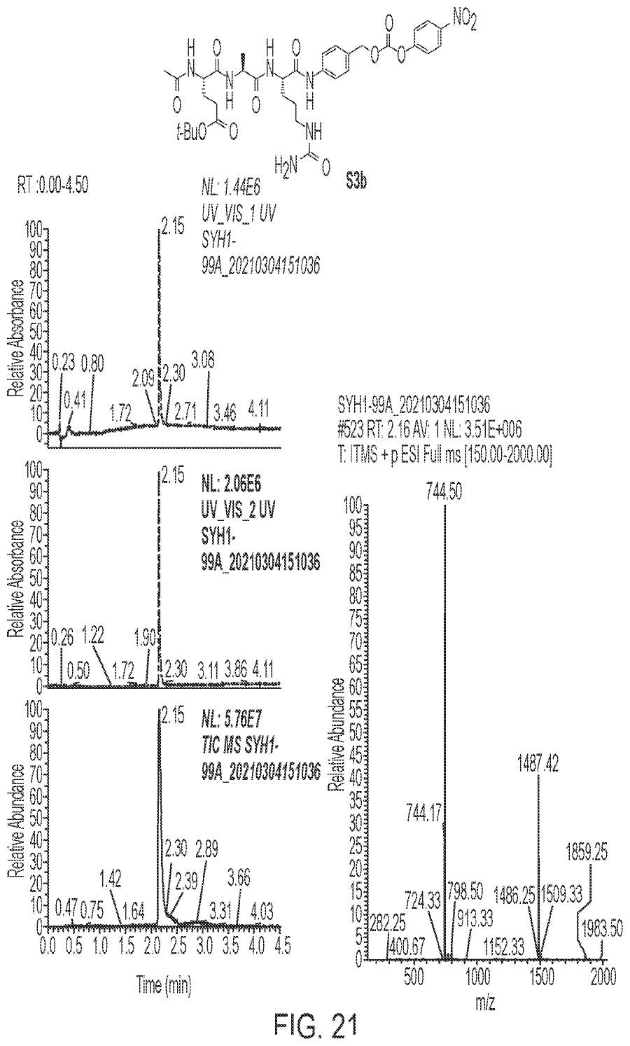

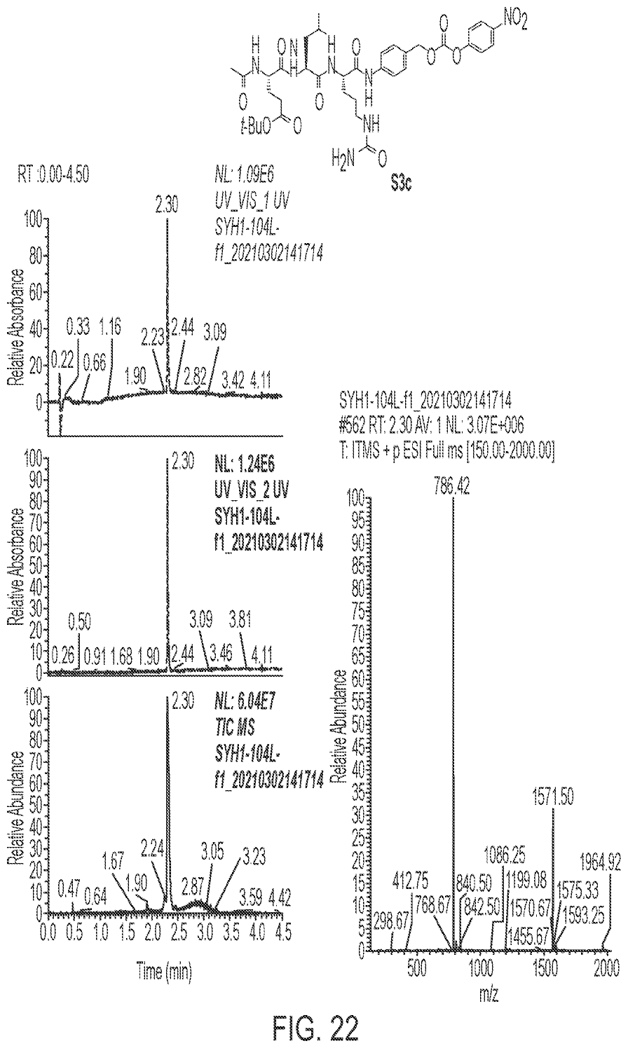

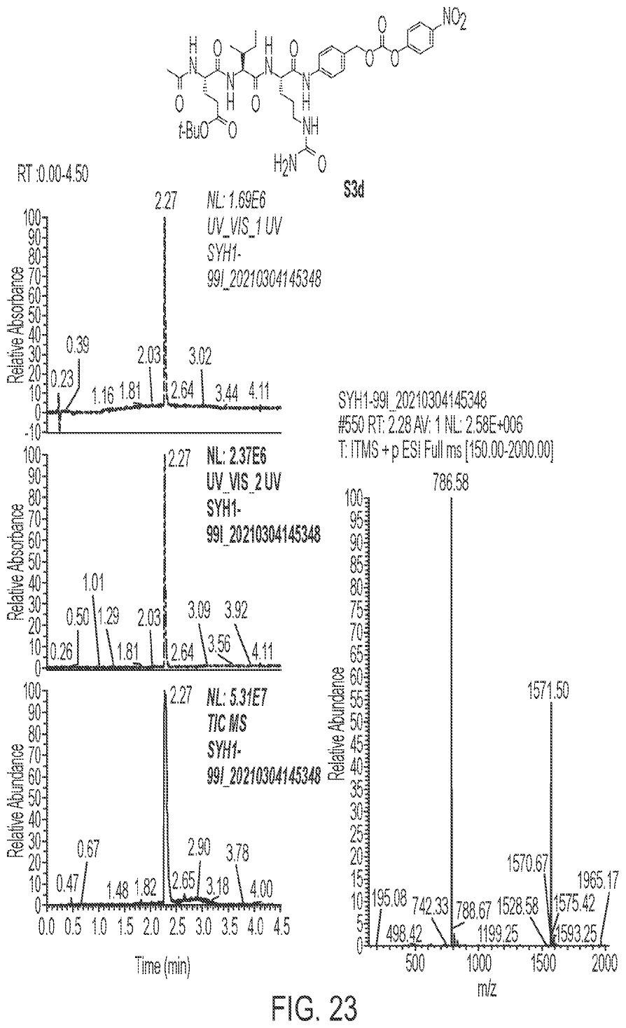

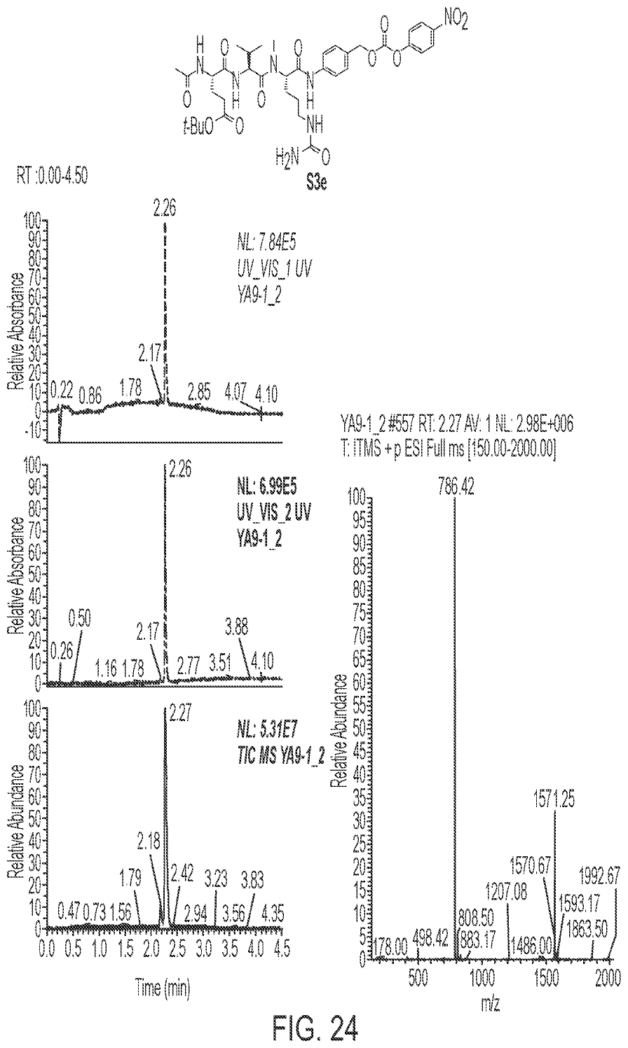

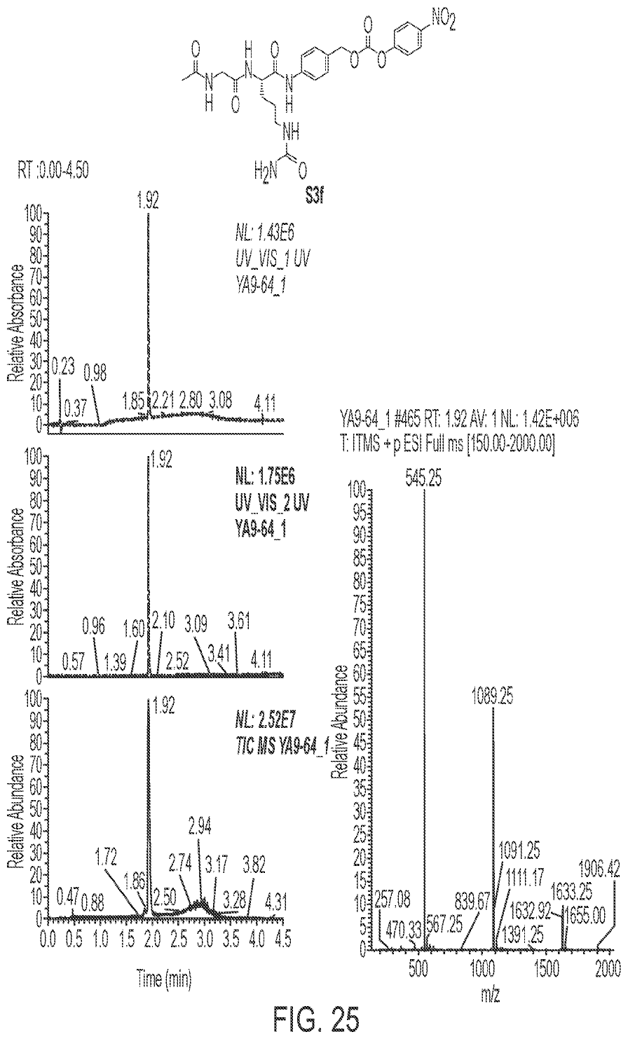

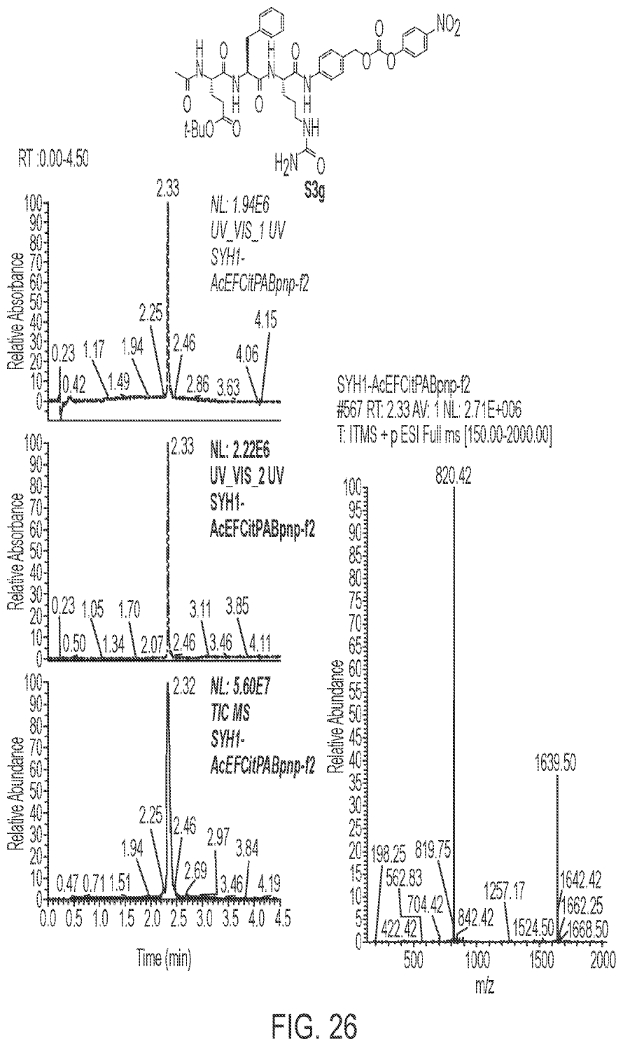

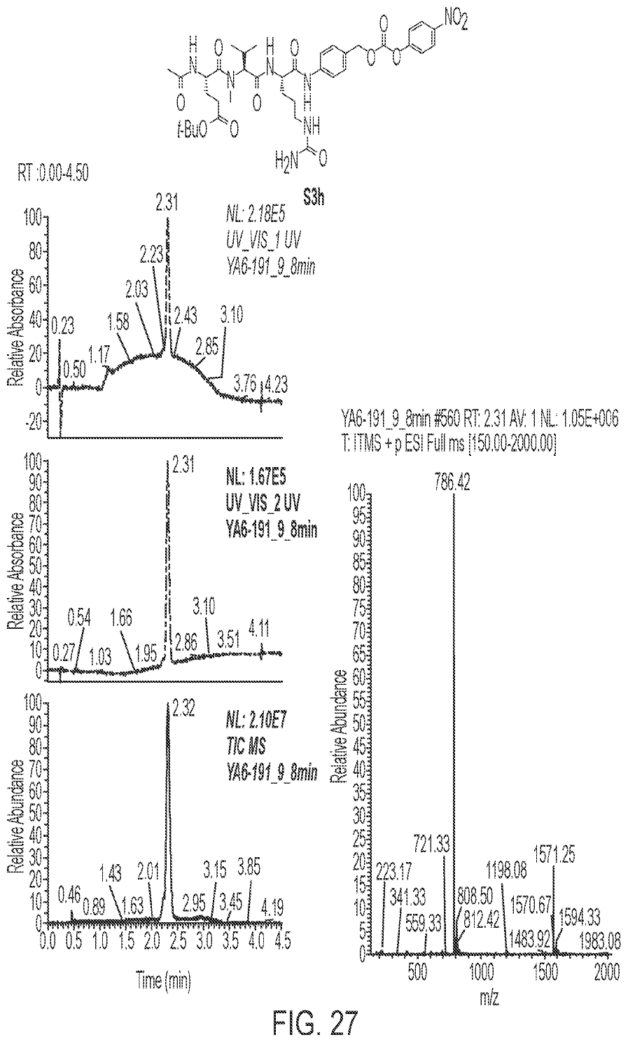

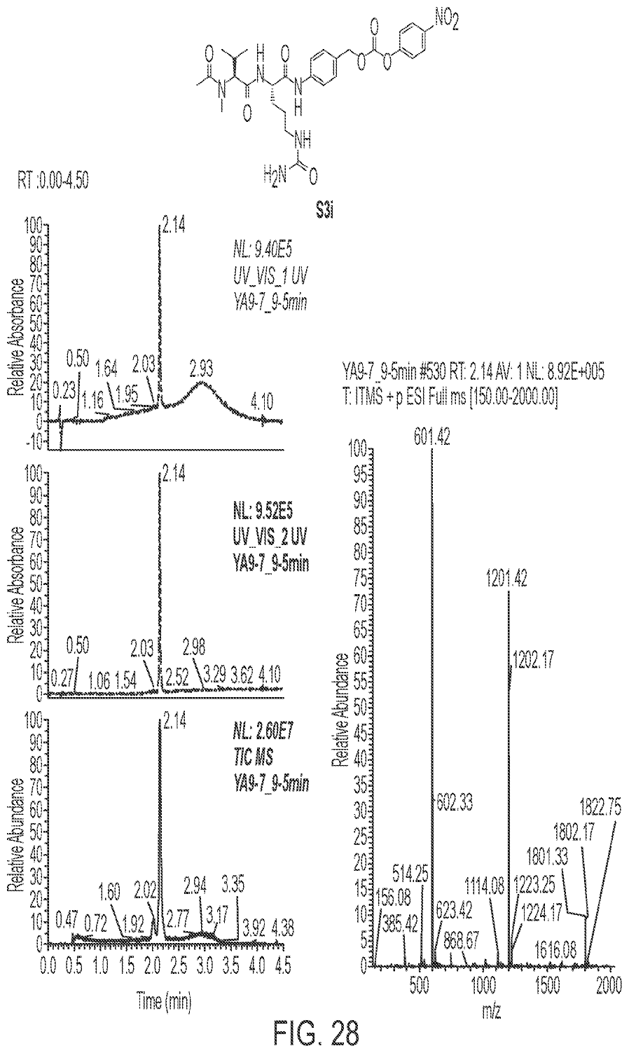

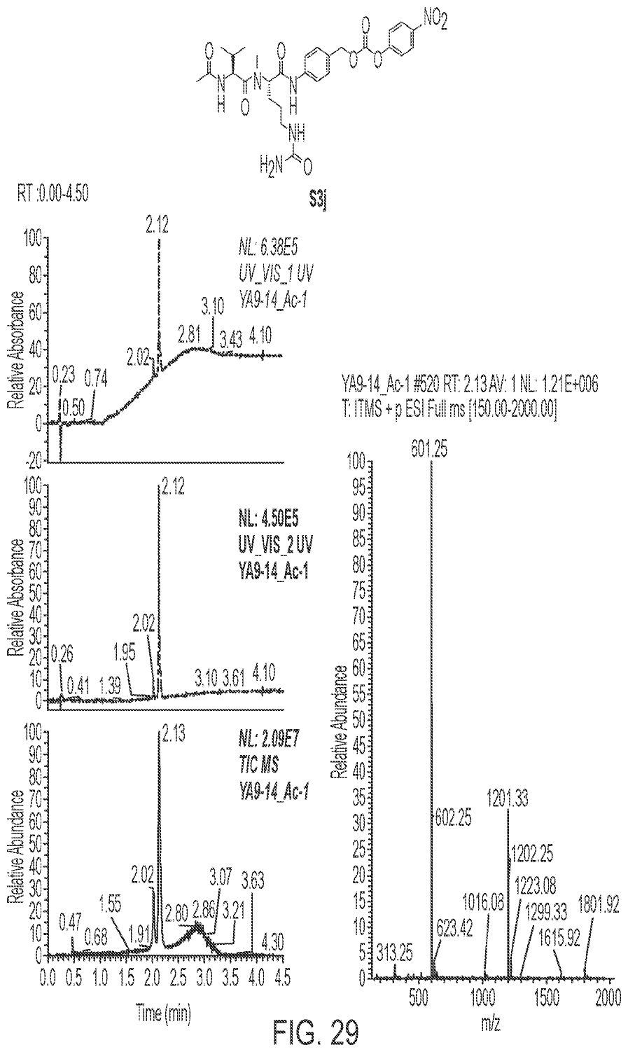

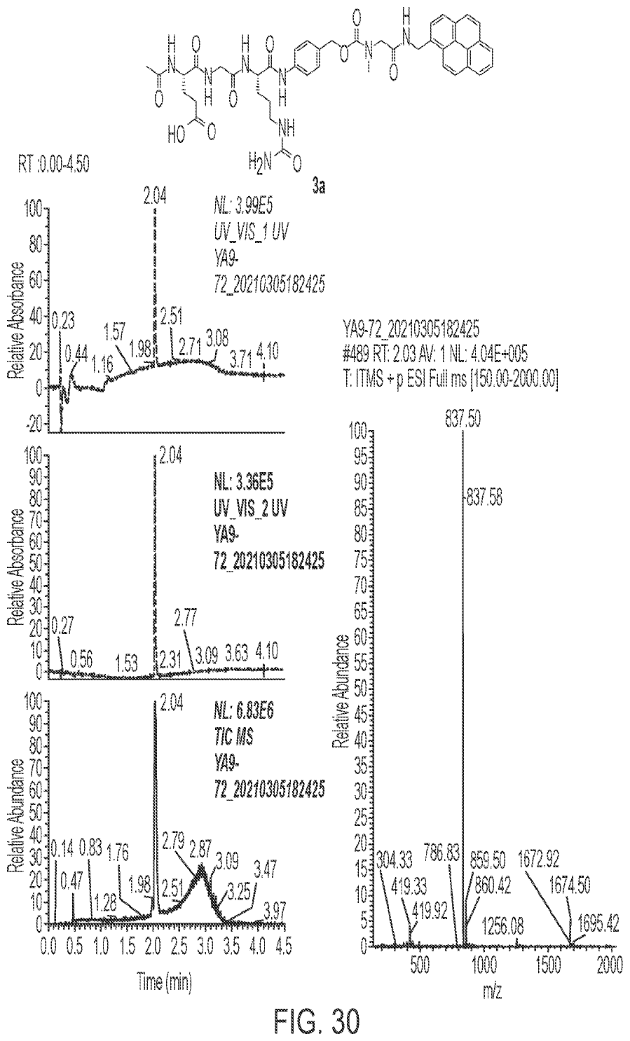

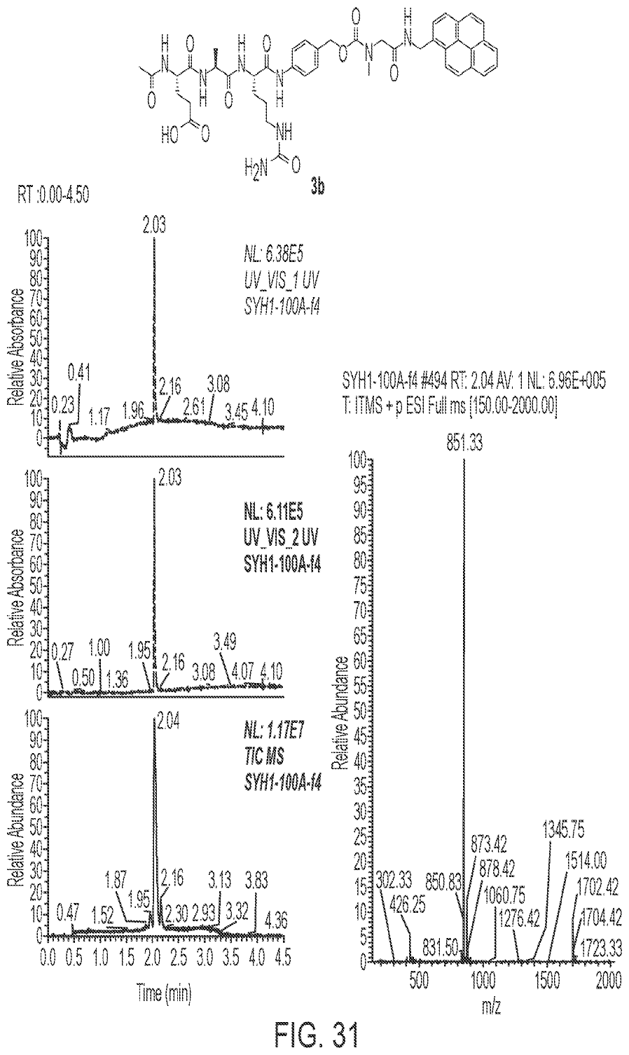

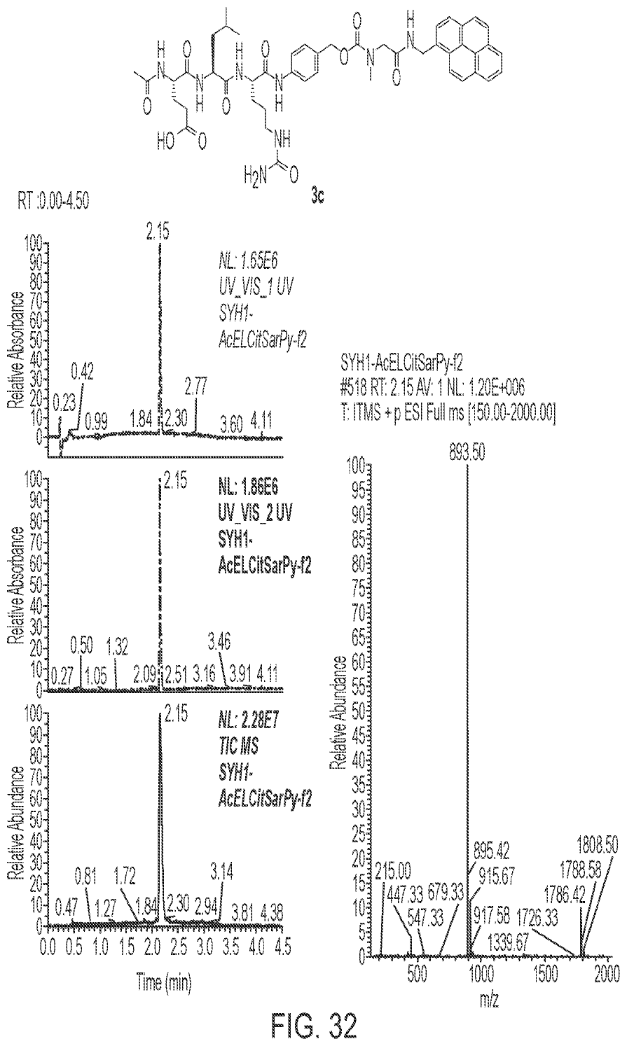

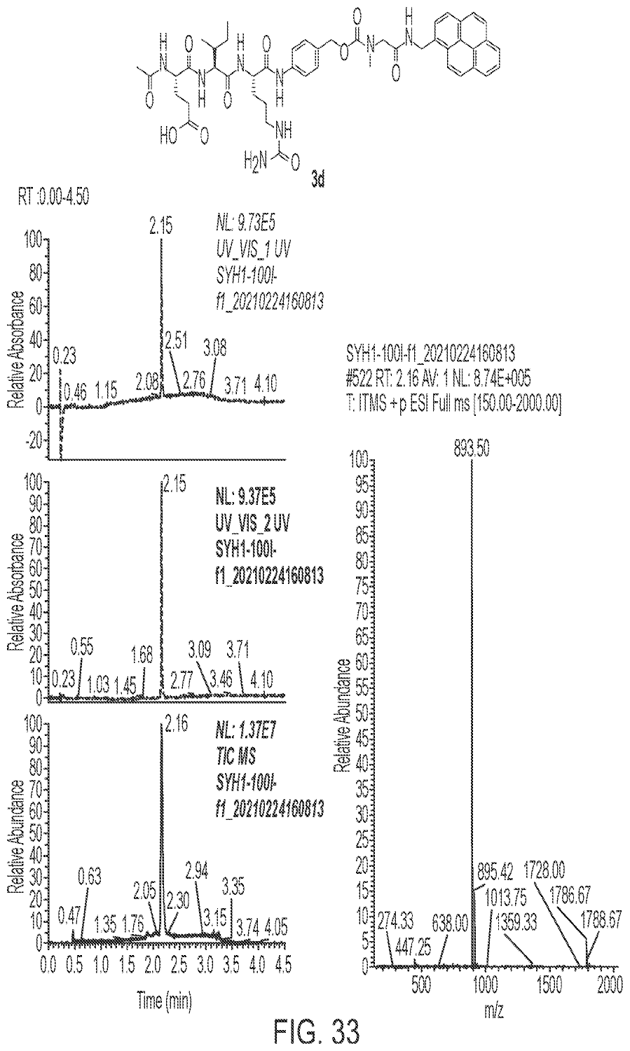

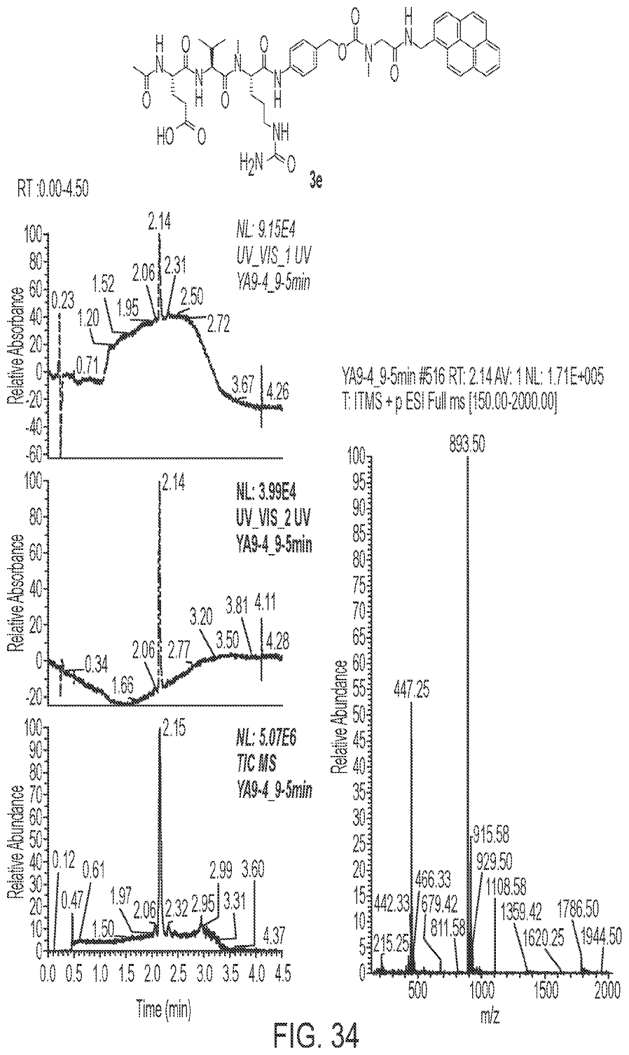

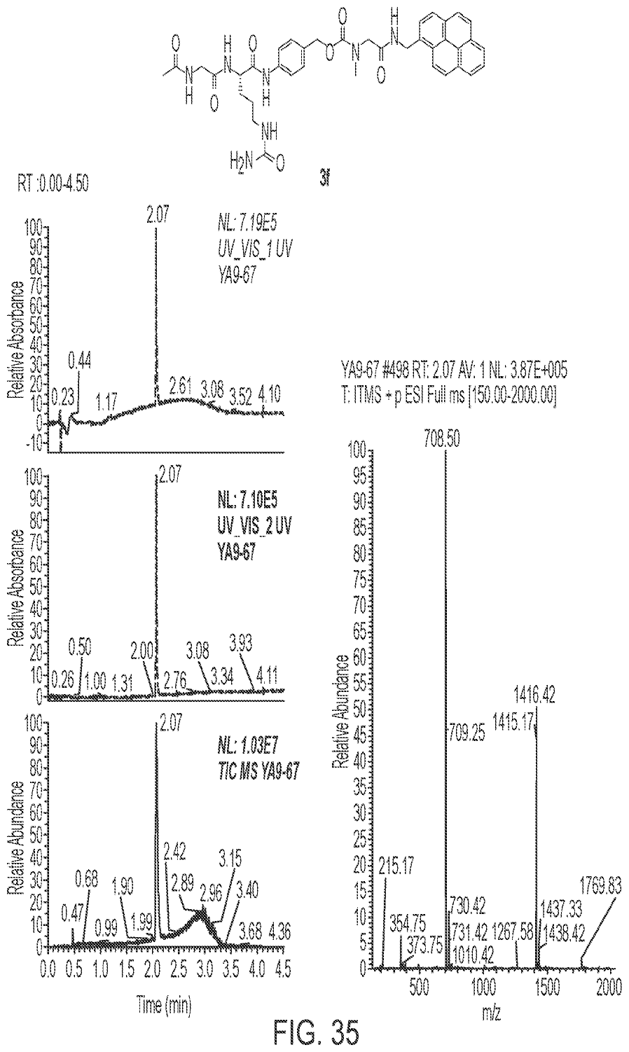

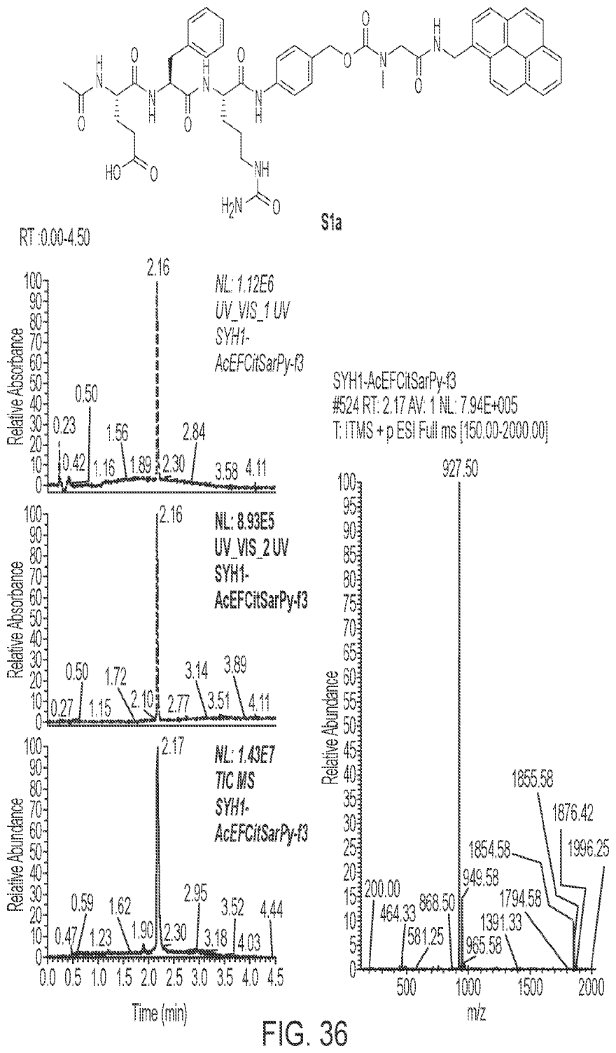

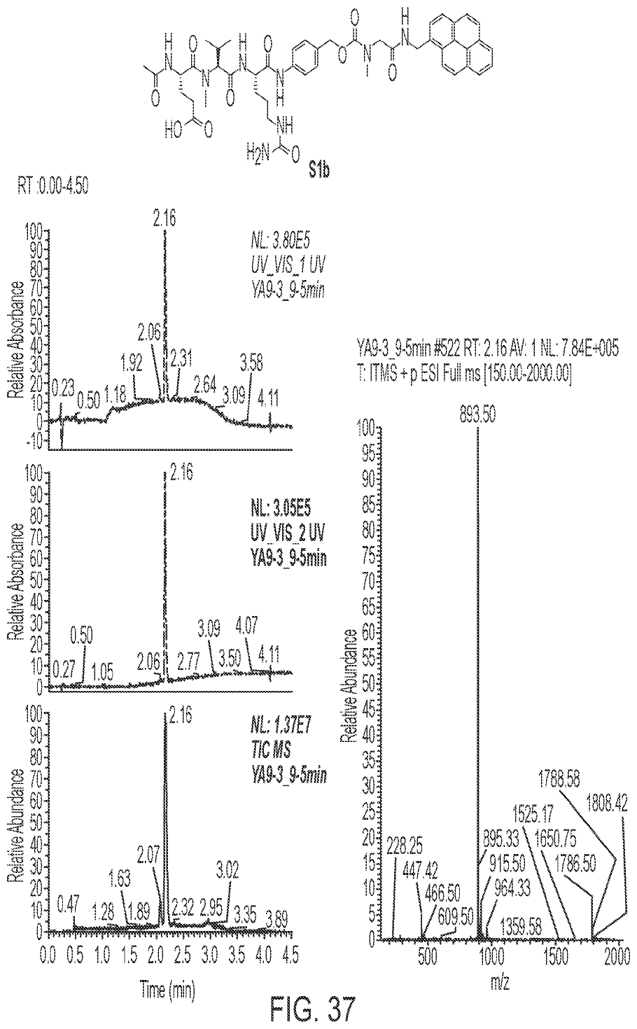

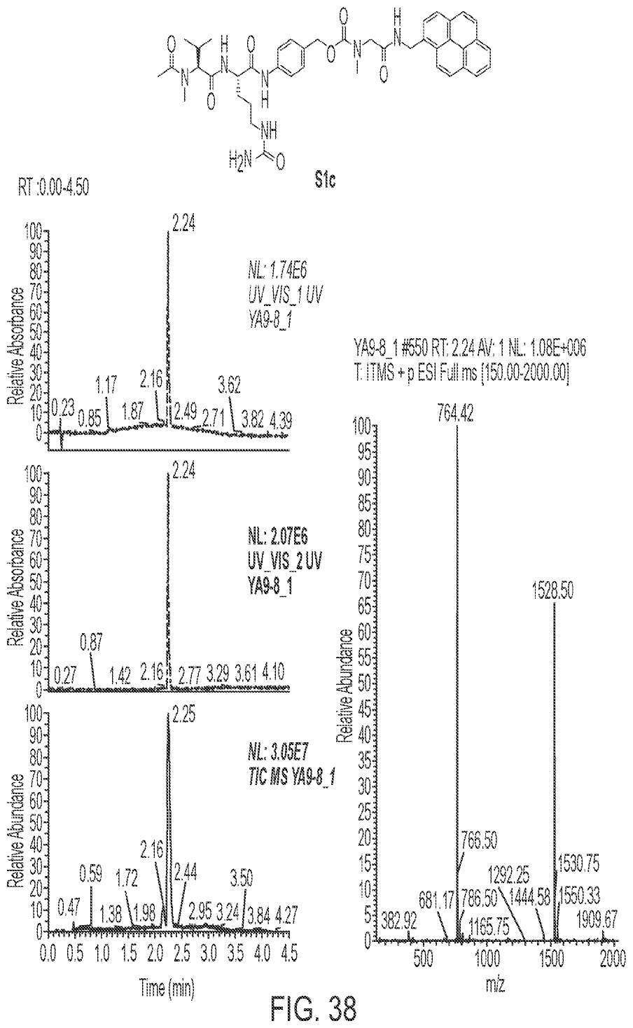

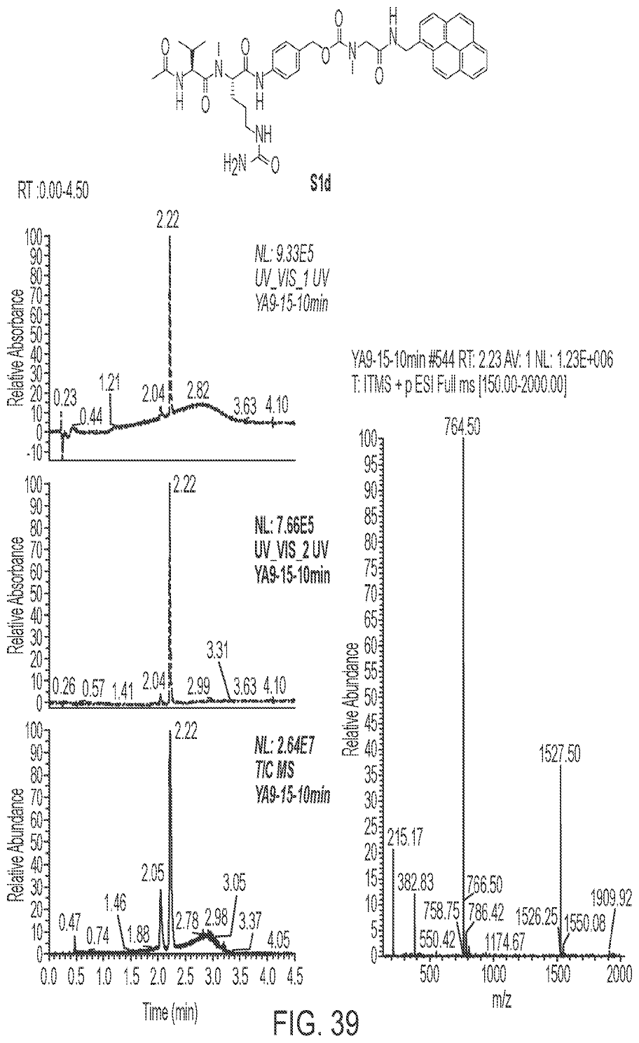

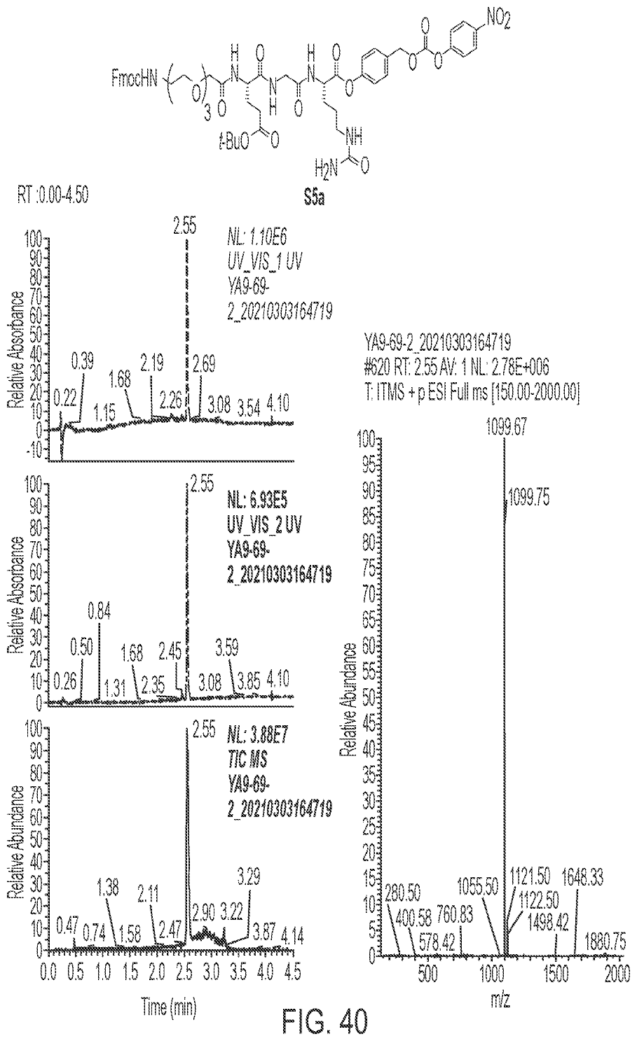

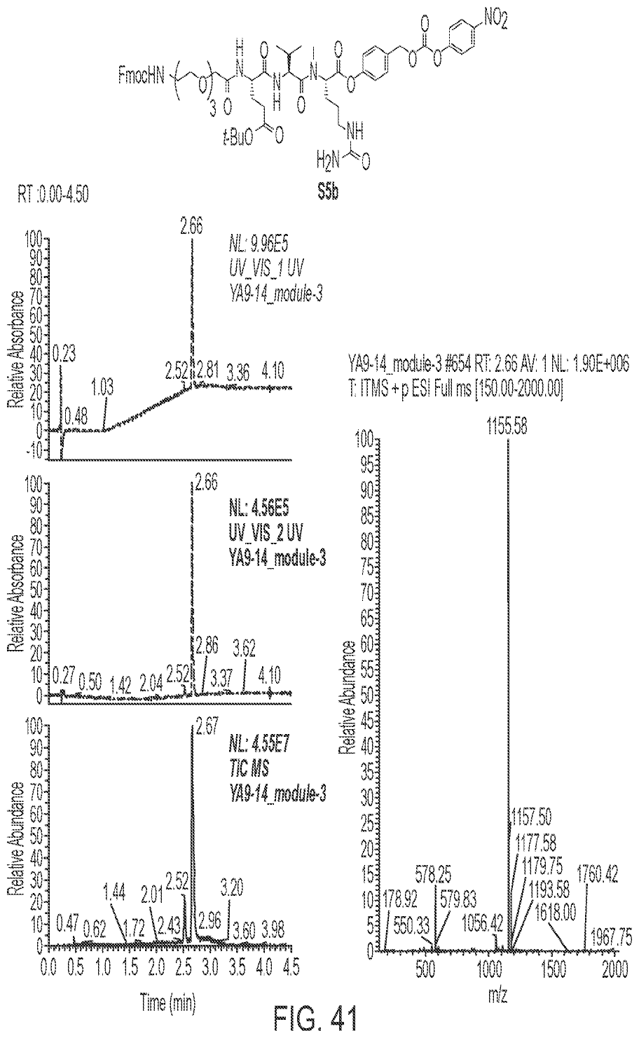

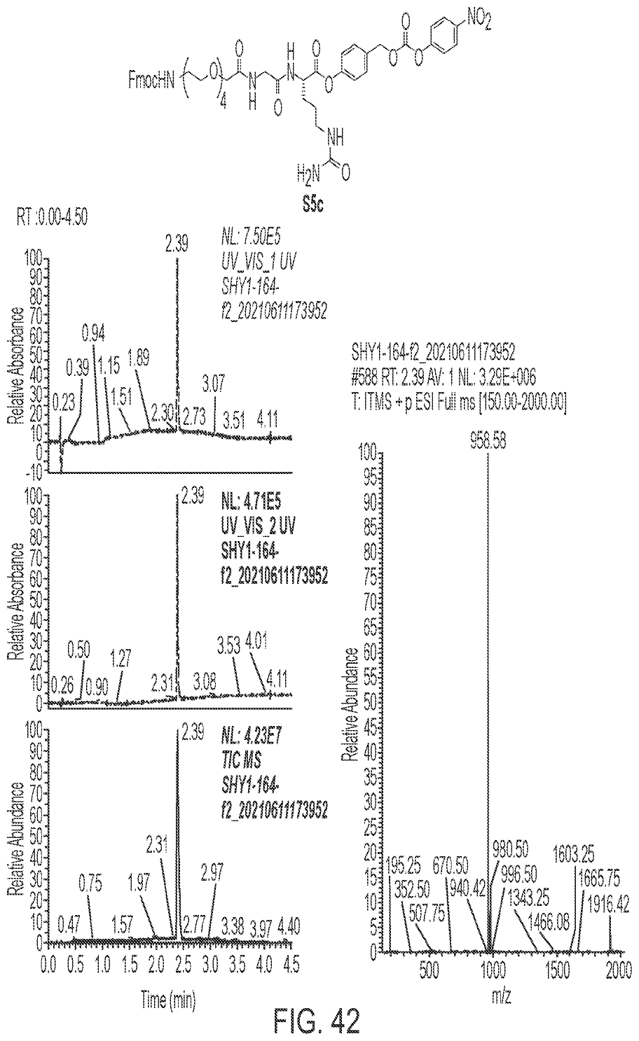

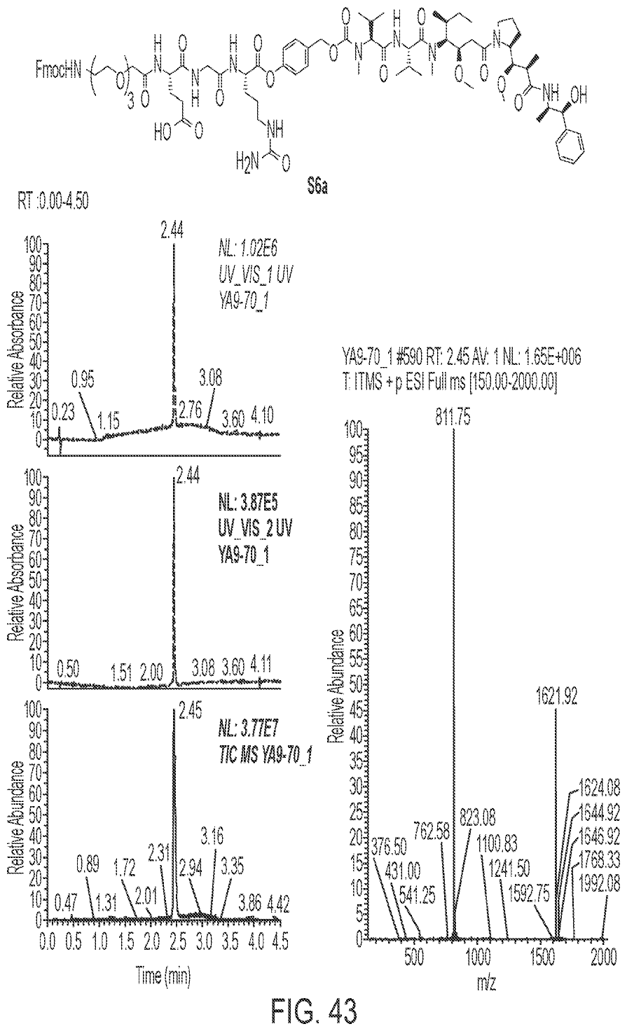

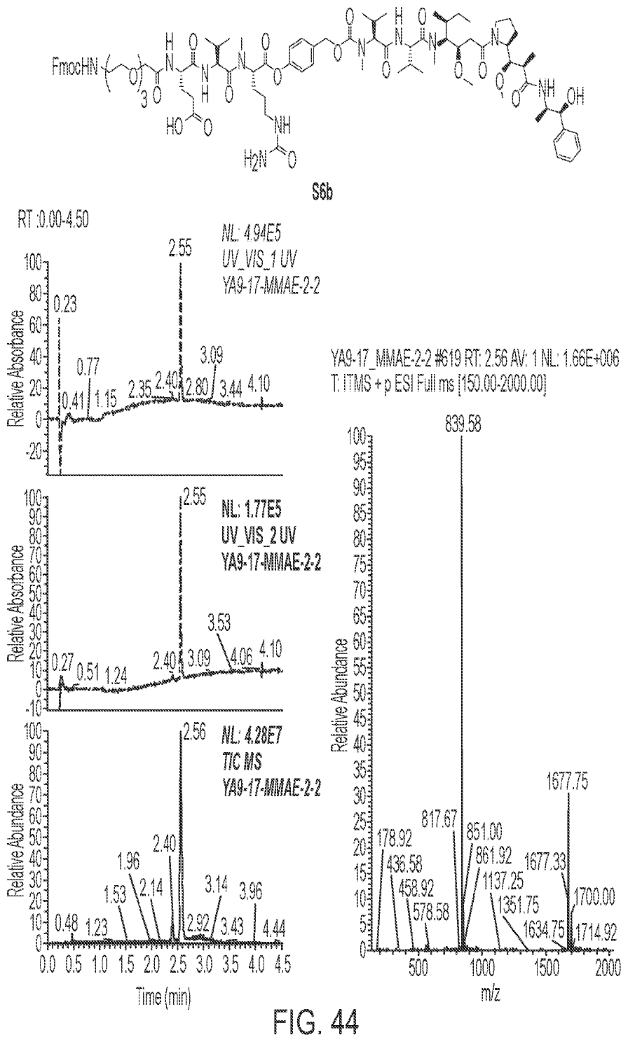

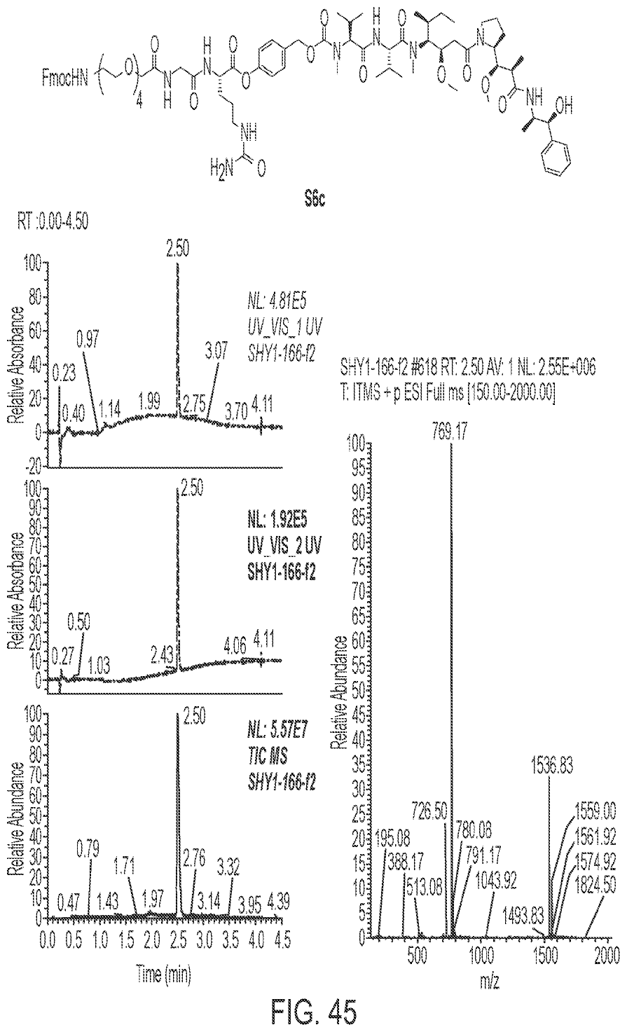

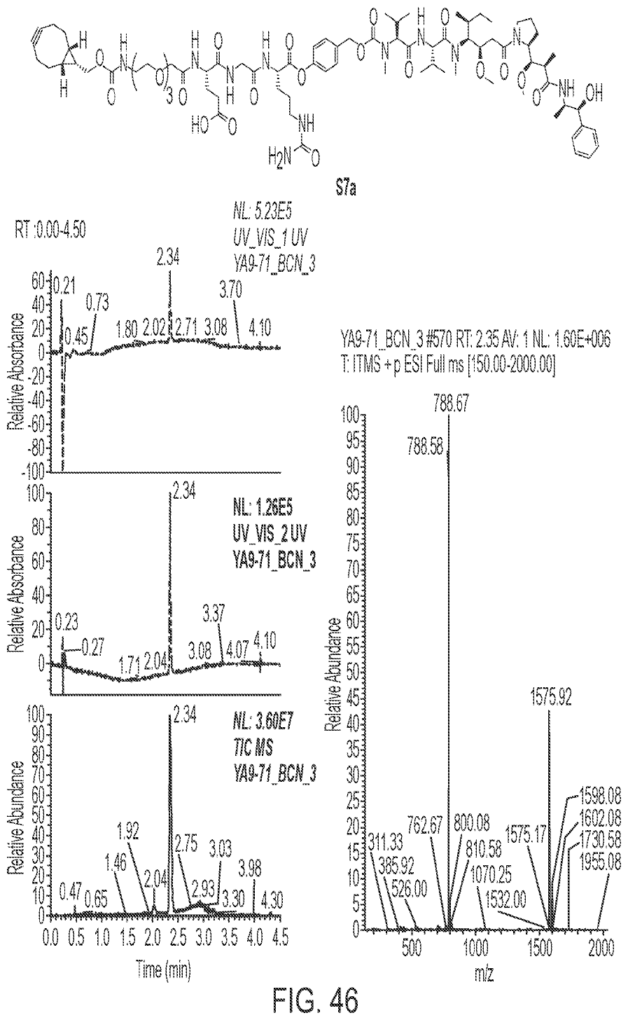

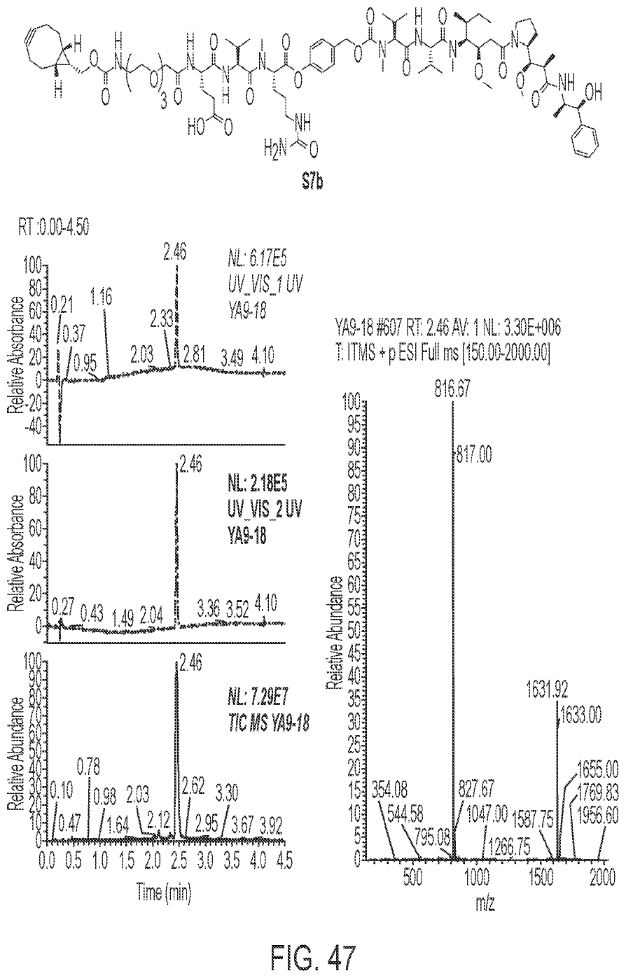

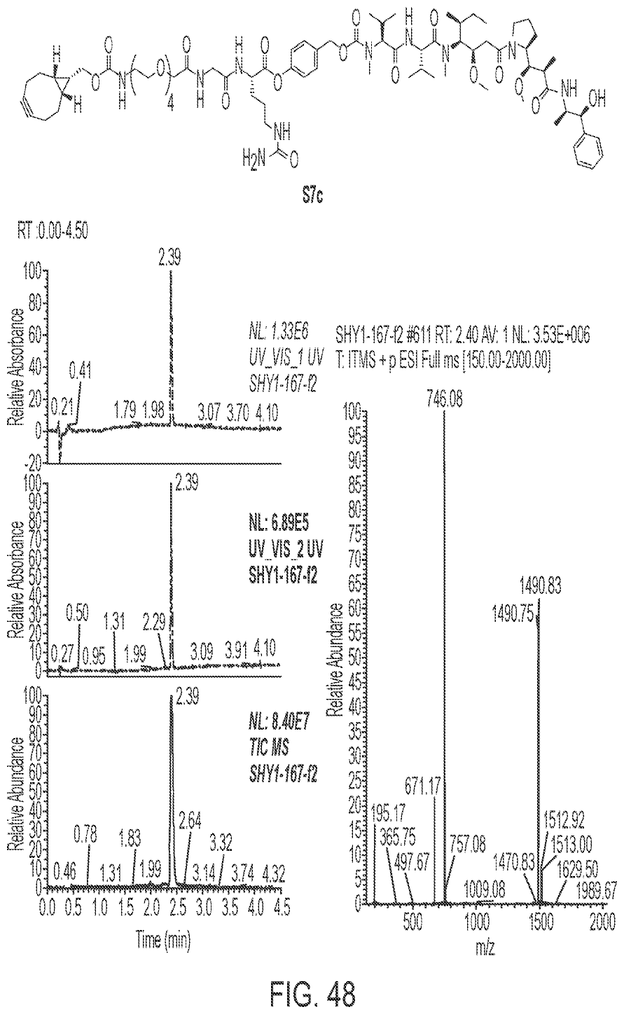

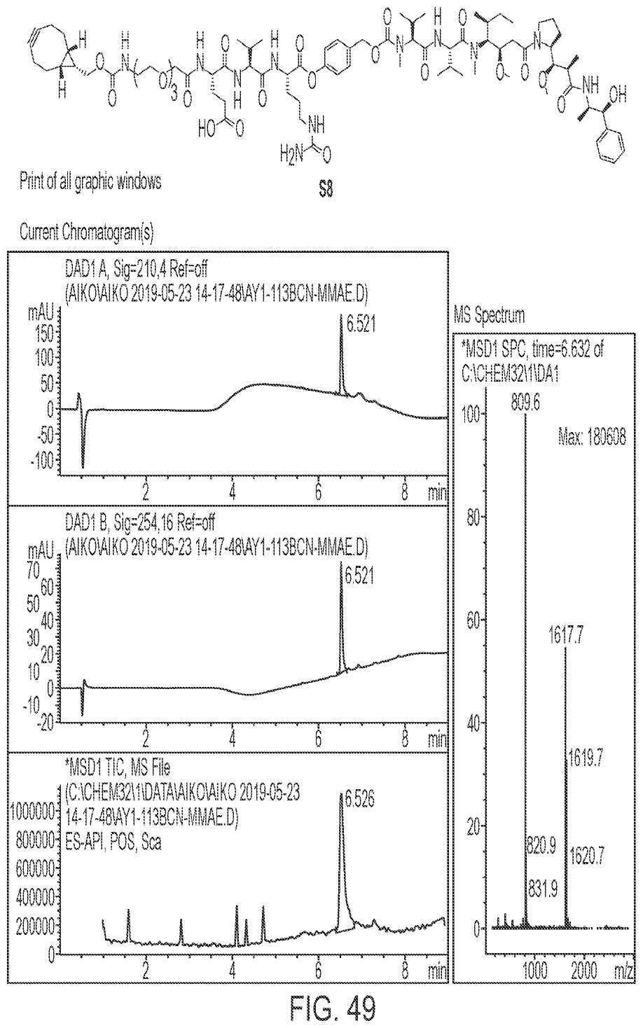

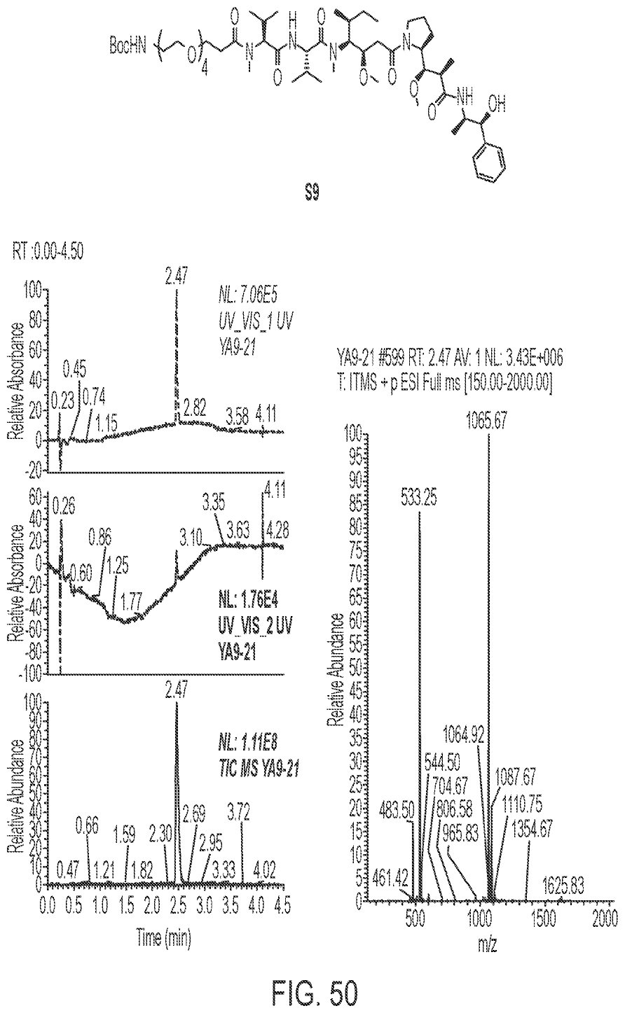

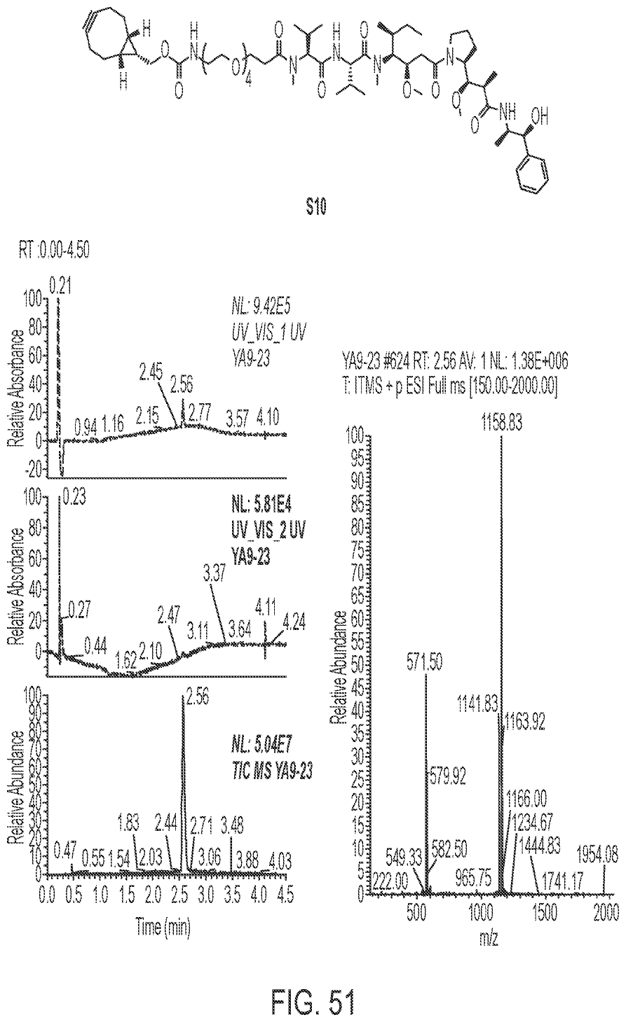

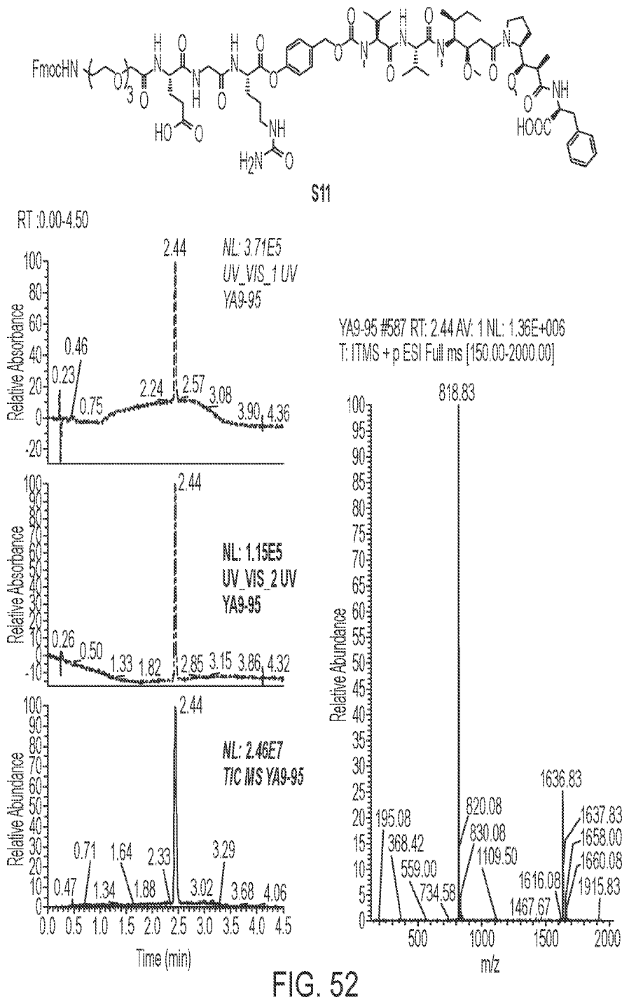

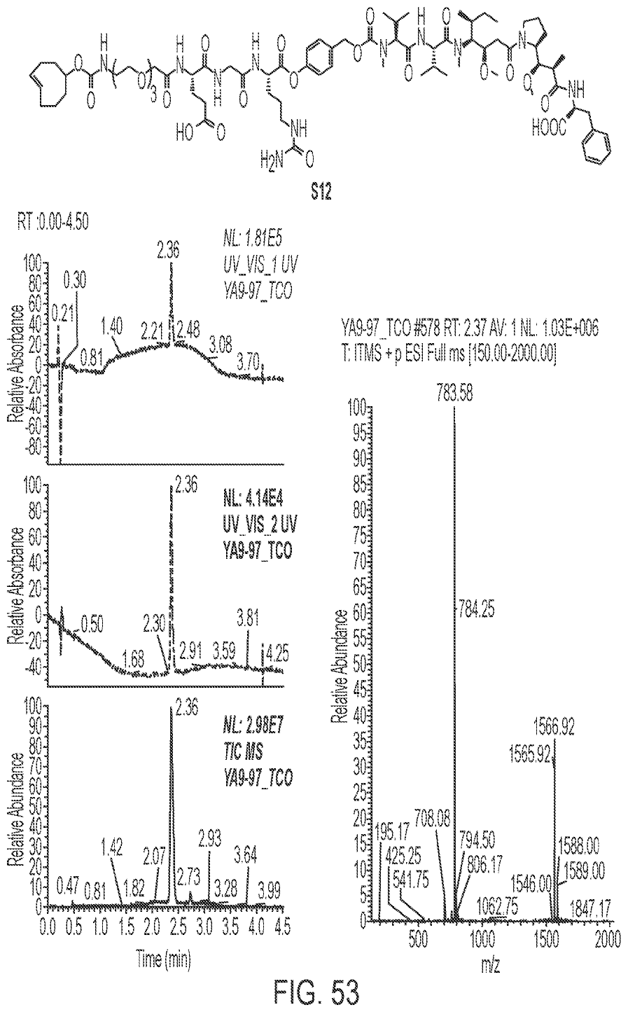

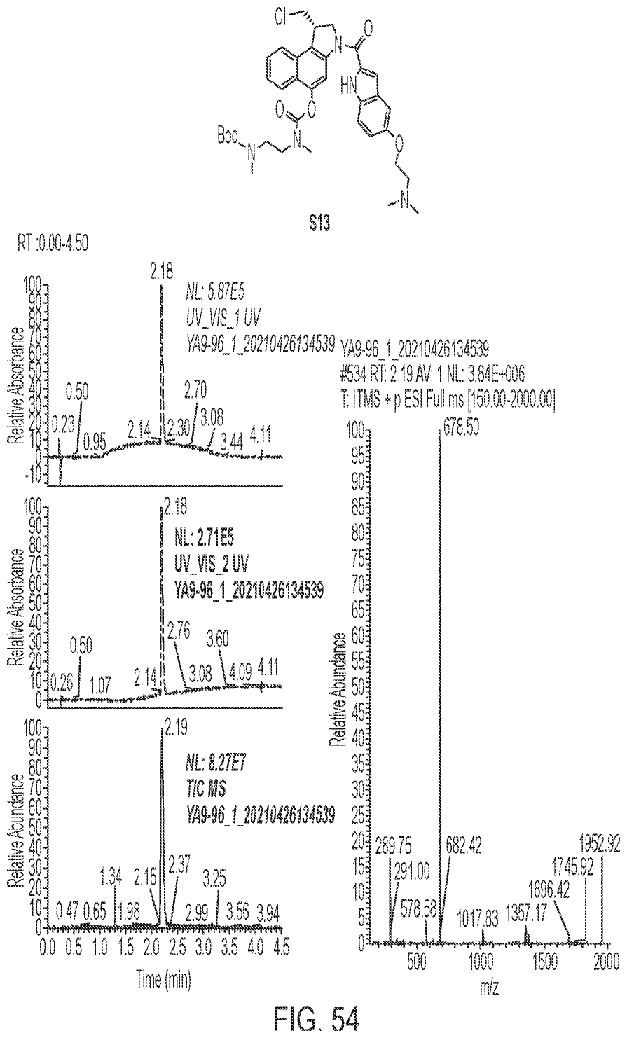

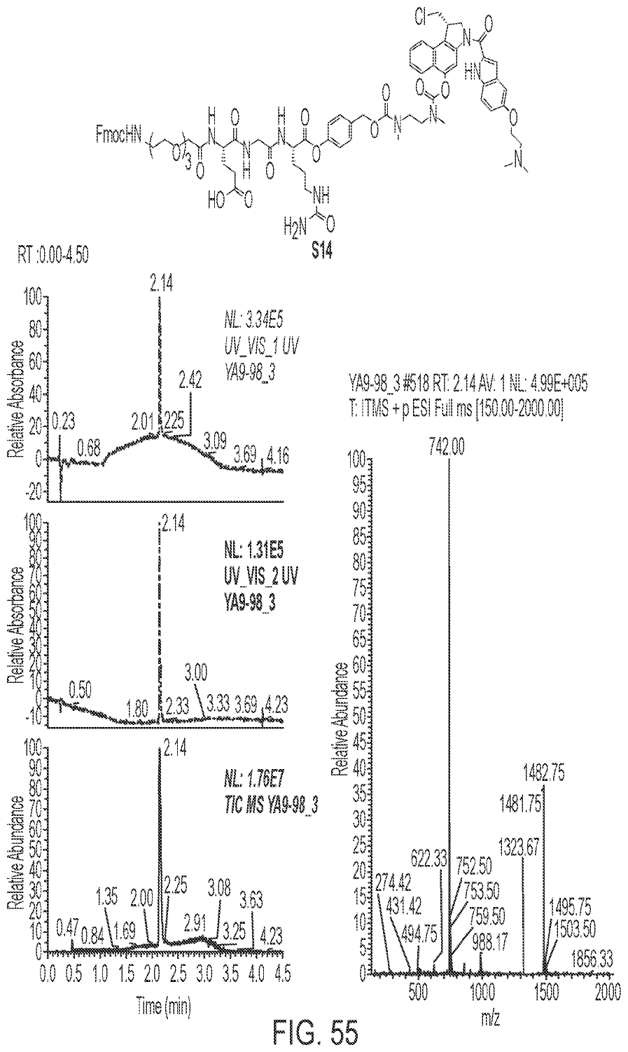

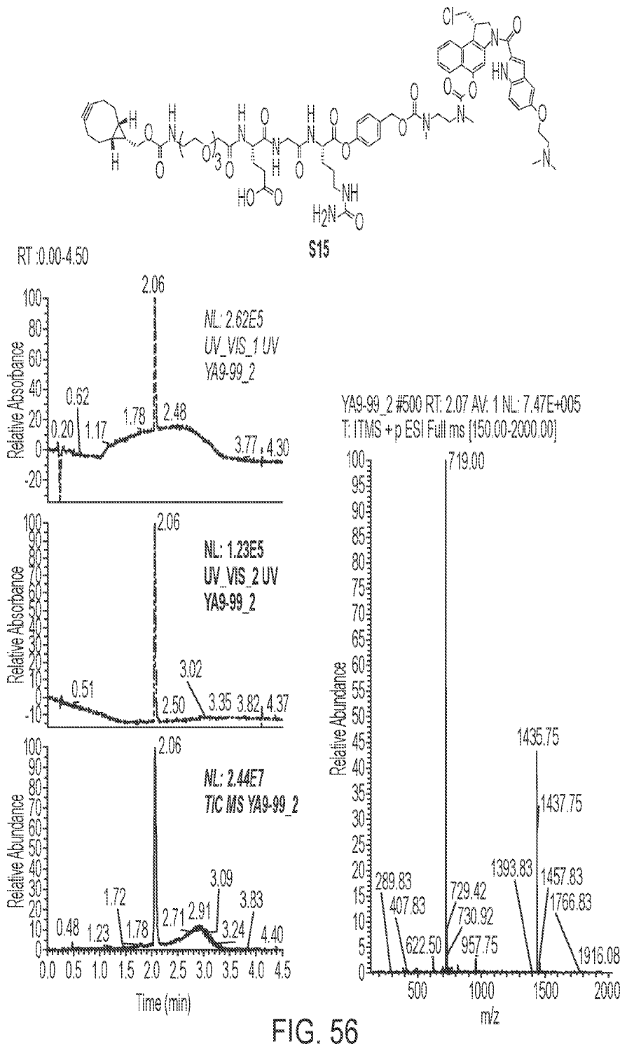

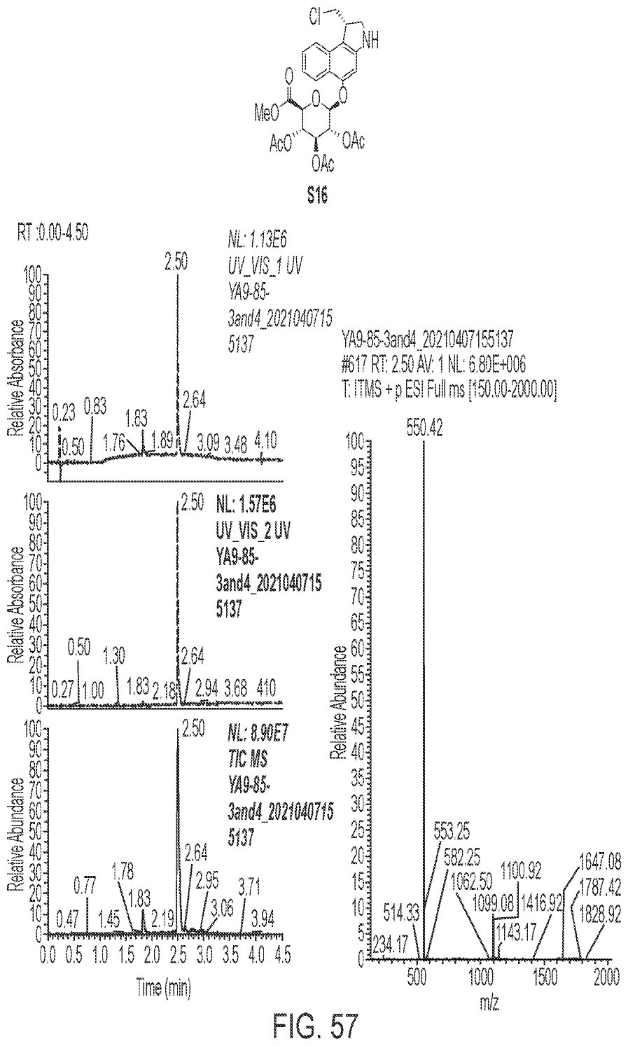

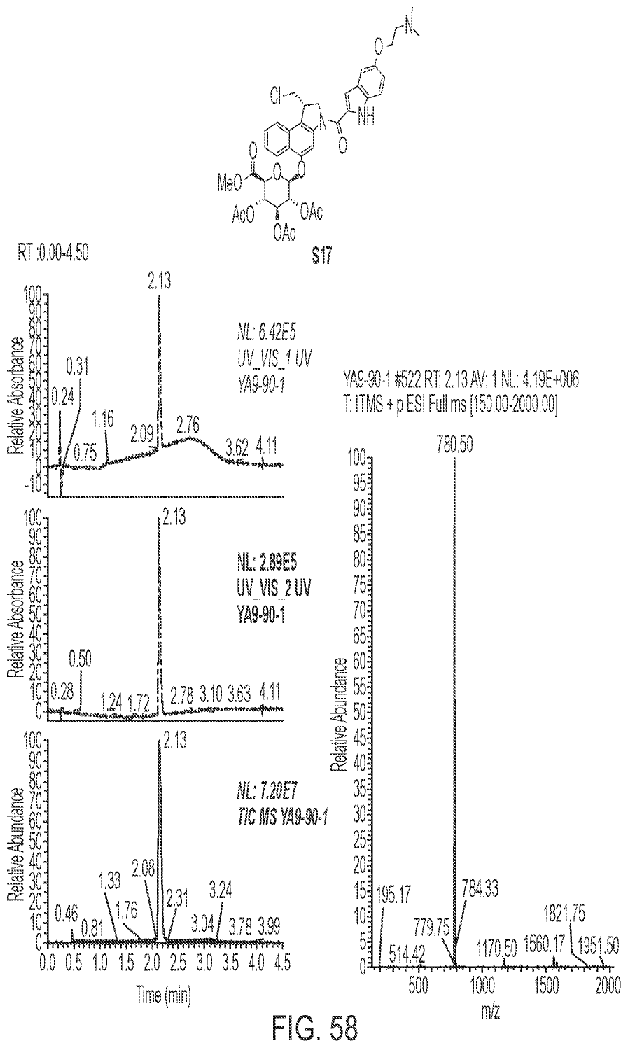

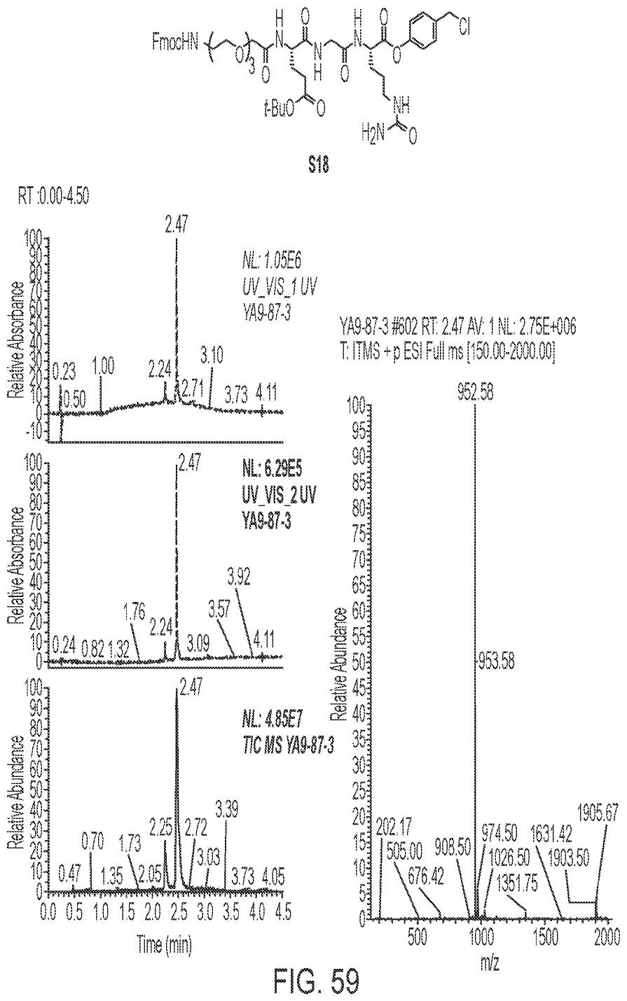

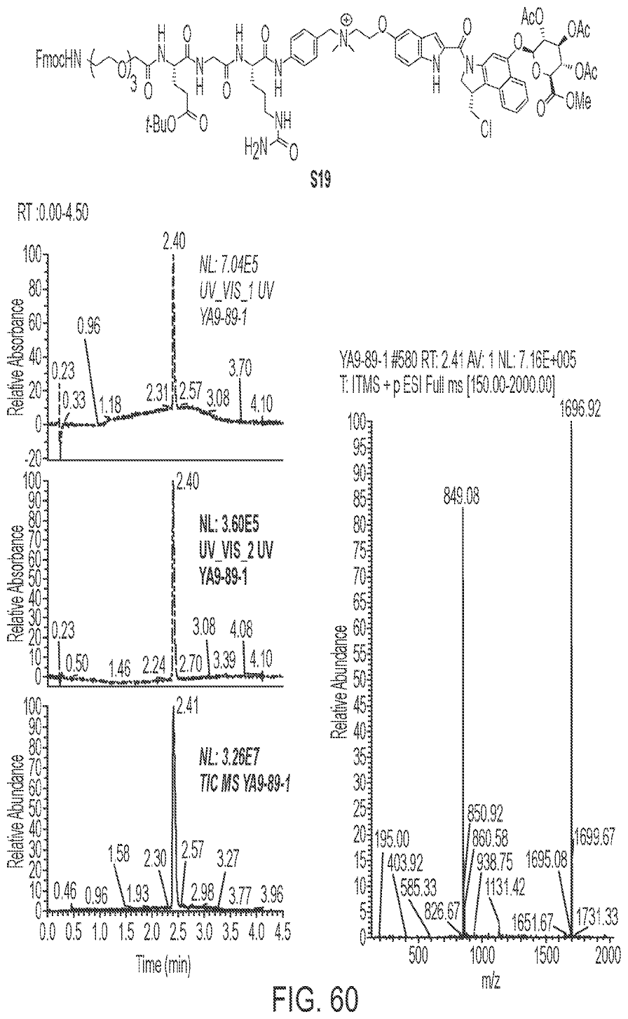

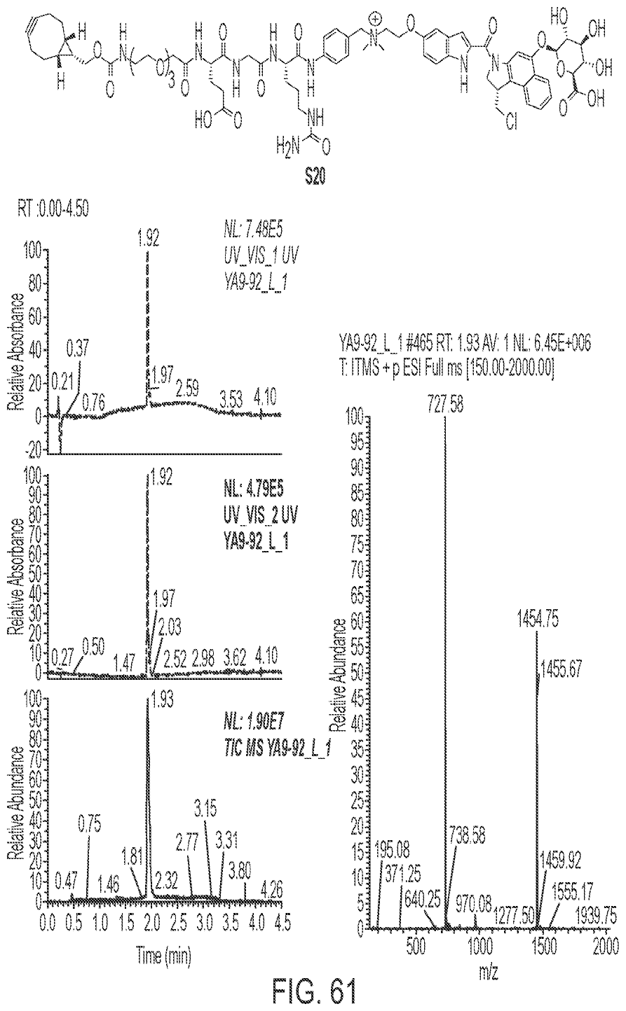

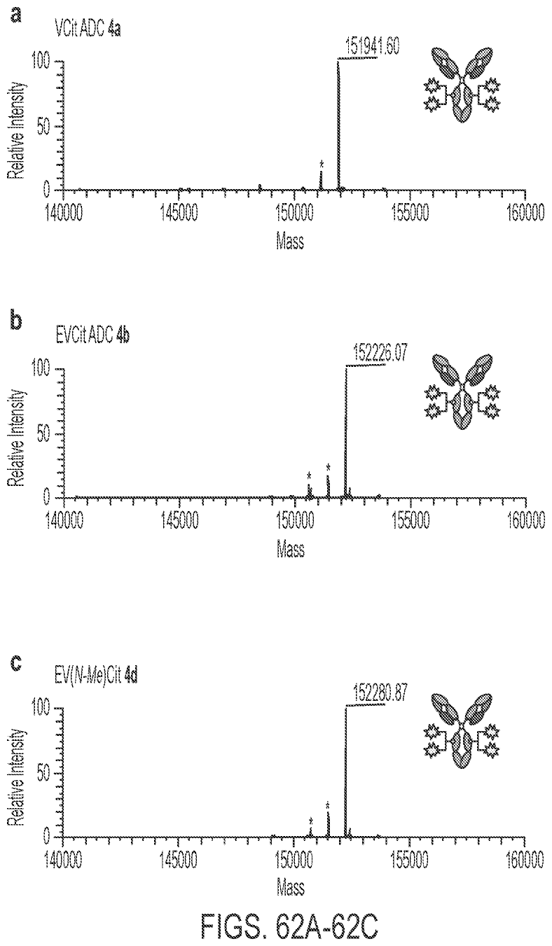

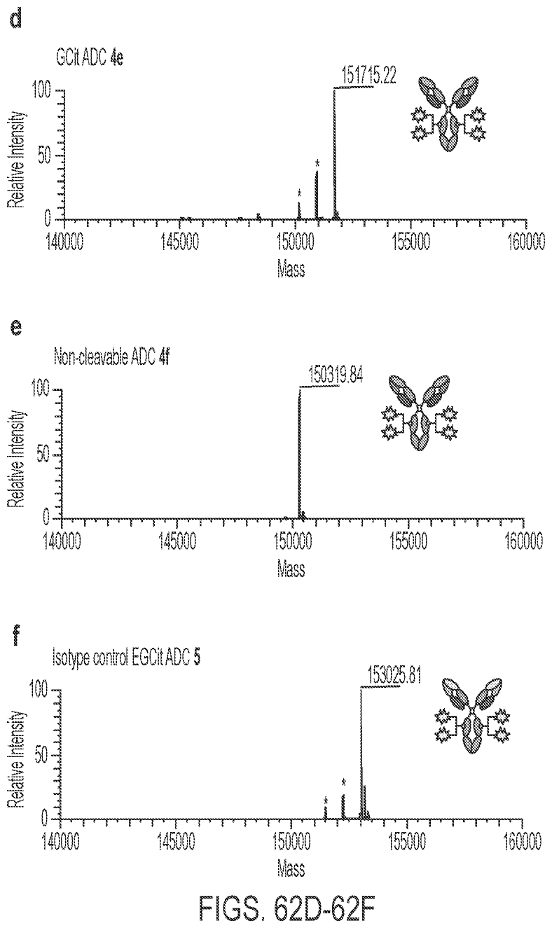

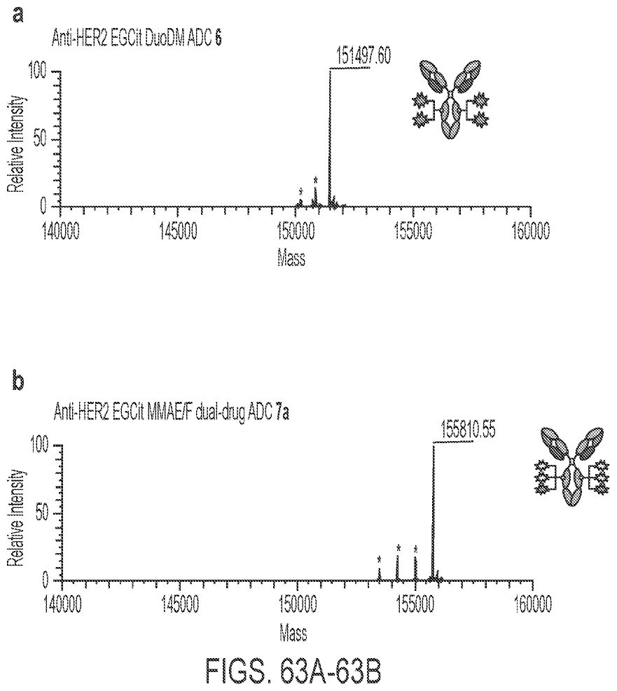

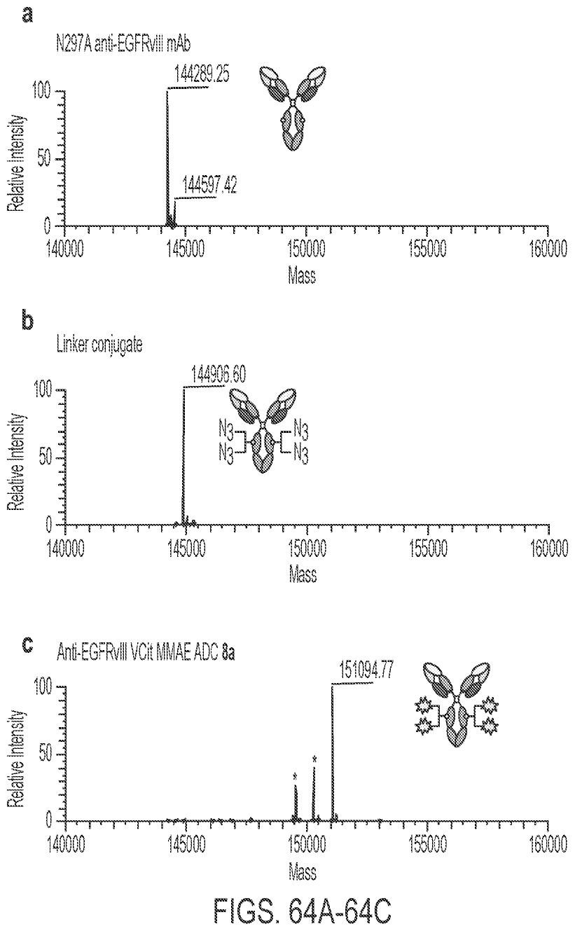

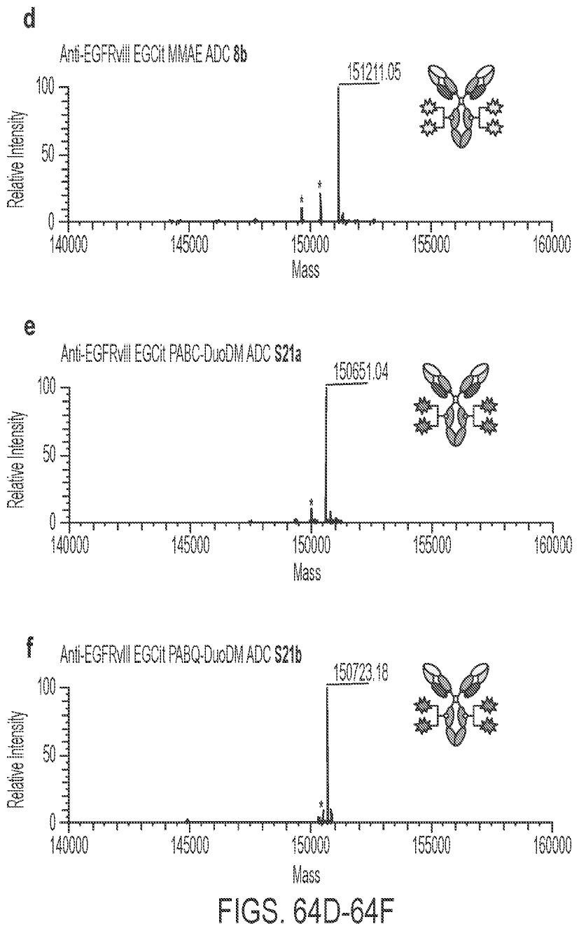

BCN-peg3-Glu-Gly-Cit-PAB-DuoDM-β-glucuronide (S20). (FIG. 19) Compound S19 (2.7 mg, 1.6 μmol) was dissolved in 20% TFA/DCM (200 μL and 800 μL). After being stirred at room temperature for 1 h, the solution was concentrated in vacuo and the crude peptide was precipitated with cold diethyl ether (20 mL) followed by centrifugation at 2,000×g for 3 min (3 times). The crude peptide was dissolved in MeOH (300 μL) and LiOH·H₂O (2 mg, 48 μmol) in water (300 μL) was added to the mixture. After being stirred at room temperature for 1 h, the mixture was cooled to 0° C. and quenched with formic acid (3 μL). Then, the solution was removed in vacuo and the crude peptide was dissolved in DMF (400 μL). BCN·NHS (0.6 mg, 2.1 μmol) and DIPEA (0.5 μL, 3.2 μmol) were added to the solution and the mixture was stirred at room temperature for 1.5 h. The crude products were purified by preparative RP-HPLC under basic conditions to afford analytically pure peptide S20 (1.0 mg, 42% for the 3 steps). Purity was confirmed by LC-MS. White powder. HRMS (ESI) Calcd. For C₇₁H₉₀N₁₀O₂₁Cl [M]⁺: 1453.5965, Found: 1453.5950.

Expression and purification of human monoclonal antibodies. Free style HEK-293 human embryonic kidney cells (Invitrogen) were transfected with a mammalian expression vector encoding for the human IgG1 kappa light chain and full length heavy chain sequences (based on the variable sequences of trastuzumab and depatuxizumab). A mutation of N297A was incorporated into the heavy chain constant region to produce aglycosylated mAbs. The transfected HEK-293 cells were cultured in a humidified cell culture incubator at 37° C. with 8% CO₂ and shaking at 150 rpm for 7 days before harvesting the culture medium. The antibody secreted into the culture medium was purified using Protein A resin (GE Healthcare).

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference:
U.S. Pat. No. 5,200,534
U.S. Pat. No. 5,202,448
U.S. Pat. No. 5,229,529
U.S. Pat. No. 5,274,137
U.S. Pat. No. 5,279,949
U.S. Pat. No. 5,283,253
U.S. Pat. No. 5,294,637
U.S. Pat. No. 5,415,869
U.S. Pat. No. 5,821,263
U.S. Pat. No. 5,824,701
U.S. Pat. No. 5,869,680
U.S. Pat. No. 6,214,345
U.S. Pat. No. 6,232,287
U.S. Pat. No. 6,323,315
U.S. Pat. No. 6,528,481
U.S. Pat. No. 6,570,040
U.S. Pat. No. 7,375,078
U.S. Pat. No. 7,452,964
U.S. Pat. No. 7,671,010
U.S. Pat. No. 7,691,962
U.S. Pat. No. 7,781,565
U.S. Pat. No. 8,450,278
U.S. Pat. No. 8,507,445
U.S. Pub. No. 2003/0096743
U.S. Pub. No. 2004/0005647
U.S. Pub. No. 2006/0034925
U.S. Pub. No. 2006/0115537
U.S. Pub. No. 2006/0223114
U.S. Pub. No. 2006/0234299
U.S. Pub. No. 2007/0148095
U.S. Pub. No. 2008/0279868
U.S. Pub. No. 2011/0053435
U.S. Pub. No. 2012/0141550
U.S. Pub. No. 2013/0138032
U.S. Pub. No. 2014/0024610
U.S. Pub. No. 2014/0087413
EP 1,391,213
EP 590,267
WO 2018/218004
WO 2008/083312
WO 2008/121949
WO 81/01145
WO 93/10076
WO 93/23555
WO 94/07876
WO 94/07880
WO 94/07881
WO 94/07882
WO 96/14856
WO 96/33212
WO 98/13059
WO 98/22451
WO 98/28288
WO 98/58927
WO 99/09021
WO 99/14209
WO 99/18113
Anami et al., Org. Biomol. Chem. 15:5635-5642, 2017.
Anami et al., Nat. Commun., 9:1, 2018.
Anami et al., Nat. Commun., 9:2512, 2018.
Anami et al., Methods Mol. Biol., 2078:71, 2020.
Anami et al., Mol. Cancer Ther., 19:2330, 2020.
Barclay et al. (eds.), The Leucocyte Antigen Facts Book, Academic Press, 1993.
Burke et al., Mol. Cancer Ther. 16(1):116-123, 2017.
Burkly et al., 2007.
Campbell et al., 1991.
Carl et al., J. Med. Chem., 24(3):479-480, 1981.
Deghenghi et al., Endocrine 14:29, 2001.
Doronina et al., Nature Biotechnology 21(7):778-784, 2003 (erratum, p. 941).
Dubowchik et al., Pharmacology & Therapeutics, 83:67-123, 1999.
Eggink et al., J. Biol. Chem., 284:26941, 2009.
Fu et al., Frontiers in Immunology, 9, 2018.
Greene and Wuts, Protective Groups in Organic Chemistry, 3rd Ed., 1999.
Hamblett et al., Mol. Cancer Ther., 14:1614, 2015.
Handbook of Pharmaceutical Salts: Properties, and Use (P. H. Stahl & C. G. Wermuth eds., Verlag Helvetica Chimica Acta, 2002).
Kurebayashi et al., Br. J. Cancer 79:707-717, 1999.
Liu et al. Proc. Natl. Acad. Sci., 93:8618-8623, 1996.
Lyon et al., Nat. Biotechnol., 33:733-735, 2015.
March's Advanced Organic Chemistry: Reactions, Mechanisms, and Structure, 2007.
Matos et al., 2010.
Nechushtan et al., 1997.
Onda et al., 2004.
Phillips et al., Mol. Cancer Ther., 15:661, 2016.
Remington's Pharmaceutical Sciences,” 15th Edition, pages 1035-1038 and 1570-1580.
Sjogren, J. Gastroenterol. Hepatol., 19:69, 2004.
Smith and March, March's Advanced Organic Chemistry: Reactions. Mechanisms, and Structure, Fifth Edition, Wiley-Interscience, 2001.
Staben et al. Nat. Chem. 8, 1112-1119, 2016.
Sun et al., Bioconjugate Chem. 28:1371-1381, 2017.
Thompson (ed.), The Cytokine Handbook, Academic Press, San Diego, 1994.
Told et al., J. Org. Chem. 67:1866-1872, 2002.
Vogel, A Textbook of Practical Organic Chemistry, Including Qualitative Organic Analysis Fourth Edition, New York: Longman, 1978.
Weitman et al., 1992.
Winkles 2008.
Winthrop et al., 2003.
Yamaguchi et al., Bioorg. Med. Chem., 32:116013, 2021.
Yamazaki et al., Nat. Commun., 12:3528, 2021.
Zhao et al., Mol. Cancer Ther., 16:1833, 2017.
Zhou et al., 2011.

1. A compound of Formula (I): 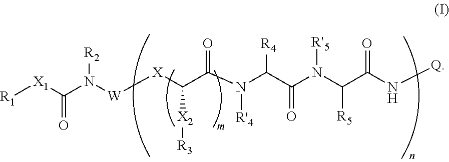
or a pharmaceutically acceptable salt thereof, wherein:
X₁ is a covalent bond, alkanediyl_{(C≤12)}, or substituted alkanediyl_{(C≤12)};
R₁ is hydrogen, —ZR₆, —(OCH₂CH₂)₀₋₅₀ZR₆, or substituted —(OCH₂CH₂)₀₋₅₀ZR₆, wherein:
R₆ is hydrogen, hydroxy, aminohydroxy, amino, mercapto, hydroxylamino, hydrazino, or azide;
alkyl_{(C≤12)}, alkenyl_{(C≤12)}, alkynyl_{(C≤12)}, alkylhydrazine_{(C≤12)}, or a substituted version thereof;
a polyglycine comprising from 1 to 6 glycine units; or
a substructure of the formula: 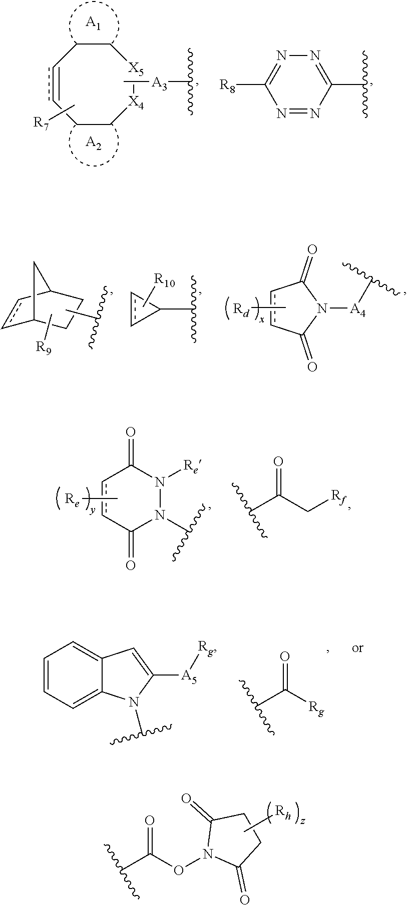
wherein:
A₁ and A₂ are each independently absent or arenediyl_{(C≤12)}, substituted arenediyl_{(C≤12)}, heteroarenediyl_{(C≤12)}, or substituted heteroarenediyl_{(C≤12)}, and form a fused arene_{(C≤12)}, substituted arene_{(C≤12)}, heteroarene_{(C≤12)}, or substituted heteroarene_{(C≤12)};
A₃ is a covalent bond, O, alkanediyl_{(C≤8)}, substituted alkanediyl_{(C≤8)}, alkoxydiyl_{(C≤8)}, or substituted alkoxydiyl_{(C≤8)};
A₄ and A₅ are each independently selected from a covalent bond, alkanediyl_{(C≤8)}, substituted alkanediyl_{(C≤8)}, arenediyl_{(C≤8)}, and substituted arenediyl_{(C≤8)};
R_{d}, Rₑ, Rₑ′, and Rₕ are each independently selected from hydrogen, halo, thioether, selenoether, sulfate, tosylate, mesylate, aryl_{(C≤8)}, and substituted aryl_{(C≤8)};
R_{f} is halo;
R_{g} is amine, hydrazine, alkylamino(ccs), substituted alkylamino(ccs), dialkylamino(ccs), substituted dialkylamino(ccs), alkylhydrazine_{(C≤8)}, or substituted alkylhydrazine_{(C≤8)};
X₄ and X₅ are each independently O, N, C(O), or CH₂, or X₄ and X₅ are alkanediyl_{(C≤8)} or substituted alkanediyl_{(C≤8)} and are taken together to form a fused cycloalkane group consisting of 3 to 8 ring atoms;
R₇ is hydrogen, hydroxy, amino, or oxo;
R₈ is carboxy; or
alkyl_{(C≤12)}, amido_{(C≤12)}, aryl_{(C≤12)}, heteroaryl_{(C≤12)}, —C(O)OR₁₁, —C(O)NR₁₁R₁₁′, or a substituted version thereof, wherein:
R₁₁ and R₁₁′ are each independently hydrogen; or
alkyl_{(C≤12)}, aryl_{(C≤12)}, or a substituted version thereof;
R₉ and R₁₀ are each independently hydroxy, amino, or halo; or
alkyl_{(C≤12)}, aryl_{(C≤12)}, or a substituted version thereof;
x is 0-4, as valency permits;
y is 0-4, as valency permits;
z is 0-4;
Z is a covalent bond, alkanediyl_{(C≤12)}, —C(O)-alkanediyl_{(C≤12)}, —C(O)-alkanediyl_{(C≤12)}—C(O)NH—, or a substituted version thereof;
R₂ is hydrogen, alkyl_{(C≤12)}, substituted alkyl_{(C≤12)}, aryl_{(C≤12)}, heteroaryl_{(C≤12)}, aralkyl_{(C≤12)}, heteroaralkyl_{(C≤12)}, acyl_{(C≤12)}, substituted acyl_{(C≤12)}, or a monovalent amino protecting group;
W is a covalent bond or a polyvalent polymer having 2-21 connection points;
n is 1 to 20 provided that: when W is a covalent bond, then n is 1; and when W is a polyvalent polymer, then n is less than or equal to one less than the number of connection points;
each X is independently a covalent bond, alkanediyl_{(C≤12)}, substituted alkanediyl_{(C≤12)}, one or more amino acid residues, or an oligomeric peptide;
each X₂ is independently alkanediyl_{(C≤12)} or substituted alkanediyl_{(C≤12)};
each R₃ is independently hydroxy or amino;
alkoxy_{(C≤12)}, acyloxy_{(C≤12)}, alkylamino_{(C≤12)}, dialkylamino_{(C≤12)}, amido_{(C≤12)}, heteroaryl_{(C≤12)}, or a substituted version thereof; or
—X₆—C(O)R₁₂, wherein:
X₆ is O, —NR_{b}—, or a covalent bond;
R_{b} is hydrogen, alkyl_{(C≤6)}, substituted alkyl_{(C≤6)}, or a monovalent amino protecting group;
R₁₂ is hydroxy or amino; or
alkoxy_{(C≤12)}, alkylamino_{(C≤12)}, dialkylamino_{(C≤12)}, or a substituted version thereof; or
—OSO₂NR₁₃R₁₃′, —OP(O)(OH)OR₁₄, —OSO₂OR₁₄′, —NR_{c}—SO₂NR₁₃R₁₃′, —NR_{c}—P(O)(OH)OR₁₄, —NR_{c}—SO₂OR₁₄′, —SO₂NR₁₃R₁₃′, —P(O)(OH)OR₁₄, or —SO₂OR₁₄′, wherein:
R_{c} is hydrogen, alkyl_{(C≤6)}, substituted alkyl_{(C≤6)}, aryl_{(C≤12)} heteroaryl_{(C≤12)}, aralkyl_{(C≤12)}, heteroaralkyl_{(C≤12)}, or a monovalent amino protecting group;
R₁₃, R₁₃′, R₁₄, and R₁₄′ are each independently hydrogen, alkyl_{(C≤12)}, cycloalkyl_{(C≤12)}, aryl_{(C≤12)}, heteroaryl_{(C≤12)}, aralkyl_{(C≤12)}, heteroaralkyl_{(C≤12)}, or a substituted version thereof;
each m is independently 0 or 1;
each R₄ is independently the side chain moiety of glycine or valine; and
each R₄′ is hydrogen, alkyl_{(C≤12)}, substituted alkyl_{(C≤12)}, aryl_{(C≤12)}, heteroaryl_{(C≤12)}, aralkyl_{(C≤12)}, heteroaralkyl_{(C≤12)}, acyl_{(C≤12)}, substituted acyl_{(C≤12)}, or a monovalent amino protecting group;
each R₅ is independently the side chain moiety of glycine, alanine, ornithine, lysine, arginine, citrulline, asparagine, or glutamine, or an amino-protected version thereof;
each R₅′ is hydrogen, alkyl_{(C≤12)}, substituted alkyl_{(C≤12)}, aryl_{(C≤12)}, heteroaryl_{(C≤12)}, aralkyl_{(C≤12)}, heteroaralkyl_{(C≤12)}, acyl_{(C≤12)}, substituted acyl_{(C≤12)}, or a monovalent amino protecting group;
Q is a group of the formula: 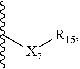
wherein:
R₁₅ is hydrogen, —R₁₆, or —C(O)—R₁₆, wherein:
R₁₆ is a therapeutic agent or an imaging agent;
X₇ is a covalent bond, O, S, —NH—, alkanediyl_{(C≤12)}, substituted alkanediyl_{(C≤12)}, —(OCH₂CH₂)ₚ—, or substituted —(OCH₂CH₂)ₚ—, wherein:
p is 0-50; or
a group of the formula: 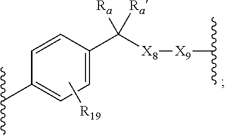
wherein:
Rₐ and Rₐ′ are each independently hydrogen, alkyl_{(C≤12)}, substituted alkyl_{(C≤12)}, aryl_{(C≤12)}, or heteroaryl_{(C≤12)};
X₈ is O or —NR₁₇R₁₇′—, wherein:
R₁₇ and R₁₇′ are each independently alkyl_{(C≤12)} or substituted alkyl_{(C≤12)};
R₁₉ is hydrogen, sugar, or a sugar derivative; and
X₉ is a covalent bond, O, S, —NH—, —(OCH₂CH₂)_{q}, or substituted —(OCH₂CH₂)_{q}, wherein:
q is 0-50; or
a group of the formula:
Y₁ 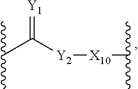
wherein:
Y₁ is O or S;
Y₂ is a covalent bond, O, S, —NH—, or —NR₁₈—, wherein:
R₁₈ is alkyl_{(C≤12)} or substituted alkyl_{(C≤12)}; and
X₁₀ is a covalent bond, alkyl_{(C≤12)}, substituted alkyl_{(C≤12)}, O, S, —NH—, —(CH₂CH₂O)ᵣ—, substituted —(CH₂CH₂O)ᵣ—, —(CH₂CH₂NR₂₀)ᵣ—, or substituted —(CH₂CH₂NR₂₀)ᵣ—;
r is 0-50;
R₂₀ is hydrogen, alkyl_{(C≤12)}, or substituted alkyl_{(C≤12)};
provided that when R₄ is valine, then R₄′ is not hydrogen; or
a compound selected from: 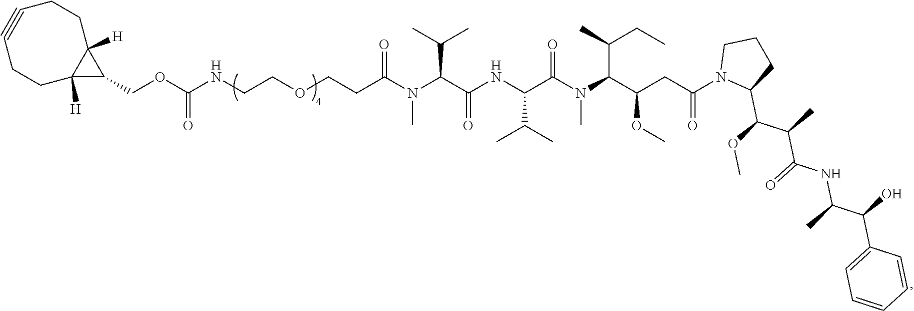
and
and pharmaceutically acceptable salts thereof.

2.-10. (canceled)

11. The compound of claim 1, wherein W is a polyvalent polymer with 2-5 connection points.

12.-13. (canceled)

14. The compound of claim 1, wherein X₂ is alkanediyl_{(C≤12)}.

15. (canceled)

16. The compound of claim 1, wherein R₃ is —X₆—C(O)R₁₂, wherein: X₆ is O, —NR_{b}—, or a covalent bond; R_{b} is hydrogen, alkyl_{(C≤6)}, substituted alkyl_{(C≤6)}, or a monovalent amino protecting group; and R₁₂ is hydroxy or amino, or alkoxy_{(C≤12)}, alkylamino_{(C≤12)}, dialkylamino_{(C≤12)}, or a substituted version thereof.

17.-20. (canceled)

21. The compound of claim 1, wherein each R₄ is the side chain of glycine.

22. (canceled)

23. The compound of claim 1, wherein each R₅ is the side chain of citrulline.

24.-42. (canceled)

43. A drug conjugate comprising:
(A) a drug moiety, wherein prior to attachment, the drug moiety is the compound of claim 1;
(B) a linker; and
(C) a cell targeting group.

44. The drug conjugate of claim 43, wherein the cell targeting group is an antibody, antibody fragment, protein, or small molecule.

45.-48. (canceled)

49. The drug conjugate of claim 43, wherein the linker is:
(A) a non-covalent bond formed by hydrogen bonding, nucleobase pairing, electrostatic interactions, pi stacking, van der Waals interactions, or dipole-dipole interactions;
(B) a covalent bond;
(C) a monovalent spacer comprising 1 connection point; or
(D) a polyvalent spacer comprising 2-21 connection points.

50.-54. (canceled)

55. The drug conjugate of claim 43, wherein prior to attachment, the linker is a compound of Formula (V): 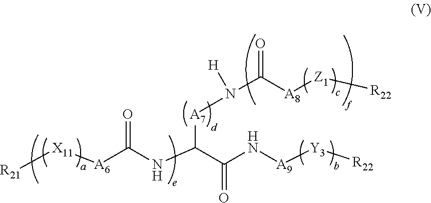
or a pharmaceutically acceptable salt thereof, wherein:
A₆, A₇, A₈, and A₉ are each independently alkanediyl_{C1-12}, arenediyl_{C1-12}, heteroarenediyl_{C1-12}, cycloalkanediyl_{C1-12}, heterocycloalkanediyl_{C1-12}, or a substituted version thereof, or a side chain group of a canonical amino acid;
X₁₁, Y₃, and Z₁ are each independently a covalent bond, —[O(CH₂)_{q}]—, —[O(CHW₁′)_{q}]—, or —[O(CW₁′W₁″)_{q}]—;
wherein:
W₁′ and W₁″ are each independently amino, hydroxy, halo, mercapto, alkyl_{C1-12}, cycloalkyl_{C1-12}, alkenyl_{C1-12}, alkynyl_{C1-12}, aryl_{C1-12}, aralkyl_{C1-12}, heteroaryl_{C1-12}, heteroaralkyl_{C1-12}, heterocycloalkyl_{C1-12}, acyl_{C1-12}, acyloxy_{C1-12}, or alkylamino_{C1-12}, or a substituted version thereof;
q is 1-3;
a, b, c, and d are each independently 0-12;
e and f are each independently 0, 1, 2, or 3;
R₂₁, R₂₂, and R₂₃ are each independently hydrogen, —NH₂, —NHR₂₄, —NR₂₄R₂₅, —N₃, heteroaryl_{(C≤12)}, substituted heteroaryl_{(C≤12)}, -arenediyl_{(C≤12)}-heteroaryl_{(C≤12)}, substituted -arenediyl_{(C≤12)}-heteroaryl_{(C≤12)}, or a conjugating group;
wherein:
R₂₄ and R₂₅ are each independently alkyl_{C1-12}, cycloalkyl_{C1-12}, alkenyl_{C1-12}, alkynyl_{C1-12}, aryl_{C1-12}, aralkyl_{C1-12}, heteroaryl_{C1-12}, heteroaralkyl_{C1-12}, heterocycloalkyl_{C1-12}, acyl_{C1-12}, acyloxy_{C1-12}, alkylamino_{C1-12}, or a substituted version thereof, or a monovalent amino protecting group; or
R₂₄ and R₂₅ are taken together and is a divalent amino protecting group; and
provided that: at least one of R₂₁, R₂₂, and R₂₃ is —NH₂ or a group containing —NH₂; and at least one of R₂₁, R₂₂, and R₂₃ is —N₃, heteroaryl_{(C≤12)}, or -arenediyl_{(C≤12)}-heteroaryl_{(C≤12)}.

56. The drug conjugate of claim 55, wherein A₆, A₇, A₈, and A₉ are each independently alkanediyl_{C1-12} or substituted alkanediyl_{C1-12}.

57.-67. (canceled)

68. The drug conjugate of claim 55, wherein X₁₁, Y₃, and Z₁ are each independently —[O(CH₂)_{q}]—.

69.-86. (canceled)

87. The drug conjugate of claim 55, wherein R₂₁ is —NH₂ or —N₃; R₂₂ is —N₃; and R₂₃ is hydrogen, —N₃, heteroaryl_{(C≤12)}, substituted heteroaryl_{(C≤12)}, -arenediyl_{(C≤12)}-heteroaryl_{(C≤12)}, or substituted -arenediyl_{(C≤12)}-heteroaryl_{(C≤12)}.

88.-97. (canceled)

98. The drug conjugate of claim 43, wherein prior to attachment, the linker is: 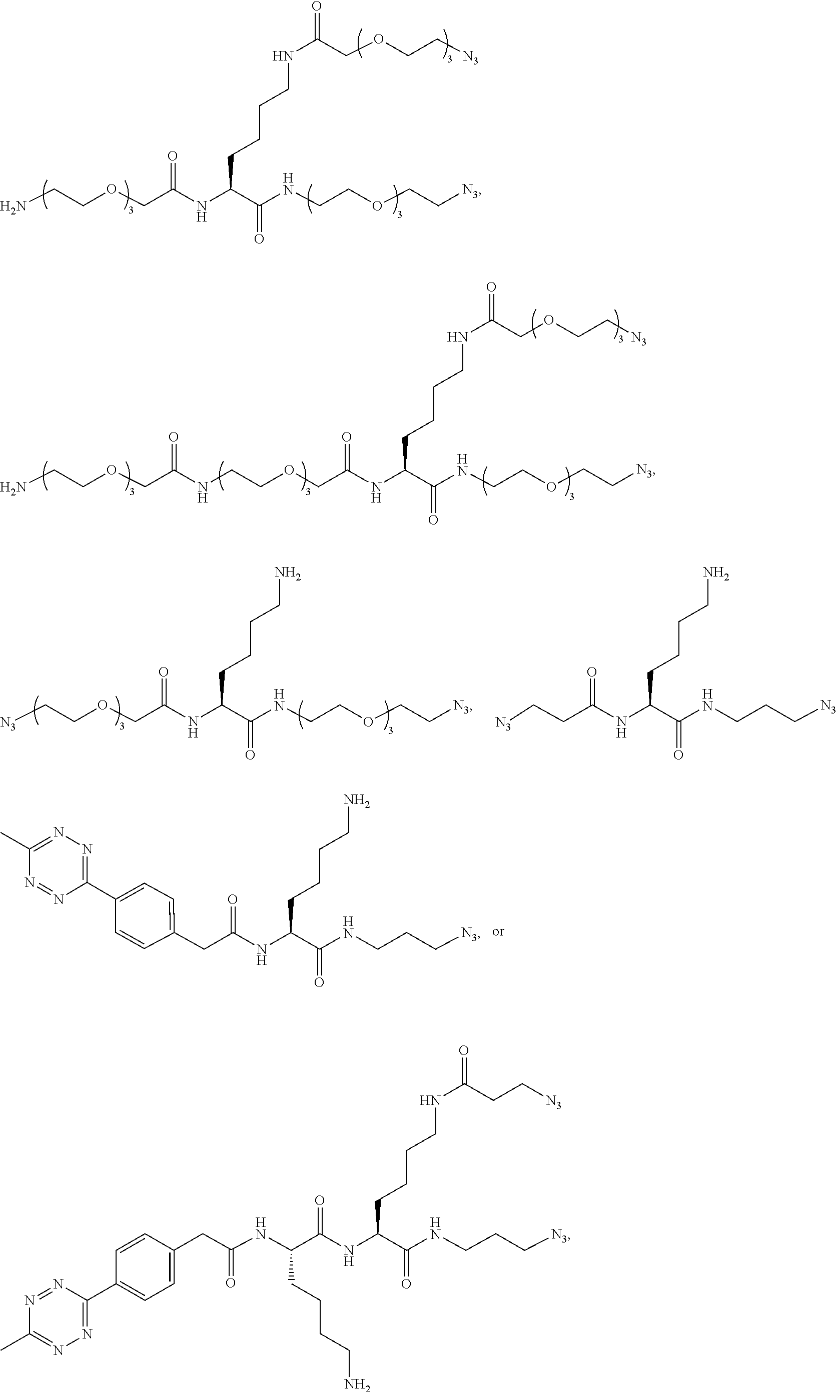
or a pharmaceutically acceptable salt thereof.

99. The drug conjugate of claim 43, wherein the compound comprises R₁₆ as a chemotherapeutic drug.

100. The drug conjugate of claim 99, wherein the chemotherapeutic drug is auristatin E, auristatin F, monomethyl auristatin E, monomethyl auristatin F, dolastatine, maytansine, duocarmycin, tubulysin, chalicheamicin, pyrrobenzodiazepine dimer, anthracycline, paclitaxel, vinblastine, amanitin, or a derivative thereof.

101.-105. (canceled)

106. A pharmaceutical composition comprising the drug conjugate of claim 43 and an excipient.

107. The pharmaceutical composition of claim 106, wherein the pharmaceutical composition is formulated for oral, intraadiposal, intraarterial, intraarticular, intracranial, intradermal, intralesional, intramuscular, intranasal, intraocular, intrapericarial, intraperitoneal, intrapleural, intraprostatical, intrarectal, intrathecal, intratracheal, intratumoral, intraumbilical, intravaginal, intravenous, intraventricular, intravesicularal, intravitreal, liposomal, local, mucosal, parenteral, rectal, subconjunctival, subcutaneous, sublingual, topical, transbuccal, transdermal, or vaginal administration, or administration via a catheter, via a lavage, via continuous infusion, via infusion, via inhalation, via injection, via local delivery, or via localized perfusion.

108. A method of treating a disease or disorder in a patient comprising administering the drug conjugate of claim 43.

109.-123. (canceled)

124. The compound of claim 1, wherein the compound is: 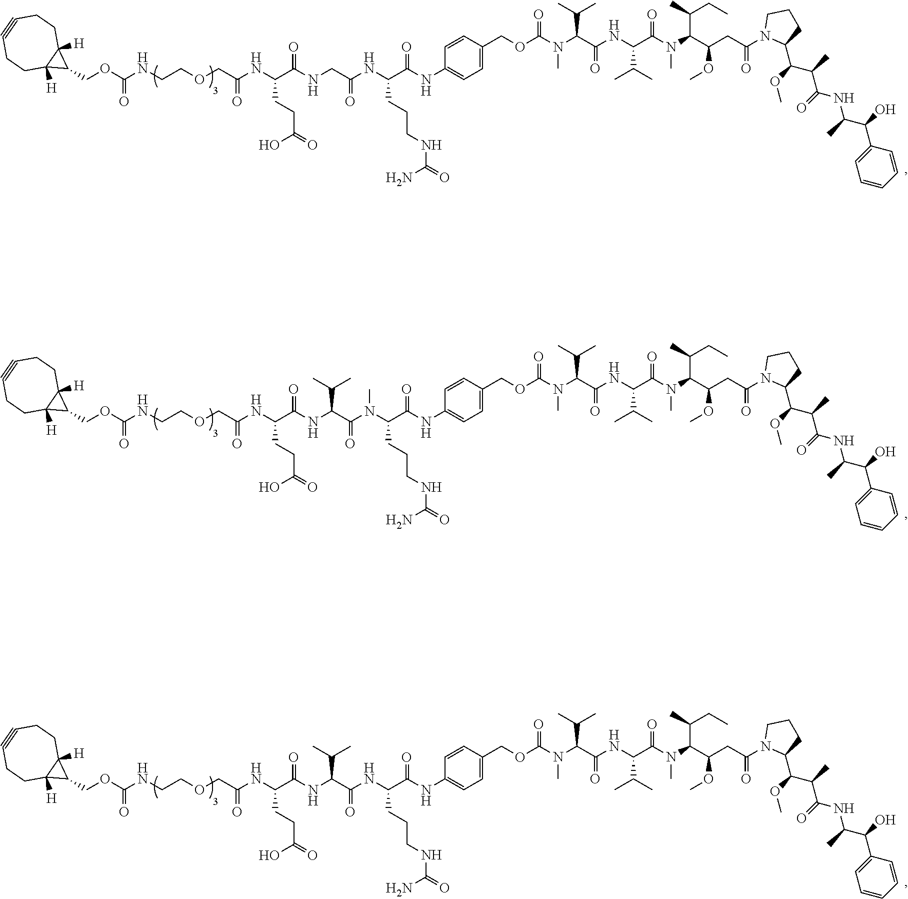 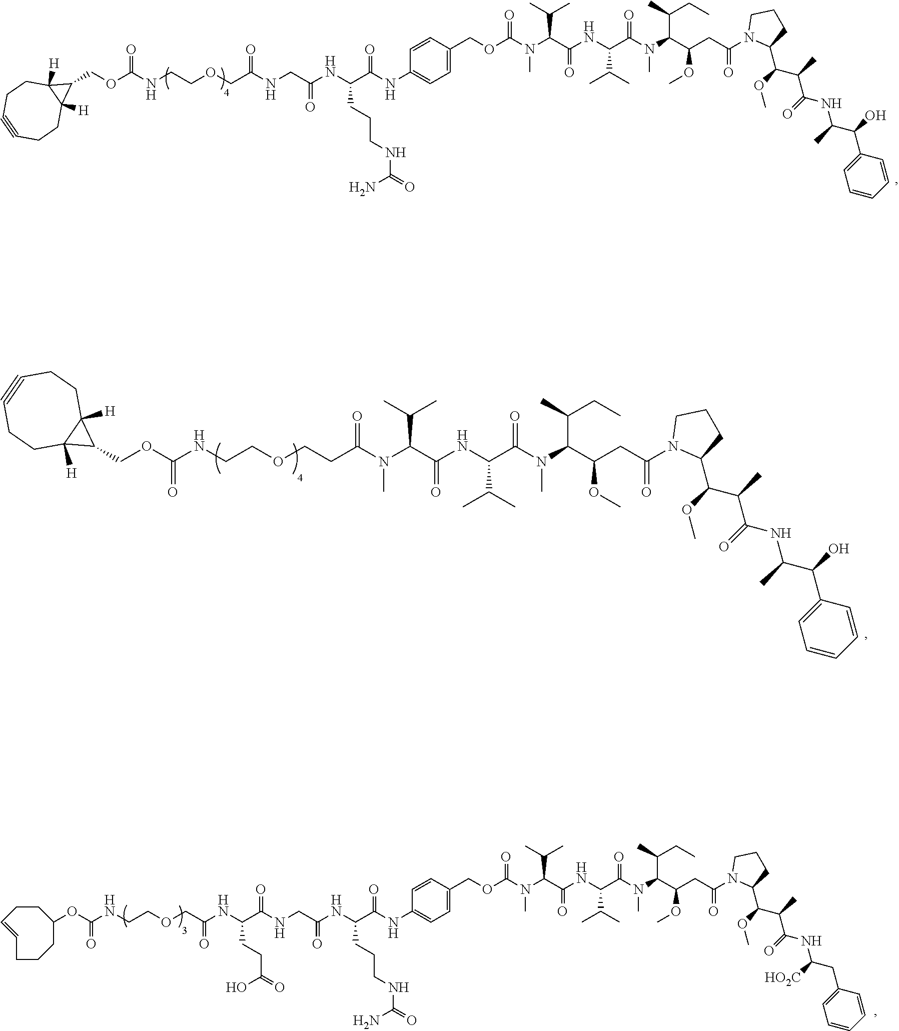 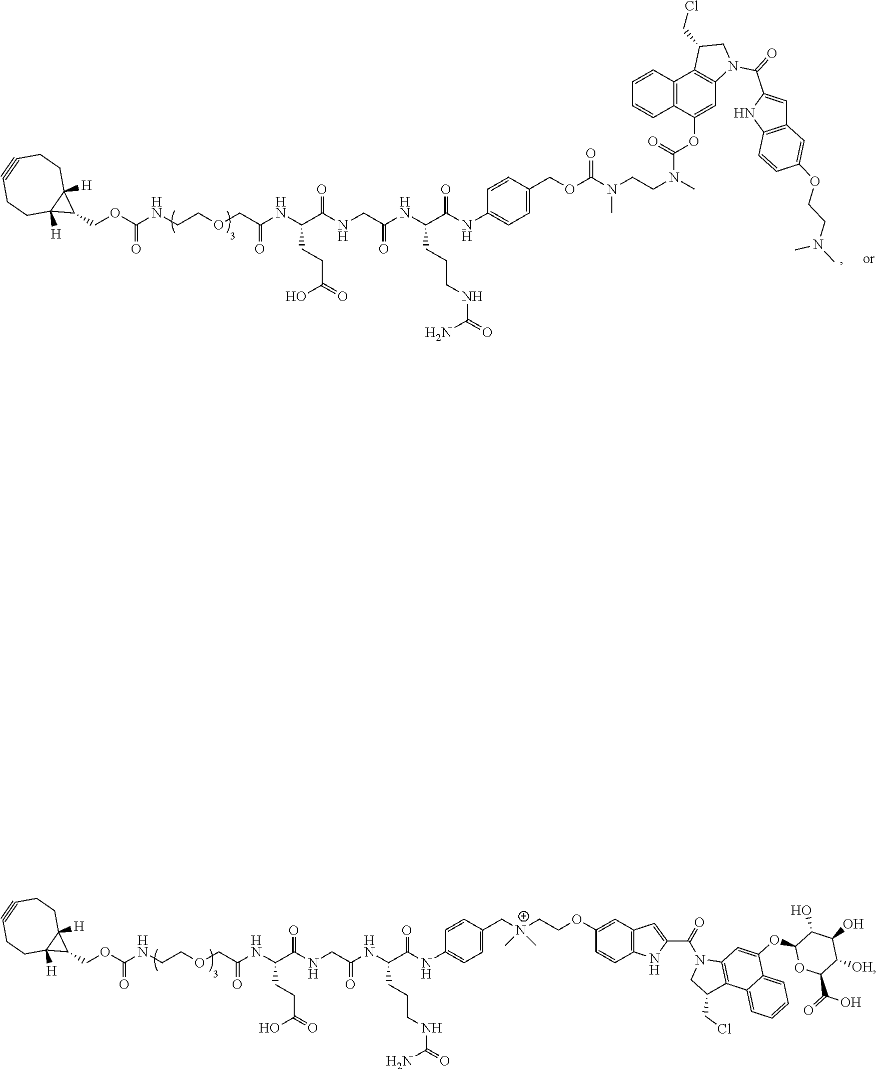
or a pharmaceutically acceptable salt hereof.
